(19) United States
(12) Patent Application Publication
(10) Pub. No.: US 2006/0073117 A1
(43) Pub. Date: Apr. 6, 2006

(54) Methods and compositions for controlling hair follicle stem cell fate

(75) Inventor: Linheng Li, Leawood, KS (US)

Correspondence Address: POLSINELLI SHALTON WELTE SUELTHAUS P.C., 700 W. 47TH STREET, SUITE 1000, KANSAS CITY, MO, 64112-1802, US

(73) Assignee: Stowers Institute for Medical Research

(21) Appl. No.: 10/956,882

(22) Filed: Oct. 1, 2004

(51) Int. Cl. A61K 39/395; A61K 35/14; C12N 5/08; C12N 5/06; C07H 21/04

(52) U.S. Cl. 424/93.1; 424/145.1; 435/372; 435/326; 530/388.25; 536/23.53

(57) ABSTRACT

The present invention relates to mutant hair follicle stem cells possessing an inactive Bmpr1a receptor, wherein such mutant hair follicle stem cells proliferate and undergo self-renewal. Additionally, the present invention relates to compositions and methods for induction of hair follicle stem cell proliferation in vivo and in vitro. The present invention also relates to mutant Bmpr1a organisms and tumor cells.

### FIELD OF INVENTION

The present invention relates to methods and compositions that may be employed to control hair follicle stem cell (HFSC) population expansion in vivo and in vitro. In particular, the present invention is directed to methods and compositions that may be utilized to control HFSC fate determination by modulating BMP signaling pathways.

### BACKGROUND OF INVENTION

Hair follicle stem cells (HFSCs) play a critical role in governing hair growth and maintaining the epidermis. HFSCs exist in the bulge area, which is situated in the middle of the hair follicle (HF). The bulge exists as a small swell of the hair follicle that forms the attachment site for the erector pili muscle. The wild type HFSC generates the entire structure of the hair, which includes a hair bulb (HB), dermal papilla (DP), dermal sheath (DS), precortex (PC), inner root sheath (IRS), outer root sheath (ORS), hair shaft (HS), bulge (Bu), arrector pili muscle (APM), and sebaceous gland (SG). The multipotent HFSC differentiates into various mature cells constituting the foregoing structures.

Adult hair follicles transition through cyclic phases of active proliferation, termed anagen phase, and periods of quiescence, termed telogen phase, with no proliferation occurring when the follicles contain a mature hair. In the mouse, these cycles of growth last 21 days, and therefore hair growth occurs at specific times in the life of the mouse. In adult humans, each hair follicle tends to behave autonomously; however, cycles of growth and dormancy still exist, occasionally lasting months. When the follicle is in growth phase, HFSC proliferative activity greatly increases at the base in the germinal or matrix region.

In the middle of anagen phase, bulge region-derived HFSCs migrate downward along the ORS to settle at the periphery of the HF bulb. Under the influence of the follicular papilla (FP), these cells transform into the hair germ and acquire the ability to respond to FP signaling to produce a new hair shaft. HFSCs have been characterized as functioning broadly in forming the hair follicle, sebaceous glands, and epidermis.

HFSCs possess the properties of self-renewal, proliferation and multilineage potentials and are a pluripotent, undifferentiated form of hair follicle cells. Considerable progress has been made in the past few decades in the identification of factors involved in supporting HFSC growth, proliferation, and differentiation. However, to date, the relative inability to expand the HFSC population in vivo and in vitro has greatly hindered mechanistic studies of stem cell properties and imposed limitations on the use of these cells in transplantation for generation of new hair growth. There is a lack of adequate in vivo information regarding the regulatory signal mechanisms involved in control of HFSC population expansion, self-renewal, proliferation, and differentiation.

The regulatory signals for modulation of HFSC growth, proliferation, and differentiation have been largely uncharacterized. At present, it is known that Bmpr1a receptors on stem cells, including HFSCs, bind bone morphogenic proteins (BMPs). The process by which this interaction affects HFSC growth is unknown. Thus, identification of HFSC Bmpr1a cell receptor interactions with regulatory proteins and polypeptides, such as bone morphogenic protein (BMP), Noggin, and polypeptide fragments thereof, will permit a better understanding of potential methods for controlling and promoting HFSC proliferation.

It is desired to have a viable conditional mutant Bmpr1a organism that possesses an inactive Bmpr1a cell surface receptor encoded by a mutant Bmpr1a gene for investigation of the impact of Bmpr1a upon HFSC growth, proliferation, and differentiation in vivo. As such, the inactive Bmpr1a receptor would be unable to bind to BMP or Noggin. Moreover, model Bmpr1a mutant organisms for in vivo and in vitro analyses of HFSCs are desired. Significantly, it is desired to develop compositions and methods for the induction of HFSC self-renewal, proliferation, growth, and differentiation within the hair follicle (HF) architectural structure.

Related to this, a useful molecular biology tool would be a viable Bmpr1a conditional knockout mouse, since null homozygous Bmpr1a allele-containing mutant mice are embryonically lethal, dying at day 8 without mesoderm formation. At present, lethality of the null Bmpr1a mutant mouse has hampered investigation of Bmpr1a cell receptors and their role in modulating HFSC expansion and differentiation in postnatal stages of hair development.

Molecular biology tools are desired for studying Bmpr1a receptors and their interactions with regulatory molecules. Desired tools include mutant Bmpr1a nucleic acid sequences, inactive Bmpr1a polypeptides, Bmpr1a antisense nucleic acid sequences, isolated Noggin polypeptides, vectors containing mutant Bmpr1a nucleic acid sequences, anti-Bmpr1a receptor antibodies, anti-BMP antibodies, and fragments thereof. In vitro cell cultivation systems are also desired for expansion of wild type HFSCs and mutant HFSCs containing inactive Bmpr1a receptor polypeptides. Methods for making and using the foregoing Bmpr1a genes, Bmpr1a polypeptides, vectors, Bmpr1a mutant organisms, HFSCs, tumors, and molecular biology tools are desired.

### SUMMARY OF INVENTION

The present invention relates to a mutant hair follicle stem cell (HFSC) containing an isolated mutant Bmpr1a nucleic acid sequence that encodes an inactive Bmpr1a receptor. The isolated mutant Bmpr1a nucleic acid sequence can contain a mutation such as a frame shift, substitution, loss of function, knockout deletion, or conventional deletion mutations. The present invention also relates to a mutant HFSC containing a truncated Bmpr1a nucleic acid sequence that is lacking Exon 2 of the Bmpr1a receptor nucleic acid sequence, wherein the truncated sequence encodes inactive Bmpr1a polypeptide. A mutant HFSC also can contain an isolated inactive Bmpr1a receptor polypeptide (SEQ. ID. NO. 5), where Bmpr1a binding to BMP is substantially inhibited. In addition, a pre-excision mutant HFSC can contain a recombination site-flanked Bmpr1a nucleic acid sequence, such that administration of a recombination activator to the HFSC results in expression of an inactive Bmpr1a polypeptide. A mutant HFSC containing an antisense oligonucleotide that operatively hybridizes with a Bmpr1a mRNA sequence to inhibit intracellular translation of a Bmpr1a polypeptide is also part of the invention.

Mutant hair follicle (HF) cells containing the aforementioned Bmpr1a mutation can be selected from the following: a hair bulb (HB), dermal papilla (DP), dermal sheath (DS), precortex (PC), inner root sheath (IRS), outer root sheath (ORS), hair shaft (HS), bulge (Bu), arrector pili muscle (APM), and sebaceous gland (SG) cells. The mutant hair follicle cell containing the Bmpr1a mutation can be a resting, self-renewing, proliferating, transient amplifying, differentiating, or apoptotic cell. The mutant Bmpr1a gene can be inserted into the hair follicle stem cell by a method such as transfection with a vector, electroporesis, biolistic particle delivery, liposome encapsulation, micro-vessel encapsulation, particle bombardment, or microinjection.

In additional embodiments, the present invention relates to vectors, which include a Bmpr1a nucleic acid sequence, recombination sites, and a plasmid. The vectors are used to produce knockout organisms. The vectors can be used to promote an increase in the HFSC population in vitro or in vivo. Thus, the vector may comprise a promoter and a stem cell activator such a nucleic acid sequence encoding antisense Bmpr1a, P-PTEN, activated Akt, Noggin, or activated PI3K. Alternatively, the vector can contain a promoter, and a gene such as PTEN, Akt, GSK-3, cyclin D1, Tert, PI3K, Smad1,5,8, P27, and derived mutant genes. The invention includes hair follicle stem cells containing one of the foregoing vectors. A host organism comprising the hair follicle stem cell with the vector is also contemplated.

The vector, preferably is an inducible Cre expression vector, with Lox recombination sites flanking the target gene. The vector can include a target gene that is a sequence homologous to Wt Bmpr1a nucleic acid sequence so that when targeted recombination within the flanked Bmpr1a gene occurs, Bmpr1a gene activity is inhibited. Alternatively, the vector can include a Bmpr1a nucleic acid sequence, or derivative variant thereof. Additionally, the Flp recombination system can be used. As such, the method is initiated by forming a vector that contains the Bmpr1a-related sequence that is subsequently used to transfect embryonic stem cells. A host organism can be transfected with the vector containing a homologous Bmpr1a recombination sequence, and a regulatory element. This vector-mediated method for obtaining a Bmpr1a mutant organism will include use of the inducible Cre/Lox system, whereby the Bmpr1a gene is flanked by LoxP sites. In particular, mice can be transfected with this Bmpr1a vector to generate Bmpr1a mutations, wherein the Bmpr1a receptor is inactive. Specifically, pre-excision and post-excision Mx1-Cre⁺, Bmpr1a^{fx/fx} mice are formed using the vector. A Bmpr1a post-excision knockout mouse results, wherein Exon 2 of the Bmpr1a gene has been substantially eliminated through Cre recombinase-mediated excision of Exon 2. In this Bmpr1a post-excision knockout mouse, an inactive Bmpr1a receptor polypeptide is expressed, where binding to BMP is substantially inhibited.

The present invention relates to anti-Bmpr1a antibodies directed against the Wt and mutant Bmpr1a polypeptides, and fragments thereof. Also contemplated is a hair follicle stem cell (HFSC) comprising an isolated antibody, such as anti-Bmpr1a antibody, anti-BMP antibody, and fragments thereof, whereby the antibody induces hair follicle stem cell proliferation in vitro or in vivo by inhibiting BMP binding to Bmpr1a receptor.

In vitro hair follicle stem cell cultivation systems are within the invention's scope, wherein a hair follicle stem cell population proliferates. The cultivation system may possess an isolated hair follicle tissue section or an isolated hair follicle stem cell population with at least 104 cells in culture medium, and an isolated Noggin polypeptide that operably bind to Bmpr1a cell receptors, wherein Bmrp1a receptor binding to BMP is substantially inhibited. The isolated hair follicle tissue or HFSC population can be obtained from Wt or mutant Bmpr1a organisms. Alternatively, antibodies such as anti-Bmpr1a antibodies, anti-BMP antibodies, and fragments thereof, can be utilized in the in vitro hair follicle stem cell cultivation system to cause wild type stem cell proliferation. Additionally, mutant Bmpr1a stem cells may be cultivated in in vitro culture medium since the mutant stem cells comprise inactive Bmpr1a cell receptors that are unresponsive to inhibitory BMP signals.

In still an additional embodiment, methods for increasing hair follicle stem cell population numbers in vitro and in vivo are also within the scope of the invention. Methods include the following: formation of post-excision Mx1-Cre⁺ Bmpr1a^{fx/fx} knockout mutant organisms; formation of post-excision Mx1-Cre⁺ Bmpr1a^{fx/fx} Z/EG knockout mutant organisms; in vitro cultured Bmpr1a mutant hair follicle stem cells; in vitro cultured isolated hair follicle wild type and Bmpr1a mutant tissue; and in vitro cultivated wild type hair follicle stem cells, with either Bmpr1a antisense oligonucleotide, antibody (anti-Bmpr1a and anti-BMP), or Noggin activators.

### BRIEF DESCRIPTION OF DRAWINGS

FIG. 1 depicts a hair follicle (HF) structure, with expression patterns and cellular localization of Noggin;

FIG. 1A illustrates a schematic diagram of the HF structure, labeling structures in the cycling and permanent segments;

FIGS. 1B-1E depict hair follicle tissue section stains, with Masson's trichrome staining of the sebaceous gland (SG) and bulge (Bu) regions, which analyze Noggin expression patterns in the anagen and catagen phases using Noggin-LacZ knock-in mice;

FIG. 1B depicts bulge, sebaceous gland, and arrector pili muscle regions in anagen staining of Noggin-LacZ tissue;

FIG. 1C is a tissue section depicting bulge, sebaceous gland, and arrector pili muscle;

FIG. 1D depicts dermal papilla in catagen phase tissue in Noggin-LacZ tissue;

FIG. 1E depicts bulge and sebaceous gland in catagen tissue;

FIG. 1F depicts cellular-localization of Smad4 in HFs in early anagen;

FIG. 1G depicts cellular-localization of Smad4 in HFs in late anagen;

FIG. 1H depicts cellular-localization of Smad4 in HFs in telogen phases;

FIG. 1I illustrates a schematic diagram of the overlapped expression patterns of both Noggin and BMP4 in the anagen phase, wherein the blue regions depict Noggin activity, and the green regions depict BMP4 activity;

FIG. 1J depicts graphical depictions of relative expression levels of Noggin and BMP4, and the resultant BMP activity gradient is illustrated in FIG. 1J for anagen;

FIG. 1K depicts graphical depictions of relative expression levels of Noggin and BMP, and the resultant BMP activity gradient is illustrated for catagen phases;

FIG. 2 depicts analysis of the Mx1-Cre-dependent DNA recombination efficiency in the Z/EG reporter mice and comparison of hair loss and tumors between Wt and Bmpr1a mutant mice;

FIG. 2A depicts GFP expression patterns in the HFs and in the epidermis of the P2 induced Mx1-Cre Z/EG mice when analyzed on P15;

FIG. 2B depicts LacZ expression patterns in the HFs and in the epidermis of the P2 induced mice when analyzed on P15;

FIG. 2C depicts GFP expression patterns in the HFs and in the epidermis of the P2 induced mice when analyzed on P40;

FIG. 2D depicts LacZ expression patterns in the HFs and in the epidermis of the P2 induced mice when analyzed on P40;

FIGS. 2E-H depicts that expression of GFP is detected in only some of the HFs and the epidermis in the P4-induced reporter mice, consistent with recombination occurring in some, rather than all HFs;

FIG. 2E depicts GFP expression patterns in HFs when analyzed on P15;

FIG. 2F depicts LacZ expression patterns, where only some of the HFs and epidermis of the P4-induced reporter mice lack LacZ expression, and where loss of LacZ expression was seen throughout the HFs but not in the bulge area;

FIG. 2G depicts GFP expression patterns when analyzed on P40, where GFP expression was lost in the majority of HFs and the epidermis in the P4-induced mice;

FIG. 2H depicts LacZ expression patterns when analyzed on P40, while LacZ expression was detected in the majority of HFs and the epidermis;

FIGS. 2I-2J depict photographs of dorsal and ventral skin/hair of Wt, P2-, and P4-induced Bmpr1a mutant mice on P120, where mutant hair loss is depicted in comparison to Wt;

FIG. 2I depicts dorsal whole body P120 photographs for Wt, Bmpr1a mutant P2- and P4-mice respectively;

FIG. 2J depicts ventral P120 photographs for Wt, Bmpr1a mutant P2- and P4-mice respectively;

FIGS. 2K-2M depict photos of the subcutaneous surface of the ventral skin, for Wt, and P2-induced Bmpr1a mutant mice, where P90 and P180 photos, in 2L and 2M respectively, depict tumors that become progressively larger from the P90 to P180 time points, wherein:

FIG. 2K depicts the Wt ventral skin;

FIG. 2L depicts P2-induced Bmpr1a mutant skin on P90; and

FIG. 2M depicts P2-induced Bmpr1a mutant skin on P180;

FIG. 3 depicts histological analysis of hematoxylin-eosin (H&E)-stained hair follicles from sections of Wt, P2-, and P4-induced Bmpr1a mutant mice that were analyzed on P15, 20, 25, 30, and sections of HF tumors in the P2-induced mice analyzed on P90 or P180;

FIGS. 3A-3L depict histological sections of hair germ and follicle regions from the Wt control (FIGS. 3A-3D), P2-induced (FIGS. 3E-3H), and the P4-induced (FIGS. 3I-3L) Bmpr1a mutant mice, where aberrant hair follicle structures were observed in P2- and P4-induced mutant mice;

FIG. 3A depicts H&E staining of a Wt control HF section on P15;

FIG. 3B depicts H&E staining of a Wt control mouse HF section on P20;

FIG. 3C depicts H&E staining of a Wt control mouse HF section on P25;

FIG. 3D depicts H&E staining of a Wt control mouse HF section on P30;

FIG. 3E depicts H&E staining of a P2-induced mouse HF section on P15;

FIG. 3F depicts H&E staining of a P2-induced mouse HF section on P20;

FIG. 3G depicts H&E staining of a P2-induced mouse HF section on P25;

FIG. 3H depicts H&E staining of a P2-induced mouse HF section on P30;

FIG. 3I depicts H&E staining of a P4-induced mouse HF section on P15;

FIG. 3J depicts H&E staining of a P2-induced mouse HF section on P20;

FIG. 3K depicts H&E staining of a P2-induced mouse HF section on P25;

FIG. 3L depicts H&E staining of a P2-induced mouse HF section on P30;

FIGS. 3M-3T depict sections from a typical HF tumor with the features of matricomas are seen in the P2-induced Bmpr1a mutant mice at P90 and P180;

FIG. 3M depicts a section of HF tumors at P90;

FIG. 3N depicts a section of HF tumors at P180;

FIG. 3O depicts a section of a large solid HF tumor;

FIG. 3P illustrates melanin deposits in HF tumors;

FIG. 3Q depicts cyst, “bulb-like” structures, and sebaceous gland structures;

FIG. 3R depicts low magnification of multiple HF tumors;

FIG. 3S depicts HF tumors;

FIG. 3T depicts the presence of keratin in HF tumors;

FIG. 4 depicts immunohistochemical analyses of sections derived from Wt and Bmpr1a mutant mice using different differentiation markers (AE 13, 15, CK5, 10, 14) detected by AEC staining (red), with hematoxylin (blue) counterstaining;

FIG. 4A depicts that AE13 is detected in the hair shaft and precortex cells of Wt control HFs;

FIG. 4B depicts that AE13 also appears in the center of the cyst in the abnormal HFs of mutant mice;

FIG. 4C depicts that CK10 was detected in hair shaft and epidermis cells of Wt HFs;

FIG. 4D depicts that CK10 is barely detectable in the “bulb”-like structures in mutant HFs;

FIG. 4E depicts that AE15 is detected in the inner root sheath (IRS) cells of Wt HFs;

FIG. 4F depicts that AE15 exists in the subset of the inner layer of each “bulb”-like structure of mutant HFs;

FIG. 4G depicts that CK5 is detected in the outer root sheath (ORS) and the epidermis cells of Wt;

FIG. 4H depicts that CK5 is also located in the cells of ORS-like and the cyst boundary in mutant mice;

FIG. 4I depicts that CK14 is detected in cells of ORS and the epidermis of Wt HFs;

FIG. 4J depicts that CK14 also appears in the cyst boundary of abnormal HFs of mutant mice;

FIG. 5 depicts analyses of HFs of Wt and Bmpr1a mutant mice using proliferation markers (Ki67, BrdU, β1-integrin) detected by AEC (red) labeling and hematoxylin (blue) counterstaining in HF and tumors from skin sections;

FIG. 5A depicts that the BrdU-LTR cells are found occasionally in the bulge of Wt HFs;

FIG. 5B depicts that the BrdU-LTR cells are significantly increased in the bulge area of abnormal HFs from Bmpr1a mutant mice;

FIG. 5C depicts that with a three-hour post-BrdU pulse, labeled cells are located in the bulge and the bottom region of HB in Wt HFs;

FIG. 5D depicts that after a three-hour post-BrdU pulse, labeled cells are located in the base of the “bulb”-like structures of tumorous HFs in Bmpr1a mutant mice;

FIG. 5E depicts that with a three-day post-BrdU pulse, labeled cells are located in the bulge and are migrating to the HM region in Wt HFs;

FIG. 5F depicts that after a three-day post-BrdU pulse, labeled cells are located in the “bulb”-like structures of tumorous HFs in Bmpr1a mutant mice;

FIG. 5G-J depicts results of three days post-BrdU pulse labeling, and staining with Ki67 and β1-integrin markers in normal hair follicle and tumor sections;

FIG. 6 depicts analyses of expression and cellular localization of candidate molecules of the BMP and Wnt signaling pathways (i.e., Tcf3, β-catenin, Lef-1, PTEN, Akt, GSK3β) in Wt and tumorous skin sections;

FIG. 6A depicts Tcf3 and β-catenin staining of a Wt control section, where bulge staining is highlighted;

FIG. 6B depicts Tcf3 staining of a Wt control section;

FIG. 6C depicts β-catenin staining of a Wt control section;

FIG. 6D depicts Tcf3 staining of a Bmpr1a mutant mouse section;

FIG. 6E depicts Tcf3, β-catenin, Lef-1 staining of a Wt control section;

FIG. 6F depicts β-catenin staining of a Bmpr1a mutant mouse section;

FIG. 6G depicts Lef-1 staining of a Wt control section;

FIG. 6H depicts Lef-1 staining of a Bmpr1a mutant mouse section;

FIG. 6I depicts inactivated PTEN staining of a Wt control section;

FIG. 6J depicts inactivated PTEN staining of a Bmpr1a mutant mouse section, also depicting a cyst;

FIG. 6K depicts BrdU-LTR and AktS473 staining of a Wt control section;

FIG. 6L depicts a diagram of the photo in FIG. 6K depicting BrdU-LTR and AktS473 staining and corresponding arrested and activated stem cells;

FIG. 6M depicts AktS473 staining of a Bmpr1a mutant mouse section, depicting a cyst region;

FIG. 6N depicts inactivated GSK3β staining of a Wt control section;

FIG. 6O depicts inactivated GSK3β staining of a Bmpr1a mutant mouse section;

FIG. 7 depicts the proposed BMP-dependent pathway and the role of BMP signaling involvement in HF development and tumorigenesis in Bmpr1a mutant in comparison to Wt animals;

FIG. 7A depicts a diagram of the stem cell zone, with interaction of BMP with Bmpr1a, where β-catenin enters the nucleus, and Tcf3 also exists in the nucleus;

FIG. 7B depicts a diagram of the differentiation zone, where Lef-1 and β-catenin enter and co-exist in the nucleus;

FIG. 7C depicts a diagram of the HF in the Wt control, where arrested stem cells from the Bulge become activated stem cells and transient amplifying cells, progressing in differentiation and proliferation zones;

FIG. 7D depicts a diagram of the HF in the Bmpr1a mutant knockout mouse, where a number of proliferating cells are produced along with multiple crypt regions;

FIG. 8 depicts analyses of hair follicle expression pattern distributions for BMP4, Noggin, and Bmpr1a, where sections of BMP4-LacZ and Noggin-LacZ knockin mice were stained with β-galactosidase;

FIG. 8A depicts BMP4 and LacZ staining of a HF longitudinal section, with HF structures IRS, precortex (PC), hair matrix (HM), and DP;

FIG. 8B depicts a diagram of the BMP4 zone in the HF structure;

FIG. 8C depicts Noggin and LacZ staining of a HF longitudinal section, with HF structures SG, BU, DP, and dermal sheath;

FIG. 8D depicts a diagram of the Noggin expression pattern in the HF structure, where Noggin is prominent in the DP region and the dermal sheath along the bottom of the hair bulb in anagen;

FIG. 8E depicts expression of Bmpr1a throughout the entire HF region, including the bulge area;

FIG. 8F depicts a diagram of the Bmpr1a expression pattern in the HF structure;

FIGS. 9A-9C depict analysis of expression and cellular cytoplasmic localization of Smad4 with DAPI counterstain in skin sections derived from the Bmpr1a mutant mice, wherein DAPI stains nuclei, with little or no cytoplasmic staining;

FIG. 9A depicts Smad4 stain alone;

FIG. 9B depicts DAPI stain alone; and

FIG. 9C depicts Smad4 and DAPI staining together.

### DETAILED DESCRIPTION

The present invention is based upon the discovery that BMP signaling plays an essential role in the determination of HFSC fate, promoting epidermal but inhibiting hair follicle lineage commitment. This is, at least partially, through antagonizing the β-catenin activity through the Pten-Akt cascade. It has been discovered, as detailed in the examples, that blocking the BMP signal by removal of its BMP-receptor type IA (Bmpr1a) leads to HFSC expansion, disruption of hair shaft differentiation, and eventually the formation of matricomas. In contrast, overexpression of BMP4 results in overproduction of epidermal stem cells, the subsequent epithelial hyperplasia, and inhibition of hair follicle development. The current invention utilizes this novel discovery and provides methods, compositions, cells, tissues, organisms, cultivation systems, and kits that may be employed to control HFSC fate determination.

Crosstalk Between the Wnt and BMP Signal Transduction Pathways Control HFSC Fate Determination

One aspect of the invention, therefore, is directed toward methods and compositions to regulate HFSC fate by modulating key regulatory molecules in either one of or both of the Wtn or BMP signal transduction pathways. Briefly outlined below are the key regulatory elements in each pathway and the mechanism by which these elements influence HFSC fate.

The Wnt pathway has been well characterized and has been shown to play an essential role in regulating proliferation and differentiation of HFSC. Among the key regulators in this system is β-catenin. Constitutive expression of β-catenin results in de novo hair follicle morphogenesis in adult skin. Targeted inactivation of β-catenin diverts stem cells toward an epidermal fate. Lef-1, a partner of β-catenin, is not only required for the early induction of the hair germ, but it is essential for hair shaft differentiation. Enforced expression of a mutant Lef-1, which lacks the β-catenin binding domain, leads to squamous cysts and skin tumors. Tcf3, another β-catenin partner, plays a role in the maintenance of HFSC.

The BMP signal pathway also plays a pivotal role in morphogenesis and postnatal regeneration of the hair follicle. Neutralizing BMP activity by overexpression of Noggin, an antagonist of BMPs, leads to the induction of the anagen phase and disruption of hair shaft differentiation. Targeted inactivation of Noggin causes significant retardation of hair follicle induction, and increased proliferation of basal epidermal keratinocytes. Mice with ectopic expression of BMP4 in hair follicles depict a complete deficiency of hair growth after the first growth cycle, leading to progressive balding. Moreover, long-term treatment of mice with BMP2 or BMP4 leads to thickening of the epidermis with development of psoriasis.

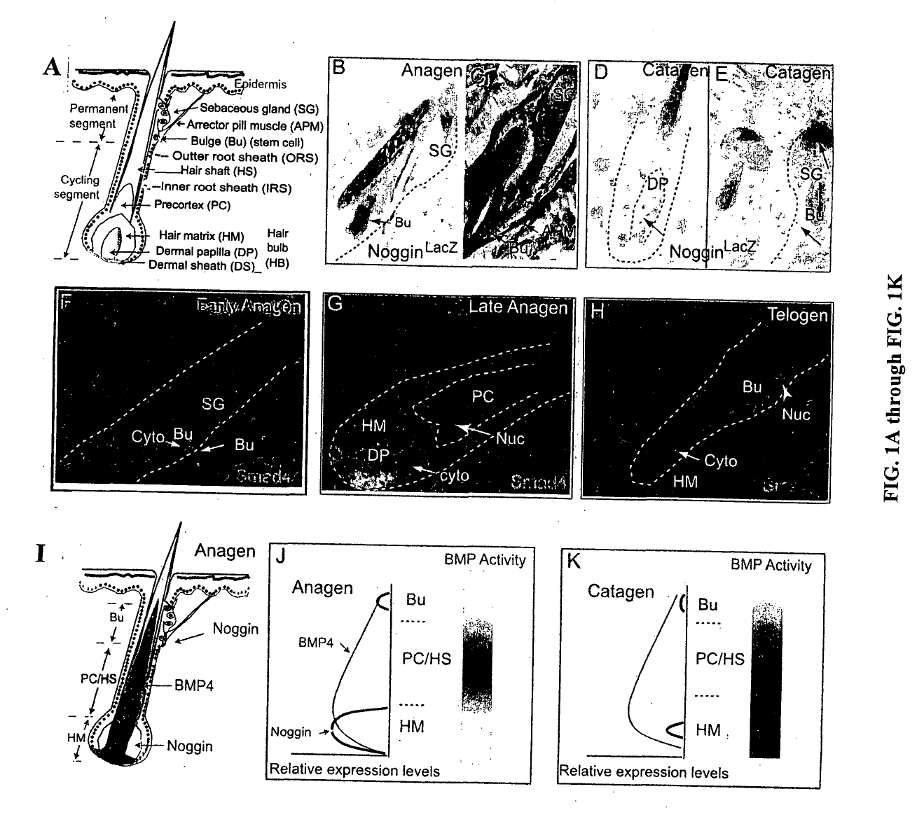

Both the BMP and Wnt pathways control HFSC development. But until the present discovery, the crosstalk between these two pathways to control HFSC fate was not elucidated. Significantly, it is established herein that the BMP signal controls the HFSC number by restricting activation and expansion of stem cells in homeostasis and regeneration. The Noggin signal overrides the BMP activity, which causes a cascade of the PTEN-PI3K-Akt-GSK3β pathway. Noggin interaction with the Bmpr1a receptor on HFSCs results in the translocation of β-catenin from the cytoplasm into the nucleus of the arrested stem cell, thereby activating stem cell division. Bmpr1a receptor inactivation, on the other hand, results in blocking hair follicle epithelial cells from sensing the BMP signal, which in turn generates an increase in the number of long-term (arrested) HFSCs. In addition, this impaired differentiation and resistance to apoptosis occurred, eventually leading to the formation of profuse hair follicle tumors. The BMP signal distribution pattern, which co-exists with a Noggin-dependent activity gradient along the hair follicle villus axis, plays a critical role in the control of the number of the hair follicle stem cells by restricting activation and expansion of hair follicle stem cells. Moreover, the BMP signal defined specified zones within the hair follicle, as shown diagrammatically in FIG. 1I, in which HFSCs proceed through self-renewal, proliferation, differentiation, and apoptosis.

Nucleotide Sequences and Polypeptide Sequences to Control HFSC Fate

One aspect of the invention encompasses the use of an isolated nucleotide sequence that may be employed to control HFSC fate based upon the pathways detailed above. By way of non limiting example, BMP, Noggin, PTEN, PI3K, Akt, GSK3, TCF3, or any other PTEN pathway genes, nucleotide sequences, or polypeptides, for example, can be utilized to alter cell proliferation, differentiation, and apoptosis in hair follicle cells. A sequence from this group may be utilized, by way of example, to form a knockout or mutant organism

In one embodiment, the gene is an isolated Wt Bmpr1a gene. The Wt Bmpr1a gene encodes a functional Bmpr1a receptor that can operatively bind to BMP. The Bmpr1a gene may be obtained from cell line XC131 Protein Accession No. XP_{—}017633. The Bmpr1a gene is located on chromosome, locus 10q22.3 in mice; and the human homolog LOC88582 of Bmpr1a is located on Human Chromosome: '6. Homologs from other mammals and other organisms are also included within the scope of the present invention. While it is preferred to isolate a gene, other hereditary units may be used. Moreover, the isolated sequence can be any of a variety of structures, in addition to a gene, such as gene fragments, polynucleotides, oligonucleotides, and any nucleotide structure that can be substituted into the genome of a host and result in expression of a functional Bmpr1a polypeptide, until it is desired to mutagenize such a genomic structure. Homologous sequences are available, as are orthologs. Functional mutant sequences of Bmpr1a may be used. Gene fragments are available, as long as the organism properly develops prior to activation of the mutant.

In one alternative of this embodiment, a Bmpr1a nucleotide sequence is utilized. The human Bmpr1a nucleotide sequence is SEQ ID NO. 8, which encodes the human Bmpr1a polypeptide as SEQ ID NO. 7, is suitable for use in a number of embodiments of the present invention. In a further embodiment, a murine nucleotide sequence, such as SEQ ID NO. 1 may be utilized. In still additional embodiments, mutant or truncated nucleotide sequences may be suitable. Exon 2 of the murine Wt Bmpr1a gene contains nucleotides 68 through 230 of the gene's coding region, as shown in SEQ ID NO. 3. This Bmpr1a nucleic acid sequence encodes a region extending from the 23^{rd} amino acid (glycine) through the 77^{th} amino acid (isoleucine) of the Wt Bmpr1a polypeptide chain, as presented in SEQ ID NO. 6. The mutant Bmpr1a gene lacking Exon 2 is exhibited in SEQ ID NO. 2, with the truncated mutant Bmpr1a polypeptide is presented in SEQ ID NO. 5. Any of these sequences, as will be readily appreciated by a skilled artisan, may be suitable for use depending upon the embodiment.

The invention also encompasses the use of nucleotide sequences other than the sequences specifically set forth herein to the extent that the sequence encodes a polypeptide having the structure and function described herein. Typically, these nucleotide sequences will hybridize under stringent hybridization conditions (as defined herein) to all or a portion of a nucleotide sequence detailed above or its complement. The hybridizing portion of the hybridizing nucleic acids is usually at least 15 (e.g., 20, 25, 30, or 50) nucleotides in length. The hybridizing portion of the hybridizing nucleic acid is at least 80%, preferably, at least 90%, and is more preferably, at least 95% identical to the sequence of a portion or all of a target nucleic acid sequence.

Hybridization of the oligionucleotide probe to a nucleic acid sample is typically performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming at 1% mismatching results in a 1° C. decrease in the Tm, the temperature of the final wash in the hybridization reaction is reduced accordingly. For example, if sequences have greater than 95% identity with the probe is sought, the final temperature is approximately decreased by 5° C. In practice, the change in Tm can be between 0.5 and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and target nucleotide sequence. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

In a typical embodiment, the nucleotide sequence selected will preferably be isolated from the homologous species in which the nucleotide sequence is to be used. For example, if the procedure is to be conducted in a mouse, then the Bmpr1a gene is preferably isolated from a mouse. Any of a variety of species, however, may be used. Preferably, however, eukaryotic organisms are used. It is more preferred to use a mammalian gene, in particular mus musculus (mouse).

In another aspect of the invention, the Bmpr1a nucleotide sequence can be mutagenized and rendered nonfunctional. Alternatively, at least two recombination sites can flank all or a portion of the Wt Bmpr1a nucleotide sequence. A mutation is made in the Wt Bmpr1a gene or nucleotide sequence, such that the sequence encodes an inactive Bmpr1a receptor polypeptide that is unable to bind with BMP. The resultant mutation can be a frame shift, point, substitution, or deletion mutation. Importantly, the mutant Bmpr1a sequence should remain substantially homologous to the Wt, but render the resultant gene nonfunctional. A preferred option is to form a mutant Bmpr1a sequence that is a truncated sequence, which is a shortened sequence that encodes a nonfunctional Bmpr1a receptor polypeptide molecule. It is most preferred to knockout Exon 2 of the sequence, resulting in a truncated nonfunctional Bmpr1a gene sequence, such as SEQ ID NO.2. As such, a deletion mutation may be made directly in the sequence.

Alternatively, if a conditional mutant is to be formed, the Bmpr1a nucleic acid sequence should be such that it is fully functional throughout the development of the organism and until steps are taken to inactivate the nucleotide sequence. Inactivation occurs once the organism has sufficiently developed. Conditional mutant formation is accomplished by placing nucleotide sequences flanked by recombination sequences into the genome so that recombination can be later activated. The recombination sequence can be used to cleave a gene or exon from the genome. Preferably, a pair of recombination fragments is used. Recombination can be accomplished by inserting the sequence in a vector that places recombination sites on either end of the targeted nucleotide sequence to be excised. The recombination sites are substituted with the nucleotide sequence into the organism, with the recombination sites activated at a later time.

Vectors

In another aspect of the invention, depending upon the embodiment, a nucleotide sequence detailed above may be inserted into a vector. An “appropriate vector” is typically a vector that contains the necessary elements for transcriptional and translational control of the inserted coding sequence in a suitable host. These elements generally will include regulatory sequences, such as enhancers, constitutive and inducible promoters, and 5′ and 3′ untranslated regions in the vector and in polynucleotide sequences encoding a target polypeptide. Such elements may vary in their strength and specificity. Specific initiation signals may also be used to achieve more efficient translation of nucleotide sequences encoding a target polypeptide. These signals, for example, include the ATG initiation codon and adjacent sequences (e.g. the Kozak sequence). In cases where nucleotide sequences encoding the subject polypeptide and its initiation codon and upstream regulatory sequences are inserted into the appropriate expression vector, no additional transcriptional or translational control signals may be needed. But in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (See, e.g., Scharf, D. et al. (1994) Results Probl. Cell Differ. 20:125-162).

Methods that are well known to those skilled in the art may be used to construct expression vectors containing a desired sequence and appropriate transcriptional and translational control elements. These methods include, for example, in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16).

Depending upon the embodiment, either a conditional recombination sequence or mutant sequence may be inserted into a vector. The vector for forming the conditional mutant will include the targeted Bmpr1a nucleic acid sequence, preferably flanked by recombination sites for the conditional sequence. The conditional vector is structured such that the targeted, recombination-site flanked gene or nucleotide sequence will be cut from the genome to form a knockout mutant.

Alternatively, a mutated Bmpr1a gene or sequence in a vector may be directly substituted for the Wt to render a Bmpr1a gene nonfunctional. Substitution, deletion, loss of function, and frame shift mutations are examples of mutant Bmpr1a sequences that result in the nonfunctional gene. Regardless of the mutant formed, the Wt Bmpr1a gene sequence found in a selected host organism may be substantially eliminated or made nonfunctional through insertion of the vector's mutant Bmpr1a nucleic acid sequence. SEQ ID NO. 2 is an example of a suitable mutated Bmpr1a sequence that can be used in a recombination vector to obtain the Bmpr1a mutant organism. The truncated, inactive mutant Bmpr1a polypeptide of SEQ ID NO. 5 is encoded by the truncated mutant nucleic acid sequence of SEQ ID NO. 2.

Depending upon the embodiment, either eukaryotic or prokaryotic vectors may be used. Suitable eukaryotic vectors that may be used include MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pCEP4, and pEBVHis vectors. The MSCV or Harvey murine sarcoma virus is preferred. Suitable prokaryotic vectors that can be used in the present invention include pET, pET28, pcDNA3.1/V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT vectors.

A variety of selectable markers may optionally be included with the vector. Available markers include antibiotic resistance genes, a tRNA gene, auxotrophic genes, toxic genes, phenotypic markers, colorimetric markers, antisense oligonucleotides, restriction endonuclease, enzyme cleavage sites, protein binding sites, and immunoglobulin binding sites. Specific selectable markers available include LacZ, neo, Fc, DIG, Myc, and FLAG.

The conditional vector may be used to transfect any of a variety of cells. It is preferred to transfect embryonic stem cells (ES). Typically, the ES will be transplanted into the uterus of an adoptive host mother, so that an embryo can gestate from the ES, with the recombination sequence ultimately present in HFSCs. The vector could also be used to transfect HFSC in a mature organism, such as an embryo. The particular type of cell to be transfected will influence the vector selected. Also, the cells to be transfected can be grown in vivo or in vitro. The mutant sequence can be used to transfect isolated HFSCs or cells present in an embryo or mature organism.

The conditional vector may optionally include recombination sites that cause insertion of a conditional knockout mutation (Bmpr1a^{fx/fx}, for example) or a mutant, wherein Bmpr1a is rendered nonfunctional. Formation of a conditional transgenic Bmpr1a knockout organism is preferred. This can be achieved by the knock-in of a Cre or Flp recombinase site, or a Cre-Flp site combination thereof, into a specific Bmpr1a gene locus or loci. The expression of Cre or Flp recombinase will be under the control of the endogenous locus in a tissue-specific, time-dependent manner. The temporal/spatial-restricted Cre/Flp expression line will lead to a conditional or selective deletion of the target gene (e.g., Bmpr1a) when crossed with an organism in which LoxP or FRT recombination sites flank the target gene. Preferably, LacZ and GFP markers, flanked by LoxP or FRT recombination sites, may be utilized to determine the efficiency of recombination of the target gene. A combination of the Cre/LoxP and Flp/FRT systems will also allow selective and simultaneous deletion of two gene loci of interest. Other alternative recombination systems and marker systems, however, can be devised and used as known in the art.

The two functional units required for in vivo targeted conditional DNA deletion of the Bmpr1a receptor gene in the Cre-LoxP organism system are: (1) expression of the P1 Cre recombinase gene, often induced by a cell-specific or regulated promoter; and (2) at least one integrated DNA target gene segment that is flanked by LoxP, a 34 bp P1 DNA sequence. The LoxP-flanked target DNA is said to be “floxed.” The Cre/LoxP system is a tool for conditional time-specific and tissue-specific postnatal knockout of selected target genes (e.g., Bmpr1a), which cannot be investigated in conventional gene knockout animals, such as mice, because of the nonfunctional target gene's early embryonic lethality.

Thus, a Bmpr1a gene is isolated, and a modified Bmpr1a gene is made by insertion of Lox recombination sites and marker sites into the gene. A Bmpr1a vector is made by insertion of the modified Bmpr1a gene into a vector. An embryonic stem (ES) cell is then transfected with the Bmpr1a vector to form a Bmpr1a embryonic stem cell. The Bmpr1a embryonic stem cell is implanted into a host uterus to form a Bmpr1a^{fx/fx} organism. The foregoing method can be modified, wherein Bmpr1a vector formation involves insertion of Lox recombination sites flanking Exon 2 of the Bmpr1a gene and insertion of marker sites into the vector's genomic sequence. Another method of modification utilizes a mutant Bmpr1a nucleic acid sequence, which can be administered to the ES cell by methods including, but not limited to, electroporation, microinjection, micro-vessel transfer, particle bombardment, and liposome mediated transfer.

It is also contemplated that a variety of expression vector/host systems may be utilized to contain and express nucleotide sequences encoding polypeptides of the invention. By way of non limiting example, these include microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355). In additional embodiments, expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, L M. and N. Somia (1997) Nature 389:239-242).

Formation of Mutant Organisms

Another aspect of the invention encompasses the formation of a mutant non human organism. In one embodiment, a conditional Bmpr1a mutant HFSCs is formed by transfecting embryonic stem cells, with the Bmpr1a gene later rendered nonfunctional upon activation in a postnatal organism. The conditional gene mutation in a pre-recombination organism is maintained dormant throughout gestation. The conditional Bmpr1a mutant cells can be formed in vivo, such as in an Mx1-Cre organism. Alternatively, Wt HFSCs can be isolated and treated in vitro to obtain Bmpr1a mutant HFSCs. A vector can be utilized to create a Bmpr1a gene recombination-induced conditional mutation in HFSCs. In yet another embodiment, a Bmpr1a gene mutation can be directly induced in HFSCs by a mutagen. The conditional mutant HFSCs can be studied and used as tools to better understand HFSCs and the pathways influencing HFSC differentiation, proliferation, and apoptosis. The conditional knockout cells and organisms include pre-recombination and post-recombination cells and organisms. A post-recombination Bmpr1a mutant organism contains cells with inactive Bmpr1a receptors.

Any of the eukaryotic vectors detailed herein can be used to transfect eukaryotic host cells including mammalian, amphibian, or insect cells, eukaryotic cells include human, mouse, and frog cells. The preferred process includes transfecting an embryonic stem cell of a selected species with the vector. The transfected embryonic stem cell is then transplanted into an adopted host mother. The embryonic stem cell will gestate to an embryo followed by birth of a conditional mutant organism. Thus, mutant offspring are formed, such as a Bmpr1a^{fx/fx} mutant organism. Specific conditionally active mutants include HFSCs. It is likely that specific host cells that are transfected include hair follicle cells including HFSCs, a hair bulb (HB), dermal papilla (DP), dermal sheath (DS), precortex (PC), inner root sheath (IRS), outer root sheath (ORS), hair shaft (HS), bulge (Bu), arrector pili muscle (APM), and sebaceous gland (SG).

Typically, two organism lines (mouse, for example) are required for formation of a conditional gene deletion organism: a conventional transgenic line with, for example, Cre-targeted to a specific tissue or cell type, and a strain that embodies a target gene (endogenous gene or transgene) flanked by two recombination (LoxP, for example) sites in a direct orientation (“floxed gene”). When the target gene is the Bmpr1a gene, recombination occurs by excision and, consequently, inactivation of the floxed Bmpr1a target gene. Since recombination and Bmpr1a gene excision occurs only in those cells expressing Cre recombinase, the Bmpr1a target gene remains active in all cells and tissues that do not express Cre recombinase. Gene excision is induced by a recombination activator, such as PolyI:C or interferon, which in turn triggers Cre recombinase expression. The recombination activator is preferably injected postnatally to ensure organism survival. Most preferably the recombination activator is injected at 0, 1, 2, or 20 days after birth, or anytime thereafter. Cre and FLP recombinase are exemplary recombinases that may be used. Cre recombinase is used to cleave Lox sites flanking the Bmpr1a gene, such as LoxP and LoxC2 sites. Alternatively, FLP recombinase can be used with FRT recombination sites flanking the Bmpr1a gene.

By way of non limiting example, Mx1-Cre⁺ and Bmpr1a^{fx/fx} mice progeny are crossed to form a conditional mouse mutant Mx1-Cre⁺ Bmpr1a^{fx/fx}. This organism can be conditionally mutated after birth to cause formation of tumors, including matricomas in the hair follicle. Once activated and mutated, an inactive Bmpr1a receptor polypeptide is expressed. An inactive HFSC containing a truncated Bmpr1a receptor polypeptide is formed, wherein BMP interaction is blocked. Any of a variety of recombination site-flanked Bmpr1a nucleic acid sequences can be knocked out and expressed. Flanking Bmpr1a recombination sites included in the present invention are Lox, LoxP and FRT sites.

The knockout organism permits conditional excision of the target Bmpr1a gene upon the injection of a recombination activator into the organism. The knockout animal may be a pre-recombination or post-recombination animal, where the pre-recombination animal is the Bmpr1a mutant animal prior to injection of the recombination activator and the post-recombination animal is the Bmpr1a mutant animal after injection of the activator.

In still another embodiment, a Bmpr1a^{fx/fx} and Bmpr1a^{fx/fx} Z/EG knockout mutant organism may be formed. These organisms are useful in characterizing a mutant phenotypic change in a hair follicle cell in vivo in the organism. The characterized phenotypic change can be the presence of increased HFSC population number, differentiation change, reduced apoptosis, and/or hair follicle tumorigenesis.

In yet another embodiment, a Mx1-Cre⁺ Bmpr1a^{fx/fx} Z/EG knockout mutant organism is formed. A pre-recombination Mx1-Cre⁺ Bmpr1a^{fx/fx} Z/EG knockout mutant organism for use in studying a hair follicle cell can be formed. The Mx1-Cre Lox Bmpr1a^{fx/fx} organism, obtained utilizing the previously described method, is crossed with a Z/EG organism to form a pre-excision hybrid Mx1-Cre Lox Bmpr1a^{fx/fx} Z/EG organism. Finally, a recombination activator is administered to the hybrid Mx1-Cre Lox Bmpr1a^{fx/fx} organism crossed with a Z/EG organism to induce Cre-mediated Lox site-directed intracellular Bmpr1a gene recombination. The post-recombination Mx1-Cre⁺ Bmpr1a^{fx/fx} Z/EG knockout mutant organism can be utilized to assess the efficacy of the recombination procedure in yielding hair follicle cells with the excised Bmpr1a gene encoding the inactive Bmpr1a receptor. The efficiency of the Bmpr1a gene recombination process is monitored by the detection of LacZ or GFP gene marker expression in hair follicle tissue and cells.

A number of suitable operative recombination activators may be utilized in the current invention. For example, operative recombination activators can include PolyI:C, interferon, or other interferon inducers. PolyI:C is a preferred recombination activator. The recombination activator induces Cre recombinase expression, which in turn results in excision of the Lox-flanked Bmpr1a nucleic acid sequence in cells of the mutant Bmpr1a organism. Preferably, Exon 2 of Bmpr1a is excised, rendering the Bmpr1a gene nonfunctional.

In the hair follicle tissue of the transfected animal, the resultant mutant Bmpr1a hair follicle cell contains a conditional mutant Bmpr1a gene that can encode an inactive Bmpr1a polypeptide. Alternatively, the cells can be mutagenized and nonconditional. The Bmpr1a mutant hair follicle cell can be a hair follicle epithelial stem (HFSC), resting, self-renewing, proliferating, transient amplifying, differentiating, tumor, or apoptotic cell. The mutant hair follicle cell may be made in vivo or in vitro by methods such as knockout organism formation, vector transfection, micro-vessel transfer, biolistic particle delivery, liposome-mediated transfer, electroporation, or microinjection of the Bmpr1a mutant gene. The Bmpr1a mutant hair follicle region is situated adjacent to the epidermis region. The hair follicle tissue can be isolated and transfected. Also, cells can be isolated and transfected.

Mutant Hair Follicle Cells and Tissues

Yet another aspect of the invention encompasses mutant hair follicle cells and tissue. In several embodiments, a mutant hair follicle cell having an inactive Bmpr1a receptor polypeptide is formed by activating the recombinase in the knockout organism as herein described. The mutant hair follicle cell's Bmpr1a binding to BMP is substantially inhibited. In particular, the mutant hair follicle cell can include the inactive Bmpr1a receptor polypeptide that is a truncated or a shortened Bmpr1a receptor polypeptide, such as the shortened Bmpr1a receptor polypeptide of SEQ ID NO. 5. This truncated Bmpr1a receptor polypeptide is encoded by a truncated, nonfunctional Bmpr1a gene (SEQ ID NO. 2) in which Exon 2 has been excised (SEQ ID NO. 3), as detailed above. This mutant Bmpr1a hair follicle cell either possesses an inactive Bmpr1a polypeptide or lacks the Bmpr1a polypeptide completely. Preferably, the mutant Bmpr1a hair follicle cell is a hair follicle stem cell (HFSC).

Because the expressed mutational changes can be clonal and expressed throughout the hair follicle, the Bmpr1a mutant hair follicle cell includes hair follicle stem (HFSC), resting, self-renewing, proliferating, transient amplifying, differentiating, a hair bulb (HB), dermal papilla (DP), dermal sheath (DS), precortex (PC), inner root sheath (IRS), outer root sheath (ORS), hair shaft (HS), bulge (Bu), arrector pili muscle (APM), and sebaceous gland (SG), tumor, and apoptotic cells. The mutant hair follicle cell can be located in the knockout organism or in isolated hair follicle tissue placed in vitro.

In some embodiments, HFSCs can be transfected with Bmpr1a-derived nucleic acid-containing vectors to obtain mutant Bmpr1a receptor-defective HFSCs. In the mutant Bmpr1a gene containing HFSC, the Bmpr1a receptor polypeptide binding to a bone morphogenic protein (BMP) is substantially inhibited. In particular, a mutant HFSC that possesses mutant Bmpr1a receptor DNA and/or RNA sequences that encode inactive Bmpr1a receptor polypeptides is part of the invention. The resultant Bmpr1a mutant HFSC can possess the properties of increased self-renewal or proliferation activity. A Bmpr1a mutant HFSC possesses the potential to transform into a hair follicle tumor, preferably a trifolliculoma.

In still other embodiments, the HFSC can possess an isolated Bmpr1a antisense fragment or antisense oligonucleotide that exists intracellularly, wherein the antisense fragment induces HFSC proliferation by inhibiting translation of Bmpr1a receptor polypeptide (SEQ ID NO. 4). The antisense fragment can be inserted into the HFSC by methods including, but not limited to, electroporation, transfection, microinjection, micro-vessel transfer, particle bombardment, and liposome mediated transfer. The isolated Bmpr1a antisense fragment can be synthesized and multiple copies generated in vitro using a sense template, as is known in the art. The antisense fragments, inserted into HFSCs, induce HFSC proliferation by inhibiting translation of the Bmpr1a receptor polypeptide. As such, HFSCs containing antisense fragments are contemplated.

Alternatively, Noggin polypeptide can be used to competitively bind to Bmpr1a receptor that in turn affects HFSC expansion and commitment. In particular, an isolated Noggin activator (Noggin polypeptide), or fragments thereof can be used. The Noggin activator acts to induce HFSC proliferation in vitro by inhibiting BMP binding to Bmrp1a receptor (SEQ ID NO. 4). Noggin's binding affinity for Bmpr1a receptor can be greater than BMP's affinity for the receptor. Noggin can be used in cells, tissue, or organisms, containing the same conditional or mutant Bmpr1a. Noggin can be provided by using a vector to express increased amounts of Noggin. The vector will typically locate in the cytoplasm and “flood” the cell with Noggin polypeptide. In this case, the cell will secrete Noggin into the intracellular mileau to bind to Bmpr1a receptor on HFSCs. Another option is to contact the cell, tissue, or organism with increased amounts of Noggin polypeptide. Wt hair follicle tissue can be exposed to a stem cell activator, such as Noggin, and cultivated in culture medium in vitro. The Noggin can be contained in beads, particles, or liposomes. Preferably, Noggin-beads are injected into the hair follicle containing tissue, placing the Noggin in contact with the HFSCs. Alternative activators could be used, such as members of the PTEN pathway.

In an alternative embodiment, an antibody to a polypeptide can be used to generate phenotypic changes in a host organism. In one embodiment, for example, the antibody will to bind specifically to Bmpr1a or BMP polypeptides and as such, will prevent the functioning of the Bmpr1a or BMP polypeptides. This, in turn, will result in an increase in the HFSC population in vivo or in vitro.

Alternatively, an antibody to a mutant Bmpr1a polypeptide may also be used to detect and monitor the presence of mutant Bmpr1a in hair follicle cells. Thus, isolated antibodies, such as anti-Bmpr1a antibody, anti-BMP antibody, and fragments thereof, where the antibody, acting as an hair follicle stem cell (HFSC) activator, induces HFSC proliferation in vitro by inhibiting BMP binding to Bmpr1a receptor can be used. Anti-Bmpr1a antibodies and anti-BMP antibodies are made, isolated, and administered to an HFSC population in vitro to disrupt BMP-mediated regulation. The binding of Bmpr1a receptor to BMP polypeptide is inhibited by the binding of either anti-Bmpr1a antibody or anti-BMP antibody to the HFSC population. This will cause the HFSC population to be expanded in vitro. Administration of the isolated antibodies to the HFSC population may occur by injection, liposome encapsulation, diffusion, or micro-vessel encapsulation. Antibodies can be obtained by polyclonal or monoclonal methodologies known to those in the art.

Antibodies to a target polypeptide may also be generated using methods that are well known in the art. Such antibodies may include, but are not limited to, polyclonal, monoclonal, chimeric, and single chain antibodies, Fab fragments, and fragments produced by a Fab expression library.

For the production of antibodies, various hosts including goats, rabbits, rats, mice, humans, and others may be immunized by injection with a target polypeptide that has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to a target polypeptide have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of a target polypeptide amino acids may be fused with those of another protein, such as KLH, and antibodies to the chimeric molecule may be produced.

Monoclonal antibodies to a target polypeptide may be prepared using a technique that provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-45). Alternatively, techniques described for the production of single chain antibodies may be adapted, using methods known in the art, to produce a target polypeptide-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the target polypeptide and its specific antibody. A two-site, monoclonal-based immunoassay utilizing monoclonal antibodies reactive to two non-interfering polypeptide epitopes is generally used, but a competitive binding assay may also be employed.

Various methods such as Scatchard analysis in conjunction with radioimmunoassay techniques may be used to assess the affinity of antibodies a target polypeptide. Affinity is expressed as an association constant, Kₐ, which is defined as the molar concentration of target polypeptide-antibody complex divided by the molar concentrations of free antigen and free antibody under equilibrium conditions. The Kₐ is determined for a preparation of polyclonal antibodies, which are heterogeneous in their affinities for multiple target polypeptide epitopes, represents the average affinity, or avidity, of the antibodies for target polypeptides. The Kₐ is determined for a preparation of monoclonal antibodies, which are monospecific for a particular target polypeptide epitope, represents a true measure of affinity. High-affinity antibody preparations with Kₐ ranging from about 10⁹ to 10¹² L/mole are preferred for use in immunoassays in which the target polypeptide-antibody complex must withstand rigorous manipulations. Low-affinity antibody preparations with Kₐ ranging from about 10⁶ to 10⁷ L/mole are preferred for use in immunopurification and similar procedures that ultimately require dissociation of target polypeptides, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparation for certain downstream applications. For example, a polyclonal antibody preparation containing at least 1-2 mg specific antibody/ml, preferably 5-10 mg specific antibody/ml, is generally employed in procedures requiring precipitation of target polypeptide-antibody complexes. Procedures for evaluating antibody specificity, titer, and avidity, and guidelines for antibody quality and usage in various applications, are generally available. (See, e.g., Catty, supra, and Coligan et al. supra.)

In an alternative embodiment, polypeptide involved in the BMP and Bmpr1a pathways may be mutagenized. This can be accomplished by forming a vector having a promoter and a PTEN pathway gene. The PTEN pathway genes include Noggin, PTEN, Akt, GSK-3, cyclin D1, Tert (Telomerase reverse transcriptase), PI3K, SMAD1,5,8, p27, and mutant genes related thereto. PTEN pathway component effects occur downstream from the BMP-Bmpr1a receptor triggering event taking place at the hair follicle cell membrane. By activating these PTEN pathway genes, effects similar to the mutagenesis of the Bmpr1a gene can be achieved, since both routes lead to the diminution of effects of BMP signaling. The PTEN pathway vector can be utilized in vitro or in vivo. Preferably, the PTEN pathway vector can be used to induce hair follicle cell proliferation, differentiation, or apoptosis. As previously described, these can be conditional or conventional mutants. Also, eukaryotic cells, tissue, or organisms can be influenced by the PTEN pathway vector.

Prokaryotic organisms, such as bacterial species, containing a prokaryotic vector can also be developed. The prokaryote will include a Wt or mutant PTEN pathway nucleotide sequence. PTEN pathway components can be produced in large quantities and purified from cultured prokaryotic organisms.

Hair Follicle Stem Cell Cultivation Systems

Yet another aspect of the invention encompasses a hair follicle stem cell cultivation system. For example, an in vitro hair follicle stem cell cultivation system may be developed, wherein an activated hair follicle stem cell population proliferates. The cultivation system includes an isolated hair follicle tissue, a culture medium, and an isolated stem cell activator. The activator operatively attaches to at least one stem cell in the population. The activator can be a mutant Bmpr1a receptor polypeptide, a mutant Bmpr1a receptor nucleic acid sequence, anti-Bmpr1a antibody, anti-BMP antibody, a Wt Bmpr1a receptor antisense sequence, Noggin polypeptide, a BMP polypeptide, or a fragment thereof. The hair follicle tissue can be of mammalian origin. In particular, human HF tissue can be isolated, containing cells that are then mutagenized to prevent BMP and Bmpr1a interaction. Inhibition of BMP should cause HFSC proliferation. It can also result in tumor formation in vitro. Additionally, the HFSCs can be studied. Preferably, the HFSC is in an isolated hair follicle derived from a host organism.

An exemplary in vitro hair follicle tissue cultivation system causes HFSC population proliferation in response to a Noggin activator. Other activators, such as anti-BMP and anti-Bmpr1a anti-Bmpr1a antibodies, anti-BMP antibodies, or fragments thereof may be used. This cultivation system contains isolated hair follicle tissue, culture medium, and an effective amount of isolated Noggin polypeptides, or other activators. Plucked hair follicle tissue may be used. Alternatively, instead of tissue, the cultivation system can contain an isolated hair follicle stem cell population comprising at least 10⁴ cells. The hair follicle stem cell population can be isolated by FACS methods using antibodies directed against HFSC-associated antigens, such as anti-Bmpr1a receptor polypeptide. Isolated Noggin polypeptides, which include truncated polypeptides or Noggin fragments, are contacted in vitro with the Bmpr1a cell receptors. Bmpr1a receptor binding to BMP is substantially inhibited by Noggin.

The activator can be placed in operative contact with the hair follicle stem cell population by means of an activator insertion device. Activator insertion devices can be injection, diffusion, particle-mediated, micro-vessel encapsulation, or liposome encapsulation devices. An in vitro mutant Bmpr1a hair follicle stem cell cultivation system results, wherein a mutant hair follicle stem cell population proliferates, having the following: an isolated mutant Bmpr1a hair follicle stem cell population comprising an inactive Bmpr1a receptor and culture medium. Bmpr1a gene mutations introduced into the mutant hair follicle stem cell can be frame shift, substitution, loss of function, or deletion mutations.

In an alternative in vitro cultivation system, hair follicle tissue containing mutant Bmpr1a HFSCs is isolated and cultivated with culture medium in a culture vessel. The isolated mutant Bmpr1a HFSCs contain inactive Bmpr1a receptor nucleic acids and polypeptides. The HFSCs may also lack Bmpr1a receptor nucleic acids and polypeptides. The isolated mutant Bmpr1a HFSC receptor polypeptides may exist either intracellularly, extracellularly, in cytosol, or on the cell surface.

In yet another embodiment, a tissue system can be developed utilizing an isolated hair follicle tissue sample, where this sample is cultivated in a culture medium. The tissue is isolated from the epidermis and will include HFSCs. Plucked hair follicles may be used as a tissue source. Vectors, previously discussed, can be used to transfect the cells. The tissue cells will be allowed to proliferate, with the phenotypic results of the mutants then observed. As such, transfected HFSCs containing deleted Exon 2 of Bmpr1a genes, deleted Bmpr1a genes, and inactive Bmpr1a gene derivatives thereof are also part of the present invention. These transfected HFSCs include Bmpr1a mutant HFSCs expressing inactive Bmpr1a polypeptides. Bmpr1a mutant organisms containing the transfected HFSCs or Bmpr1a mutant HFSCs are part of the invention.

Animal Models

In an additional aspect, any of the mutant organisms identified herein may be utilized to study human disorders. By way of example, the aforementioned mouse model can be used for studying human matricomas. Inactivation of the Bmpr1a receptor causes formation of tumor cells throughout the hair follicle tract. Hair follicle cells studied are a hair bulb (HB), dermal papilla (DP), dermal sheath (DS), precortex (PC), inner root sheath (IRS), outer root sheath (ORS), hair shaft (HS), bulge (Bu), arrector pili muscle (APM), and sebaceous gland (SG) using previously described cell markers.

Kits

A variety of kits can be formed either from the mutant or Wt polypeptides or the nucleic acid sequences associated with hair follicle tissue or cells. Kits are described for detection of mutant or variant forms of the aforementioned nucleic acid molecules, detection of expressed polypeptides or proteins, and measurement of corresponding levels of protein expression. Kits can detect the presence or absence of mutants or Wt forms of the nucleic acid molecules, and their expressed amino acid sequences or polypeptide molecules. The kit will preferably have a container and either a nucleic acid molecule or a polypeptide molecule, which includes any of the aforementioned sequences.

A kit will be formed with a container and a Bmpr1a polypeptide molecule. The kit will detect either a mutant or Wt Bmpr1a polypeptide or nucleic acid molecule in hair follicle tissue or cells. Specifically, the kit will be used to detect the presence of a mutant Bmpr1a receptor, gene, or polypeptide. The kit will also detect a mutant HFSC containing an inactive Bmpr1a receptor or gene. Kits for detection and quantitation of the presence in hair follicle cells of markers such as Bmpr1a, BMP, Noggin, PTEN, P-PTEN, AKT, P-AKT, Tert, -catenin, Ki67, p27, Smad1,5,8, tubulin, Chromgrin A, BAD, P-BAD, and FAK polypeptide and nucleic acid markers will be formed. These kits can be used for detection and quantitation of markers associated with hair follicle cell activation, proliferation, differentiation, apoptosis, polyposis, and tumor formation. Specifically, immunodiagnostics and nucleic acid probe kits for mutant Bmpr1a hair follicle cell expression of the foregoing marker nucleic acid sequence and polypeptide markers will be made and used. In addition, the present invention includes diagnostic methods and kits for the prediction and assessment of hair follicle tumorigenesis. These foregoing kits may be used either in vitro or in vivo.

In summary, hybridization methodology and kits for the detection, identification, and quantitation of Bmpr1a-associated nucleic acid sequences in cells are set forth herein. Using these methods, Bmpr1a Wt and mutant nucleic acid sequences can be identified, characterized, and quantified. In addition, kits may be produced utilizing Bmpr1a-derived nucleic acid molecule standards, antibodies, and kit components as previously described.

All publications, patents, patent applications, and other references cited in this application are herein incorporated by reference in their entirety as if each individual publication, patent, patent application or other reference were specifically and individually indicated to be incorporated by reference.

### DEFINITIONS

The following definitions define terms used herein:

Activated mutant is a post-recombination organism, tissue, or cell wherein the mutant is obtained by injection of a recombination activator into a conditional mutant organism, tissue, or cell to induce a mutation event that results in inactivation of the targeted gene. For example, an activated Bmpr1a mutant organism is a post-excision organism that resulted from PolyI:C injection of a conditional Bmpr1a mutant organism to yield a nonfunctional Bmpr1a gene.

Activator is a molecule that activates a cellular activity. Cellular activities induced by the activator may be proliferation, self-renewal, differentiation, tumorigenesis, or apoptosis. A hair follicle stem cell activator is one that generally activates proliferation, self-renewal, or differentiation. The HY activator may optionally induce tumorigenesis or apoptosis in a cell. A hair follicle stem cell activator generally induces proliferation or cell division. Activator can also refer to a molecule that induces recombination in a cell, such as those utilized in the Lox and Flp recombinase genetic systems. Examples of recombination activators are PolyI:C and interferon, which induce recombination in cells containing Lox or Frt flanked genes, generally resulting in inactivation of the target gene.

Allele is a shorthand form for allelomorph, which is one of a series of possible alternative forms for a given gene differing in the DNA sequence and affecting the functioning of a single product.

An amino acid (aminocarboxylic acid) is a component of proteins and peptides. All amino acids contain a central carbon atom to which an amino group, a carboxyl group, and a hydrogen atom are attached. Joining together of amino acids forms polypeptides. Polypeptides are molecules containing up to 1000 amino acids. Proteins are polypeptide polymers containing 50 or more amino acids.

Anagen is an active growth phase of hair follicles. During anagen, the cells in the root of the hair divide rapidly, adding to the hair shaft. In humans, hair growth during this phase is about 1 cm every 28 days. Scalp hair stays in the anagen phase of growth for 2-6 years. Hairs may be plucked in anagen phase for growth in vitro. Plucked hair follicles may be cultivated in vitro.

Antibody (Ab) is any molecule that can bind specifically to an Antigen (Ag). Each Ab molecule has a unique Ag binding site that enables it to bind specifically to its corresponding antigen. Abs include, but are not limited to, immunoglobulins of the IgG, IgA, IgM, IgD, and IgE classes. Abs are often produced by B cells and plasma cells in response to infection or immunization, bind to and neutralize pathogenic micro-organisms, or prepare microbes for uptake and destruction by phagocytes. Abs may also be produced in vitro by cultivation of plasma cells or B cells, or by utilization of genetic engineering technologies.

An antigen (Ag) is any molecule that can bind specifically to an antibody (Ab). Ags can stimulate the formation of Abs. Each Ab molecule has a unique Ag binding pocket that enables it to bind specifically to its corresponding antigen. Abs may be used in conjunction with labels (e.g., enzyme, fluorescence, radioactive) in histological analysis of the presence and distribution of marker Ags. Abs may also be used to purify or separate cell populations bearing marker Ags through methods, including fluorescence activated cell sorter (FACS) technologies. Abs that bind to cell surface receptor Ags can inhibit receptor-specific binding to other molecules to influence cellular function. Abs are often produced in vivo by B cells and plasma cells in response to infection or immunization, bind to and neutralize pathogens, or prepare them for uptake and destruction by phagocytes. Abs may also be produced in vitro by cultivation of plasma cells, B cells or by utilization of genetic engineering technologies.

Bone morphogenic proteins (BMPs) constitute a novel subfamily of the transforming growth factor type beta (TGF-beta) supergene family and play a critical role in modulating mesenchymal differentiation and inducing the processes of cartilage and bone formation. BMPs induce ectopic bone formation and support development of the viscera. Exemplary BMPs include those listed by the NcBI, such as human BMP-3 (osteogenic) precursor (NP001192), mouse BMP-6 (NP031582), mouse BMP-4 (I49541), mouse BMP-2 precursor (1345611), human BMP-5 preprotein (NP 066551.1), mouse BMP-6 precursor (1705488), human BMP-6 (NP 001709), mouse BMP-2A (A34201), mouse BMP-4 (461633), and human BMP-7 precursor (4502427). The BMPs consist of at least eight members, BMP-2 through BMP-8A and BMP-8B. In embryogenesis, BMPs play roles in dorsoventral and/or anterior-posterior axis formation. Bone morphogenic protein (BMP) initiates, promotes, and regulates bone development, growth, remodeling, and repair. BMPs belong to the TGF-beta superfamily of structurally related signaling proteins. Members of this TGF-beta superfamily are widely represented throughout the animal kingdom and have been implicated in a variety of developmental processes. Proteins of the superfamily are disulfide-linked dimers composed of two 12-15 kDa polypeptide chains.

Bmpr1a receptor, or Bmpr1a, is defined as the bone morphogenetic protein receptor, type 1A, expressed almost exclusively in skelet al muscle. Bmpr1a is a regulator of chondrocyte differentiation, down stream mediator of Indian Hedgehog, TGFBR superfamily, and activin receptor-like kinase 3. Ligand binding to receptor induces the formation of a complex in which the Type II BMP receptor phosphorylates and activates the Type I BMP receptor. The Type I BMP receptor then propagates the signal by phosphorylating a family of signal transducers, the Smad proteins. The Bmpr1a gene encodes the Bmpr1a receptor. Bone morphogenic protein receptor, type A receptor (Bmpr1a receptor) is encoded by the Bmpr1a gene. Bmpr1a binds to BMP and Noggin. Bmpr1a is known to be a regulator of chondrocyte differentiation, and downstream mediator of Indian Hedgehog, TGFBR superfamily, and activin receptor-like kinase 3. Ligand binding to receptor induces the formation of a complex in which the Type II BMP receptor (Bmpr1b receptor) phosphorylates and activates the Type I BMP receptor (Bmpr1a receptor). The Type I BMP receptor (Bmpr1a) then propagates the signal by phosphorylating a family of signal transducers, the Smad proteins.

Bmpr1a mutant organism is defined as an organism lacking a functional Bmpr1a gene or a conditionally activated Bmpr1a gene that can be rendered nonfunctional, where a nonfunctional Bmpr1a gene is one that encodes an inactive Bmpr1a receptor. An example of such an organism is the Mx1-Cre⁺Bmpr1a^{fx/fx} mutant mouse.

Bmpr1a gene (Bone morphogenetic protein receptor, type 1A gene)—is any Bmpr1a gene isolated from an organism, including human and mouse, as represented in SEQ ID NOs. 8 and 1 respectively.

Catagen is a short transition phase that occurs at the end of anagen. It signals the end of the active growth of a hair. This phase, in humans, lasts for about 2-3 weeks while a club hair is formed.

Chimera is an individual composed of a mixture of genetically different cells. By definition, genetically different cells of chimeras are derived from genetically different zygotes.

Club hair is a hair in resting stage prior to shedding, in which the bulb has become a club-shaped mass.

Conditional Bmpr1a mutant knockout organism can be a pre-recombination or post-recombination Bmpr1a mutant organism. An example of a conditional Bmpr1a mutant knockout organism is a Mx1-Cre⁺Bmpr1a^{fx/fx} or Mx1-Cre⁺Bmpr1a^{fx/fx} Z/EG organism. The mutant organism may be a mouse. Upon administration of a recombination activator, such as PolyI:C, to the pre-recombination Bmpr1a mutant organism, a post-recombination Bmpr1a mutant organism is formed in which the cells may contain a mutant Bmpr1a nucleic acid sequence. The recombination activator may be administered either prenatally or postnatally to induce Bmpr1a mutation in the cells.

Conditional mutant is a pre-recombination organism, tissue, or cell wherein injection of a recombination activator into the conditional mutant organism, tissue, or cell induces a mutation event that results in inactivation of the targeted gene, resulting in formation of an activated Bmpr1a mutant organism.

Deletion mutations may be conditional knockout deletion mutations or conventional deletion mutations. Knockout deletion mutations are induced by administration of a recombination activator, such as PolyI:C, to a pre-excision mutant organism. Injection of the recombination activator results in excision or “knockout” of a portion of the genetic sequence from the nucleic acid sequence, thereby inducing a deletion mutation in the gene. Conventional deletion mutations may be single or multiple nucleotide deletions in a gene or chromosome.

Differentiation occurs when a cell transforms itself into another form. For example, a hematopoietic stem cell (HSC) may differentiate into cells of the lymphoid or myeloid pathways. The HSC might differentiate into lymphocytes, monocytes, polymorphonuclear leukocytes, neutrophils, basophils, or eosinophils. Similarly, a hair follicle stem cell (HFSC) may differentiate into cells of the hair bulb and hair shaft.

Expression cassette (or DNA cassette) is a deoxyribonucleic acid (DNA) sequence that can be inserted into a cell's DNA sequence. The cell in which the expression cassette is inserted can be a prokaryotic or eukaryotic cell. The prokaryotic cell may be a bacterial cell. The expression cassette may include one or more markers, such as Neo and/or LacZ. The cassette may contain stop codons. In particular, a Neo-LacZ cassette is an expression cassette that can be placed in a bacterial artificial chromosome (BAC) for insertion into a cell's DNA sequence. Such expression cassettes can be used in homologous recombination to insert specific DNA sequences into targeted areas in known genes.

A gene is a hereditary unit that has one or more specific effects upon the phenotype of the organism; and the gene can mutate to various allelic forms. The gene is generally comprised of deoxyribonucleic acid or ribonucleic acid sequences (DNA, RNA).

Green fluorescent protein (GFP) is a spontaneously fluorescent protein isolated from coelenterates, such as the Pacific jellyfish, Aequoria victoria. It transduces, by energy transfer, the blue chemiluminescence of another protein, aequorin, into green fluorescent light. GFP can function as a protein tag to a broad variety of proteins, many of which have been shown to retain native function upon GFP binding. GFP is used as a noninvasive marker in living cells to allow numerous other applications such as a cell lineage tracer, reporter of gene expression and as a potential measure of protein-protein interactions. Green fluorescent protein is comprised of 238 amino acids.

A host cell is a cell that receives a foreign biological molecule, including a genetic construct or antibody, such as a vector containing a gene.

A host organism is an organism that receives a foreign biological molecule, including a genetic construct or antibody, such as a vector containing a gene.

HFSC is a hair follicle stem cell that plays a critical role in hair growth and epidermis maintenance. HFSCs are located in the bulge region and the secondary hair germ.

Hair follicle is comprised of a cycling segment and a permanent segment. The cycling segment includes a hair bulb (HB), precortex (PC), inner root sheath (IRS), outer root sheath (ORS), and hair shaft (HS). The hair bulb (HB) includes the hair matrix (HM), dermal papilla (DP), and dermal sheath (DS). The permanent segment includes the bulge (Bu), arrector pili muscle (APM), sebaceous gland (SG), and hair shaft. The hair shaft protrudes externally through the epidermis.

The wild type (Wt) hair follicle stem cell (HFSC) forms the hair shaft and inner root sheath (IRS) regions, along with the outer root sheath (ORS) region of the hair. The bulge-located HFSCs form the outer root sheath, the inner root sheath and hair shaft (HS). Thus, the wild type HFSC generates the entire structure of a hair, which includes IRS, ORS, HS, bulge (Bu), arrector pili muscle (APM), and sebaceous gland (SG). Aberrant HFSC function occurs when a Bmpr1a mutation is introduced into the HFSC. Mutant Bmpr1a HFSCs self-renew and proliferate, generating a HF structure; however, matricomass can also result from such mutant HFSCs.

Hair follicle tissue is isolated large or small intestine tissue obtained from an organism, and this tissue possesses IRS, ORS, HS, bulge, APM, SG elements, and other hair follicle microstructures, or portions thereof. Hair follicle tissue can be derived from either Wt or mutant organisms. Hair follicle tissue includes hair follicle stem cells. Hair follicle tissue may be cultivated in vitro or in vivo.

Host cell is a cell that receives a foreign biological molecule, including a genetic construct or antibody, such as a vector containing a gene.

Host organism is an organism that receives a foreign biological molecule, including a genetic construct or antibody, such as a vector containing a gene.

Insertion device is a device that places an activator or oligonucleotide molecule in operative contact with a cell. Insertion devices can be activator insertion devices or oligonucleotide insertion devices. The activator or oligonucleotide molecule may be inserted within the cell or placed in contact with the cell surface. The insertion device can be injection, electroporation, transfection, vector, particle encapsulation, or liposome encapsulation devices.

Knockout is an informal term coined for the generation of a mutant organism (generally a mouse) containing a null or inactive allele of a gene under study. Usually the animal is genetically engineered with specified wild-type alleles replaced with mutated ones. Knockout also refers to the mutant organism or animal. The knockout process may involve administration of a recombination activator that excises a gene, or portion thereof, to inactivate or “knock out” the gene. The knockout organism containing the excised gene produces a nonfunctional polypeptide.

Label is a molecule that is used to detect or quantitate a marker associated with a cell or cell type. Labels may be nonisotopic or isotopic. Representative, nonlimiting nonisotopic labels may be fluorescent, enzymatic, luminescent, chemiluminescent, or colorimetric. Exemplary isotopic labels may be H³, C¹⁴, or P³². Enzyme labels may be horseradish peroxidase, alkaline phosphatase, or β-galactosidase labels conjugated to anti-marker antibodies. Such enzyme-antibody labels may be used to visualize markers associated with cells in hair follicle or other tissue.

Marker is an indicator that characterizes either a cell type or a cell that exists in a particular state or stage. A stem cell marker is a marker that characterizes a specific cell type that can possess a cell function such as self-renewal, proliferation, differentiation, or apoptosis. The marker may be external or internal to the cell. An external marker may be a cell surface marker. An internal marker may exist in the nucleus or cytoplasm of the cell. Markers can include, but are not limited to polypeptides or nucleic acids derived from Bmpr1a, BMP, Noggin, PTEN, PPTEN, AKT, PAKT, Tert, β-catenin, Ki67, p27, Smad1,5,8, tubulin, Chromgrin A, BAD, PBAD, FAK, GFP, and LacZ molecules, and mutant molecules thereof. Markers may also be antibodies to the foregoing molecules, and mutants thereof. For example, antibodies to Bmpr1a, BMP, and Noggin can serve as markers that indicate the presence of these respective molecules within cells, on the surface of cells, or otherwise associated with cells. GFP and LacZ marker sites can indicate that recombination occurs in a target gene, such as the Bmpr1a gene.

Mutation is defined as a genotypic or phenotypic variant resulting from a changed or new gene in comparison with the Wt gene. The genotypic mutation may be a frame shift, substitution, loss of function, or deletion mutation, which distinguishes the mutant gene sequence from the Wt gene sequence.

Mutant is an organism bearing a mutant gene that expresses itself in the phenotype of the organism. Mutants may possess either a gene mutation that is a change in a nucleic acid sequence in comparison to Wt, or a gene mutation that results from the elimination or excision of a sequence. In addition polypeptides can be expressed from the mutants. Mutant may also refer to nucleic acid or polypeptide sequences themselves that result from gene mutation.

Noggin is a polypeptide that is an inhibitor of bone morphogenic proteins (BMPs), and its inhibitory activity is manifested through binding to Bmpr1a receptor. Noggin binding to Bmpr1a receptor induces HFSC proliferation and self-renewal functions. Noggin's role in BMP signaling is required for embryonic growth and patterning of the neural tube and somite. Noggin is also essential for cartilage morphogenesis and joint formation. Mouse Noggin polypeptide and nucleic acid sequences are SEQ ID NOs. 11 and 12 respectively. Human polypeptides and nucleic acid sequences are SEQ ID NOs. 9 and 10 respectively.

Nucleic acid or nucleotide sequence is a nucleotide polymer. Nucleic acid also refers to the monomeric units from which DNA or RNA polymers are constructed, wherein the unit consists of a purine or pyrimidine base, a pentose, and a phosphoric acid group.

Nucleotide sequence is a nucleotide polymer, including genes, gene fragments, oligonucleotides, polynucleotides, and other nucleic acid sequences.

Plasmids are double-stranded, closed DNA molecules ranging in size from 1 to 200 kilo-bases. Plasmids are capable of extrachromosomal replication, like other episomes; and some plasmids are capable of integrating into the host genome. Plasmids may be contained in cloning vectors for transfecting a host with a nucleic acid molecule.

PolyI:C is an interferon inducer consisting of a synthetic, mismatched double-stranded RNA. The polymer is made of one strand each of polyinosinic acid and polycytidylic acid. PolyI:C is 5′-Inosinic acid homopolymer complexed with 5′-cytidylic acid homopolymer (1:1). PolyI:C's pharmacological action includes antiviral activity.

Polypeptide is an amino acid polymer comprising at least two amino acids.

Post-excision mutant organism is an organism, a targeted gene, or sections thereof, wherein the targeted gene or section has been excised by recombination. The post-excision organism is called a “knockout” organism. Administration of a recombination activator, such as PolyI:C or interferon, can induce the recombination event resulting in target gene excision. A post-excision Bmpr1a mutant organism is one in which the Bmpr1a gene has been inactivated.

Pre-excision Bmpr1a mutant organism is one that has recombination sites flanking regions of the Bmpr1a gene. The pre-excision organism generally has recombinase-encoded sites that can be induced to express Cre or Flp recombinase, but remain dormant or unexpressed until cells of the organism are exposed to a recombination activator. Administration of the activator to the pre-excision Bmpr1a mutant organism under proper conditions can transform it into a post-excision Bmpr1a mutant organism.

Post-excision Bmpr1a mutant organism is one which has been administered a recombination activator, such as PolyI:C, which excises a recombination site-flanked portion of the Bmpr1a gene, rendering it nonfunctional. The mutant Bmpr1a organism contains cells that possess inactive Bmpr1a polypeptides, whose binding to BMP has been substantially inhibited.

Proliferation occurs when a cell divides and results in progeny cells. Proliferation can occur in the self-renewal or proliferation zones of the hair follicle villus. Stem cells may undergo proliferation upon receipt of molecular signals such as those transmitted through Bmpr1a cellular receptor.

PTEN family nucleotide sequence includes, but is not limited to, the following: PTEN, PI3K, Akt, Tert, β-catenin, Smad 1,5,8, P27, and BAD nucleic acid sequences, and mutant sequences derived therefrom.

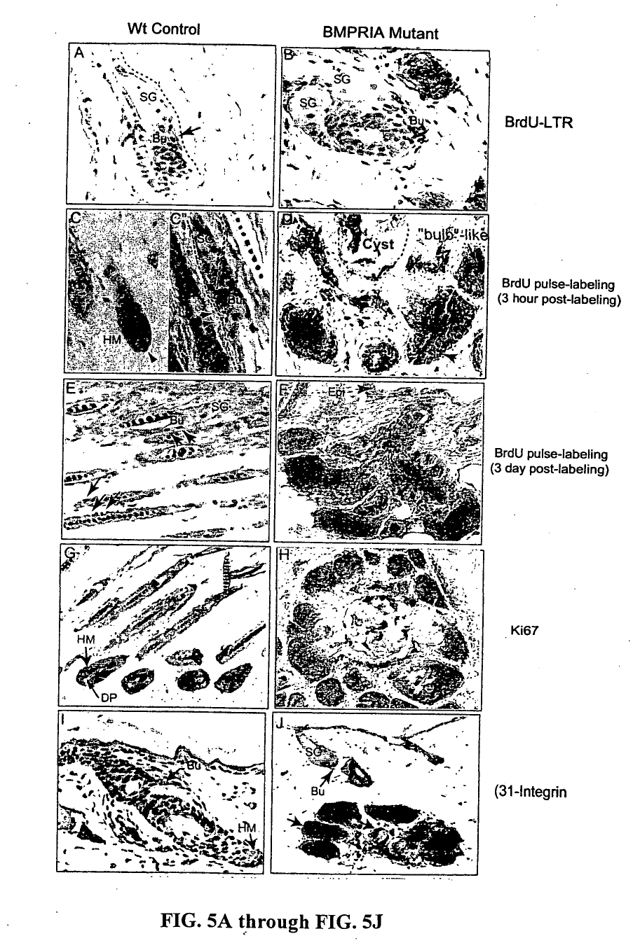

PTEN pathway polypeptides or proteins are those that are encoded by PTEN pathway genes, which include, but are not limited to the following: PTEN, PI3K, Akt, Tert, β-catenin, Smad 1,5,8, P27, and BAD genes, and mutant genes derived therefrom. The PTEN pathway, also called the PTEN/PI3K/Akt/Tert/β-catenin pathway, is depicted diagrammatically in FIG. 5B. The PTEN pathway is regulated by Noggin and BMP, which function in a diametrically opposite manner. Noggin binding to Bmpr1a receptor releases BMP inhibition of ISC function, through a cascade of increased levels of activated P-PTEN, P-Akt, β-catenin, and Tert, resulting in ISC proliferation necessary to regenerate dead or lost hair follicle epithelial cells in the intestine. In contrast, high BMP activity at the tips of the villi induces increased BAD activity and hair follicle cell death; whereas Bmpr1a mutant villi, nonresponsive to BMP signaling, exhibited decreased apoptosis due to loss of BAD signaling.

A regulator is molecule that regulates an activity of a cell. Regulators include, but are not limited to, BMP, Noggin, or Ly294002. A regulator may cause increase or decrease in an activity of a cell or cell population such as proliferation, self-renewal, differentiation, polyposis, or tumorigenesis. An activator is a regulator that causes an increase in activity. An inhibitor is a regulator that causes a decrease in activity or prevents the occurrence of an activity.

A selectable marker is a marker that is inserted in a nucleic acid sequence that permits the selection and/or identification of a target nucleic acid sequence or gene. A selectable marker associated with Bmpr1a gene mutation may identify the presence of the Bmpr1a mutation.

Self-renewal occurs where a cell reproduces an exact replicate of itself, such that the replicate is identical to the original stem cell.

Smad proteins are signal transducers that interact with BMP receptors. SMAD molecules are evolutionarily conserved proteins identified as mediators of transcriptional activation by members of the TGF-beta superfamily of cytokines, including TGF-beta, Activins, and BMP. Upon activation these proteins directly translocate to the nucleus where they may activate transcription (Datto et al.). Eight Smad proteins have been cloned (Smad 1-7 and Smad 9). Upon phosphorylation by the BMP Type I receptor, Smad1 can interact with either Smad4 or Smad6. The Smad1-Smad6 complex is inactive; however, the Smad1-Smad4 complex triggers the expression of BMP responsive genes. The ratio between Smad4 and Smad6 in the cell can modulate the strength of the signal transduced by BMP. Smad1,5,8 is also referred to as SMAD158. SMAD-1 is the human homologue of Drosophila Mad (Mad=Mothers against decapentaplegic). SMAD-1 has been shown to migrate into the nucleus in response to BMP-4. SMAD-5 sequences have been cloned. An analysis of various tumors demonstrates that mutations in various SMAD genes do not, in general, account for the widespread resistance to TGF-beta that is found in human tumors. SMAD-8 is a protein from Xenopus laevis distantly related to other SMAD proteins, and it modulates the activity of BMP-4.

Small molecules are defined as regulatory polypeptide or nucleic acid molecules that cause detectable changes in protein-protein interaction systems that may also affect one or more phenotypic changes. These small molecules may operatively function by structural similarity to and competitive inhibition with native molecules in vitro or in vivo. Phenotypic changes may include observed changes in such parameters as hair loss, hair growth, and matricomas development. Small regulatory polypeptide molecules include, but are not limited to, antibody fragments such as Fab, F(ab)₂, Fv, and antibody combining regions. Small regulatory nucleic acid molecules include, but are not limited to, antisense RNA sequences that interfere with wild type polypeptide function; and truncated nucleic acid sequences that encode shortened polypeptides that interfere with function.

A stem cell is defined as a pluripotent or multipotent cell that has the ability to divide (self-replicate) or differentiate for indefinite periods—often throughout the life of the organism. Stem cell self-renewal occurs when a stem cell divides to create an identical cloned replicate of itself. Under the right conditions, or given optimal regulatory signals, stem cells also can differentiate to transform themselves into the many different cell types that make up the organism. Stem cells may be distinguishable from progeny daughter cells by such traits as BrdU retention and physical location/orientation in the hair follicle microenvironment. Multipotential or pluripotential stem cells possess the ability to differentiate into mature cells that have characteristic attributes and specialized functions, such as hair follicle cells, epidermis cells, intestinal cells, blood cells, cardiac cells, or nerve cells.

Stem cell marker is defined as a specialized protein or glycoprotein, associated with the stem cell, that is characteristic of that cell type. Stem cell markers often exist on the surface of cells, and these markers, sometime referred to as “receptors,” may have the functional capability of selectively binding or adhering to a “signaling” molecule. There are many different types of receptors that differ in their structure and affinity for the signaling molecules. For example, the Bmpr1a receptor marker on HFSCs may bind to either BMP or Noggin signaling molecules. Typically, the stem cell marker is antigenic and can be characterized as an antigen capable of binding to specific ant-marker antibodies. More particularly, the stem cell marker may characterize the stem cell as a multipotential or pluripotential cell type rather than as a differentiated cell type.

A stem cell population is a population that possesses at least one stem cell.

Support is defined as establishing viability, growth, proliferation, self-renewal, maturation, differentiation, and combinations thereof, in a cell. In particular, to support a HFSC population refers to promoting viability, growth, proliferation, self-renewal, maturation, differentiation, and a combination thereof, in the HFSC population. Support of a cell may occur in vivo or in vitro.

Telogen is the resting phase of the hair follicle. At any time point, about 10% to 15% of all hairs are in telogen phase. In humans, this phase lasts about 100 days for hairs on the scalp, and much longer for hairs on the eyebrow, eyelash, arm and leg. During this phase, the hair follicle is completely at rest and the club hair is completely formed. About 25-100 telogen hairs are shed in humans normally each day.

A truncated Bmpr1a sequence is shortened with respective to the Wt Bmpr1a sequence. For example, a truncated Bmpr1a nucleic acid sequence may be a sequence in which Exon 2 has been excised. A truncated Bmpr1a nucleic acid sequence having an excised Exon 2 region encodes an inactive Bmpr1a polypeptide, whose binding to BMP has been substantially inhibited. Other truncated Bmpr1a nucleic acid sequences may encode inactive Bmpr1a polypeptide molecules. The truncated Bmpr1a nucleic acid sequence includes a sequence comprising zero (0) nucleotides, where the Bmpr1a gene has been entirely deleted.

A vector is an autonomously self-replicating nucleic acid molecule that transfers a target nucleic acid sequence into a host cell. The vector's target nucleic acid sequence can be a Wt or mutant gene, or fragment derived therefrom. The vector can include a gene expression cassette, plasmid, episome, or fragment thereof. Gene expression cassettes are nucleic acid sequences with one or more targeted genes that can be injected or otherwise inserted into host cells for expression of the encoded polypeptides. Episomes and plasmids are circular, extrachromosomal nucleic acid molecules, distinct from the host cell genome, which are capable of autonomous replication. The vector may contain a promoter, marker or regulatory sequence that supports transcription and translation of the selected target gene. Viruses are vectors that utilize the host cell machinery for polypeptide expression and viral replication.

Wild type is the most frequently observed phenotype or genotype in a population, or the one arbitrarily designated as “normal.” Wild type is often represented by “+” or “Wt” symbols. The Wt phenotype is distinguishable from mutant phenotypic variations, and the Wt genotype is distinguishable from mutant genotypic variations.

As various changes could be made in the above compositions, methods, and products without departing from the scope of the invention, it is intended that all matter contained in the above description and in the examples given below, shall be interpreted as illustrative and not in a limiting sense.

### EXAMPLES

The following examples illustrate the invention.

### Example 1

An inducible pre-excision Bmpr1a knockout mouse was generated to enable characterization of the effect of Bmpr1a gene deletion upon hair follicle stem cell (HFSC) proliferation, differentiation, and tumorigenesis. The conditional knockout Bmpr1a mouse was obtained by crossing a Bmpr1a^{fx/fx} mouse line with an interferon-inducible Mx1-Cre mouse line. A heterozygous Bmpr1a^{±} mouse was also used to generate a Bmpr1a^{fx/-} mouse as a control.

The Bmpr1a^{fx/fx} mouse line was obtained by targeting vector-mediated insertion of LoxP sites into the Bmpr1a locus of mouse embryonic stem (ES) cells. One LoxP site was placed in intron 1 of the Bmpr1a gene, and two other flanking LoxP sites were located in an EcoRI site in intron 2 surrounding a PGK-neo expression cassette. The PGK-neo expression cassette introduced Bg/I and EcoRV restriction sites into the wild type Bmpr1a gene, and the cassette was inserted in reverse orientation relative to the direction of Bmpr1 transcription between the two Bmpr1a intron regions. The first site was marked by an NheI site for Southern blot analysis. Linearized targeting vectors with the expression cassette were electroporated into the ES cells that were subsequently cultured in the presence of G418 and FIAU on inactivated STO fibroblasts. Transfected clone 35H3 was characterized by the presence of both a wild type allele (+) and a targeted allele termed the floxP+neo (fn) allele. Subsequent Cre-dependent recombination yielded three alleles: floxP (fx), Δexon2+neo (Δe2n), and Δexon2 (Δe2). ES clones containing these alleles were distinguishable on Southern blot analysis with NheI and SacI marker sites.

Transfected LoxP site-containing ES cells were cloned and grown in the presence of irradiated fibroblast cells. The ES cell clone 35H3 was microinjected into C57BL/6J blastocysts for germ line transmission and implantation into the uterine horns of day 2.5 pseudopregnant foster mothers. Chimeras were identified among progeny mice by the presence of agouti fur, and these progeny were bred with C57BL/6 mice to obtain mutant homozygous Bmpr1a^{fx/fx} mice.

Mutant Bmpr1a^{fx/fx} mice were crossed with Mx1-Cre mice (Jackson Laboratory, Bar Harbor, Me., #3556, #2527), yielding litters containing pups with homozygous Mx1-Cre⁺Bmpr1a^{fx/fx} (Bmpr1a mutant), heterozygous Mx1-Cre⁺Bmpr1a^{fx/+}, wild type control Mx1-Cre⁻Bmpr1a^{fx/fx}, and wild type control Mx1-Cre⁻Bmpr1a^{fx/+} genotypes. The resultant Bmpr1a^{fx/fx} mouse line contained a second Exon of the Bmpr1a gene that was flanked by two LoxP sites. This pre-excision Mx1-Cre⁺Bmpr1a^{fx/fx} conditional mutant mouse permitted subsequent recombination activator-induced excision of LoxP-flanked Exon 2 of the Bmpr1a gene, resulting in expression of an inactive Bmpr1a receptor polypeptide in the post-excision Bmpr1a mutant mouse.

### Example 2

The pre-excision Mx1-Cre⁺Bmpr1a^{fx/fx} mutant mouse obtained in the previous example was injected postnatally with PolyI:C, a recombination activator, to induce excision of Exon2 of the Bmpr1a gene. The Bmpr1a locus was successfully targeted for excision by three injections of the PolyI:C recombination activator at two-day intervals, as demonstrated by PCR results obtained three months after birth. Thus, it was determined that a post-excision Mx1-Cre⁺Bmpr1a^{fx/fx} mutant mouse resulted that possessed mutant Bmpr1a genes encoding inactive and truncated Bmpr1a receptor polypeptide.

Mx1-Cre Bmpr1a mutant pups were injected intraperitoneally (I.P.) with PolyI:C (Sigma-Aldrich, St. Louis, Mo., P-0913, 250 μg/dose) at indicated time points (3 times daily, on alternate days) to induce Cre-mediated LoxP recombination. Pups were randomly divided into two groups as follows: In the first group, the pups were injected with PolyI:C at P 2, 4, 6 injection time points. In the second group, pups were injected at P 4, 6, 8 points. Postnatal day 2 PolyI:C-injected mice were termed “P2-induced mice”, and postnatal day 4-injected mice were termed “P4-induced mice”. Prior to P10, pups in all groups were grossly normal in development. After P11, the PolyI:C injected recombinant Bmpr1a mutant mice depicted visible signs of growth retardation in comparison with the paired controls (data not shown). While P2- and P4-induced mice both exhibited Bmpr1a gene deletion, P2-induced mice depicted greater and more permanent HF Bmpr1a gene mutation effects, as will be shown, in particular, in subsequent examples 5 and 6.

### Example 3

The efficiency of the murine Mx1-Cre line in mediating LoxP-dependent DNA excision in the Bmpr1a gene in hair follicles (HFs) was determined by using a murine hybrid cross between the previously described Bmpr1a Mx1-Cre knockout mouse and a ZIEG reporter mouse. The hybrid mouse was selected because it permitted assessment of the extent of recombination occurring following administration of the recombination activator PolyI:C described in example 2 above.

The Z/EG reporter mouse was made by introduction of a Z/EG expression vector into R1 ES cells utilizing standard genetic engineering technology. This mouse was designated as “Z/EG” because it expressed both LacZ and enhanced green protein (GFP) reporters. This Z/EG double reporter mouse initially expressed the LacZ gene that encodes the β-galactosidase enzyme, driven by a ubiquitously active promoter, throughout embryonic and adult stages.

In the hybrid Mx1-Cre Z/EG reporter mouse, the LacZ indicator gene was flanked with LoxP sites. When the LoxP-flanked LacZ gene was deleted by the Cre enzyme in the hybrid mouse, expression of the second reporter, GFP, was activated. Cre recombinase activity and LacZ excision was triggered by postnatal injection of the recombination activator, PolyI:C. Thus, the presence of LacZ gene expression in cells, as indicated by X-gal staining, indicated the pre-DNA-excision state. In contrast, GFP expression represented the post-DNA-excision state.

As such, PolyI:C induced genetic recombination in the LoxP-flanked Bmpr1a gene of the hybrid Mx1-Cre Z/EG reporter mouse. Recombination at the Bmpr1a gene locus was detectable by loss of LacZ reporter expression and gain of GFP reporter expression in the Z/EG double reporter mouse.

### Example 4

Protocols for analyzing recombination in HF cells, through deletion of the LacZ expression and induction of GFP expression were used throughout subsequent examples. These protocols include the following:

LacZ gene expression of the β-galactosidase enzyme was detected by substrate staining. One suitable β-galactosidase substrate is X-gal, a 5-bromo-4-chloro-3-indolyl-β-D-galactopyranoside substrate (Sigma-Aldrich, St. Louis, Mo., FW=408.6, B4252), chemically characterized as an indole derivative. In X-gal staining, formalin-fixed skin was exposed to X-gal solution. After PBS wash, sections were counterstained with Nuclear-Fast-Red (Sigma-Aldrich, St. Louis, Mo., N-020). Non-expression of LacZ served as an indicator of Bmpr1a gene excision.

In addition to absence of expression of the LacZ reporter, GFP expression is indicative of successful Bmpr1a gene targeting. For GFP staining (Clonetech, Palo Alto, Calif.), skin was fixed in zinc formalin overnight, PBS washed, and immersed in 30% sucrose in PBS at room temperature overnight. On the second day, the skin was embedded in an OCT solution (ornithine carbamyltransferase, Miles Diagnostics, Inc., Elkhart, Ind.) and snap-frozen, then sliced into 8*m thickness sections, mounted with DAPI blue fluorescent counter stain, and prepared for imaging.

Skins were collected from post-excision mice on day 15 or day 40 following PolyI:C injection. These skins were GFP and X-gal stained as described. The slides were then air dried (1 hr, RT) and mounted with DAPI (4′-6-diamidino-2-phenylidole, dihydrochloride) blue fluorescent counterstain (InnoGenex, San Ramon, Calif.). DAPI preferentially stains double-stranded DNA, attaching to adenine-thymine (AT) clusters in the DNA minor groove. DAPI stains nuclei, with little or no cytoplasmic staining. After DAPI counterstaining, slides were then ready for imaging. Dorsal and ventral skins were collected at P15 (within the first cycle) or P40 (within the second cycle) for comparison.

### Example 5

Hair follicle regions of P2-PolyI:C-induced mice were compared to P4-induced mice in Z/EG double reporter mice to determine the permanence or transientness of Bmpr1a mutation induced. Hair loss and hair growth in P2-induced mice was examined to characterize phenotypic abnormalities observed in Bmpr1a mutant mice as compared to Wt mice. As will be shown, it was determined that P2-induced mice had greater hair loss and hair growth abnormalities than P4-induced mice. GFP and LacZ expression patterns indicated the degree of conversion from Wt to Bmpr1a mutant genotypic expression. P2-induced mice exhibited permanent Bmpr1a mutant HF conversion. This permanence is contrasted to the temporary, transient mutant HF conversion observed in HF tissue of P4-induced mice, as presented in the subsequent example.

Hybrid pups from the cross between Mx1-Cre⁺Bmpr1a^{fx/fx} mutant mice and Z/EG reporter mice were assayed by histochemical staining procedures following PolyI:C injections. P2-induced mice were then characterized with respect to HF phenotypic abnormalities expressed.

Isolated indicator bone marrow cells from pups were examined by flow cytometric assay to determine the degree of successfully induced Bmpr1a mutation. Bone marrow cells were solely used as indicators of Bmpr1a mutation rate among cells, as indicative of corresponding HFSC mutation rates. Staining results revealed that only 50% of bone marrow cells generally demonstrated successful targeting of the Bmpr1a gene as evidenced by expression of GFP following a single injection of PolyI:C activator (data not shown). However, multiple injections of PolyI:C yielded higher efficiencies of recombination and Bmpr1a mutant cell expression. For this reason, it was determined that three sets of PolyI:C injections on alternate days were to be utilized to obtain the P2-induced and P4-induced mice in the injection protocol, as described previously.

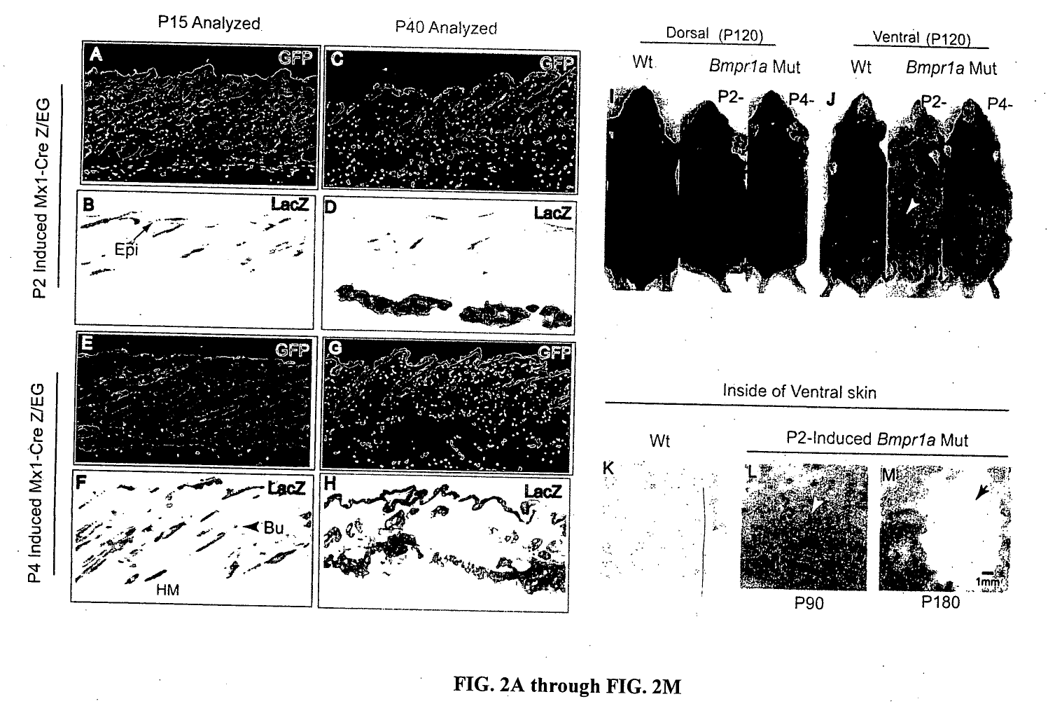

Following injection of P-2-induced mice, the Mx1-Cre-dependent DNA recombination efficiency was analyzed in HFs and epidermis of the Wt and Bmpr1a mutant Z/EG reporter mice, as shown in FIG. 2. The presence of the GFP reporter signal (green) indicated successful Bmpr1a receptor gene targeting while the LacZ reporter signal (blue) represented un-targeted cells.

The HF cells in the ventral skin of the P2-induced mice, in which injections started 2 days after birth, converted from LacZ to GFP-expression regardless of whether skins were analyzed on day P15 or day P40, as shown in results depicted in FIGS. 2A through 2D. GFP was expressed in almost all the HFs and in the epidermis of the P2-induced mice, as shown in FIGS. 2A and 2C. Correspondingly, GFP was also maintained in the majority of the HFs and epidermis when analyzed on P40, as shown in FIG. 2C. Only a small number of the HF cells in the dorsal skin converted from LacZ to GFP expression when analyzed on P15 (data on file). The clonality of P2-induced HF mutation is suggested by comparisons of GFP expression staining patterns on P40 vs. P15. Visual examination depicted more extensive staining across the length of the hair shaft in P40, as shown in FIG. 2C, as compared to truncated staining patterns in P15 sections, as shown in FIG. 2C.

In addition to gain of GFP reporter expression, loss of LacZ reporter expression (blue) also indicated successful deletion of the LacZ gene, as shown in FIGS. 2B and 2D. Sporadic LacZ signals indicated that some HF cells on P15 or P40 still remained LacZ gene untargeted. In contrast, significant LacZ staining was observed below the HF-containing region of the epidermis in P40 analyzed tissues, as shown in FIG. 2D.

Conversion from LacZ to GFP expression in P2-induced mice indicated successful excision of the Bmpr1a receptor gene in the conditional Cre-Lox-mediated knockout process, induced by PolyI:C injection

### Example 6

P4 PolyI:C-injected mice were examined and compared with the P2-injected mice of Example 5. In P4-induced mice, it was determined that the Bmpr1a receptor gene was incompletely and transiently knocked out by the recombination activator, in contrast to the permanent knockout observed P2-induced mice.

P4-induced Z/EG reporter mice were injected with PolyI:C on the fourth day after birth, then analyzed at P15 and P40, as shown in FIGS. 2E-2F and FIGS. 2G-2H respectively. Unlike the highly prevalent GFP staining pattern of HFs observed in P2-induced Z/EG reporter mice above, GFP expression was detected in only some of the HFs and the epidermis in the P4-induced reporter mice, as shown in FIG. 2E. A lesser degree of knockout also existed in P-4 induced mice as compared to P-2 induced mice, as shown in FIGS. 2E-2H, as compared to FIGS. 2A-2D. Typical GFP staining patterns at P15 are shown in FIG. 2E, where GFP staining was observed in only some HFs and in the epidermis of P4-induced reporter mice. As anticipated, HFs and epidermis of P4-induced mice lacked significant LacZ expression, as shown in FIG. 2F. However, consistent with lesser knockout in P4-induced compared to P2-induced mice, LacZ expression was greater in P4 mice than in P2 mice, as shown in FIGS. 2F and 2B. Note that in some HFs, loss of LacZ expression was seen throughout the HFs but not in the bulge area, as also depicted in FIG. 2F.

Unlike P2-induced mice, the P4-induced mice did not depict complete and permanent conversion from LacZ to GFP expression in the ventral HFs. Particularly, in a few HFs, bulge area cells retained LacZ expression at P15, as illustrated in FIG. 2F, while cells in the remaining structure and the overlying epidermis lost LacZ expression. This was a transient conversion, and it was no longer observed at the P40 time point, as shown in FIGS. 2G and 2H. At the P40 time point, GFP expression was lost in the majority of HFs and in the epidermis in the P4-induced mice, as depicted in FIG. 2G, while LacZ expression was detected in the majority of HFs and the epidermis, as shown in FIG. 2H. In P4-induced mice, the observation of incomplete and transient conversion from LacZ to GFP expression in HFs led to the conclusion that P4-induction with PolyI:C yielded lesser and transient Bmpr1a mutation in comparison to P2-induction which yielded widespread and permanent HF Bmpr1a mutation.

Successful conditional inactivation of the Bmpr1a gene in HF stem cells in the Bmpr1a mutant mouse, demonstrated that the mutant Bmpr1a^{fx/fx} mouse genotype expressed the phenotypic trait of permanent hair loss in both P2-PolyI:C-induced and P4-induced mice. Dorsal and ventral skin and hair staining of Wt, P2-, and P4-induced Bmpr1a mutant mice at the P120 time point are shown in FIGS. 2I-2J. P120 results obtained were similar to those observed at the earlier P40 time point. In the P2-induced group, mice lost hair in both dorsal and ventral skin at P120, but the loss was more severe in ventral skin, as shown in FIG. 2I as compared to FIG. 2J. In the P4-induced group, ventral hair loss was still present, but dorsal hair development appeared to be less affected (not shown). Thus, the striking permanent hair loss observed in the ventral skin of the P2-induced mice at P120, indicated that inactivation of Bmpr1a occurred at the stem cell level.

At P120, it was concluded that P2 induction resulted in complete, permanent Bmpr1a hair follicle mutation, whereas P4 induction resulted in incomplete, transient Bmpr1a mutation. This is particularly evident in the ventral whole mouse photos exhibited in FIG. 2J, where P2-induced mice exhibited the greatest hair loss phenotypic defect, and where P4-induced mice exhibited lesser hair loss.

In contrast to hair loss, hair growth was found to be cycle-dependent in PolyI:C injected Bmpr1a mutant mice, with the first cycle initiated around birth, and the second cycle initiated between P20 and P30 postnatal days. Between days 35 and 40 after birth, hair growth recovered in the majority of skin in all mice except for ventral skin in the P2-induced mice (data not shown). This finding was presumably due to secondary HF growth with the newly formed hair being regenerated from untargeted stem cells because of the partial Cre activity.

In summary, it was observed that P2-induced and P4-induced mutant Bmpr1a^{fx/fx} mice both exhibited hair loss, with the P2-induced mice exhibiting greater severity of hair loss in both ventral and dorsal skin. P2-induced mice also demonstrated concomitant abnormalities in hair growth, particularly in ventral skin. In general, the earlier PolyI:C was injected postnatally, the more severe the hair loss. This may reflect the early anagen time window, in which the bulge stem cells were activated. This indicates that recombination targeting of Bmpr1a gene mutation in an HFSC earlier in development yields a greater effect.

Taken together with P2-induced mouse results described previously, these P4-induced mouse results were consistent with the following conclusion: A critical time window existed before the pivotal P4 time point. During this pre-P4 time period, HFSCs in the ventral skin exhibited highest sensitivity to PolyI:C-induced, interferon-dependent, Cre mediated DNA gene excision. As such, in the PolyI:C P2-induced group of mice, Cre-mediated LacZ inactivation of the Bmpr1a receptor gene occurred at the stem cell level in most of the ventral HFs due to the activated (cycling) state of bulge stem cells. This explanation accounts for the widespread pattern of GFP tissue expression in P2-induced tissue, not only in HFs but also in the overlying epidermis, as shown in FIGS. 2A and 2C.

However, in the P4-induced group of mice, it was concluded that Cre-mediated LacZ inactivation only occurred in some of the transient amplifying (TA) stem cells or progenitor cells of ventral HFs. This would explain the loss of GFP expression between P15 and P40, as shown in FIGS. 2E and 2G. Since Cre-mediated DNA excision occurred only in the activated stem cells, a low level of expression of interferon receptor in arrested stem cells may account for the observed restriction to activated cells.

While the GFP marker was prominently and permanently expressed in HFs of P2-induced mice, GFP was only transiently detected in some HFs of P4-induced mice. Therefore, P2-induced mice yielded greater efficiency of targeted Bmpr1a gene excision in HFSCs than P4-induced mice, as demonstrated by GFP and LacZ expression patterns at P-15 and P-40 time points. It was concluded from the foregoing results that Bmpr1a gene activity was entirely knocked out in P2-induced mutant mice, but not in P4-induced mice.

### Example 7

While GFP and LacZ marker studies indirectly assess the presence of Bmpr1a mutations in hair follicles, polymerase chain reaction (PCR) analysis can be performed to directly analyze the presence of Bmpr1a mutant genes and related fragments in P2-induced Bmpr1a mutant mouse tissue as compared to Wt tissue. PCR analysis was performed upon DNA obtained from Mx1-Cre Bmpr1a mutant mice following PolyI:C induced Bmpr1a gene excision (e.g., P-2 or P-4) to determine whether anticipated Bmpr1a genetic recombination had occurred. Tails were collected from the Bmpr1a mutant mice on day 21 or at the time of termination to extract genomic DNA. Genotyping by PCR assay was executed utilizing different combinations of primers:

| | | |
|---|---|---|
| Pr1: | GCAGCTGCTGCTGCAGCCTCC; | (SEQ ID NO: 13) |
| Pr2: | TGGCTACAATTTGTCTCATGC; | (SEQ ID NO. 14) |
| Pr3: | GGTTTGGATCTTAACCTTAGG; | (SEQ ID NO. 15) |
| Pr4: | TACCTGGAAAATGCTTCTGT; | (SEQ ID NO. 16) |
| and, | | |
| Pr5: | TGATCTCCGGTATTGAAACT. | (SEQ ID NO. 17) |

Primers Pr1 and Pr2 amplified 230 bp and 150 bp products and were specific for the floxed and wild-type alleles, respectively. The combination of primers Pr2 and Pr3 amplified a 180 bp product specific for the targeted Bmpr1a allele after PolyI:C-induced Cre-mediated Lox-site specific recombination. Pr4 and Pr5 primers were used to identify Mx1-Cre with an 808 bp product in Cre-positive mice. Using PCR analysis, the pre-excision wild-type (2500 bp=intact) and post-excision mutant (180 bp=truncated) states of the Bmpr1a-containing Exon2 were determined.

The post-excision truncated Exon 2 nucleic acid sequence (SEQ. ID. NO. 3) of the Bmpr1a mutant mouse encoded amino acids numbered 24-76 (SEQ. ID. NO. 6) of the Wt intact Bmpr1a polypeptide molecule (SEQ. ID. NO. 4) which contained 478 amino acids. Thus, it was demonstrated by PCR analysis that successful recombination occurred at Lox sites flanking the Bmpr1a gene of the Mx1-Cre⁺Bmpr1a^{fx/fx} Bmpr1a mutant mouse. This recombination event resulted in PolyI:C injection-induced excision of Exon2 of the Brmpr1a gene. Expression of the Exon 2-excised Bmpr1a gene resulted in an encoded inactive Bmpr1a receptor polypeptide (SEQ. ID. NO. 5). Thus, PCR analysis confirmed the GFP and LacZ staining results that depicted inactivation of the Bmpr1a gene in P2-induced mice.

### Example 8

PolyI:C-induced inactivation of the Bmpr1a receptor in HFSCs in the Bmpr1a P2-induced mutant mouse led to abnormal growth of melanocyte stem cells and HF tumors. These secondary phenotypic abnormalities resulted from aberrant hair follicle growth. HF tumors, classified as matricomass, were observed in P2-induced, but not in P4-induced Bmpr1a mutant mice.

Progression from cell morphology exhibiting black, speckled depositions to tumor formation was observed in P0-induced to P2-induced Bmpr1a mutant mice. This tumor transformation was not observed in P4-induced mice. A large number of black, speckled depositions was found beneath the ventral skin in the P2-induced Bmpr1a mutant mice around P90, as shown in FIG. 2L. These speckled depositions were due to an accumulation of melanin, which indicates that the melanocyte stem cells were also targeted by the mutation. Some of these targeted cells progressed to become visible, solid tumors (with diameter>1 cm) after 6 months (P180), as shown in FIG. 2M. The number and size of the speckled depositions increased and merged together, as the mice aged. These speckles were, however, rarely observed in the dorsal skin of P2-induced mice.

In contrast, almost no speckled depositions were found in either dorsal or ventral skin in the P4-induced mice (data on file). As expected, injecting PolyI:C at P0 or P1 resulted in more numerous speckles and tumor formation than injection at P4 (data on file).

In conclusion, results indicated that when the Bmpr1a receptor mutation was induced in HFSCs by PolyI:C at the time period from P0 to P2, tumors, as indicia of permanent hair defects, were observed. In contrast, when the Bmpr1a mutation was induced later, at P4, in either progenitor or TA stem cells, tumors were infrequent, consistent with the view that defects induced were transient. As such, permanent tumor induction depended upon injection of the PolyI:C recombination activator at 0 to 2 days after birth; however, injection at 4 days resulted only in transient hair dysplasia.

### Example 9

Additional hair follicle structural support was obtained for the conclusion that inactivation of the Bmpr1a gene in Bmpr1a mutant mice led to dysplastic hair follicles. Such gene inactivation may ultimately lead to formation of matricomas-type tumors in the mutant Bmpr1a mouse. In addition, comparison of P2-induction with P4-induction indicated a difference in the types of cells targeted.

Hair follicle sections from Wt, P2-, and P4-induced Bmpr1a mutant mice were analyzed at P15, 20, 25, and 30 time points. Histological analysis of hair follicle morphology was conducted, whereby tissue sections were stained with hematoxylin and eosin.

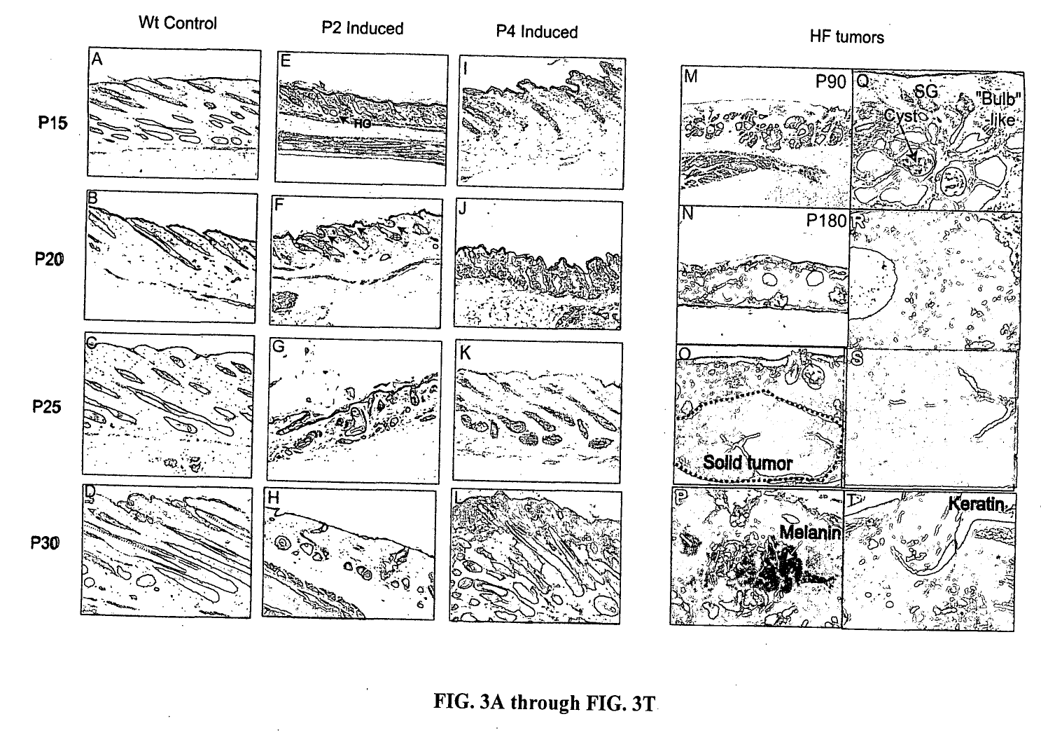

In P2-induced mutant mice, multiple, newly formed hair germs (HG) were observed surrounding the original HFs in these mice, as shown in FIGS. 3E-3H. Mutant hair germs appeared as shortened HF structures below the epidermal surface. An exemplary hair germ is depicted at the arrow in FIG. 3E. HF dysplasia progresses from P20 to P25 to P30, where hair germ and HF structure becomes increasingly abnormal, as depicted in FIGS. 3F, 3G, and 3H respectively.

Additionally, in P2-induced mice, the permanent segment of the HF structures appeared to be grossly normal at P15 and P20, as shown respectively in FIGS. 3E and 3F, in comparison with the Wt HF, as shown in FIGS. 3A and 3B. However, the cycling segment of HF structure was impaired, with one permanent segment of the original HF connected to multiple, abnormal hair germ (HG)-like structures, as shown in FIGS. 3E-3H. The outgrowth of these structures continued to increase in both number and size to form “bulb”-like structures as the mice aged. When P2-induced mouse skin was analyzed at P90, many “bulb”-like structures were found to emerge from a large, central cyst, as shown in FIGS. 3M and 3Q.

This P2-induced mutant pattern contrasts with the extended single HF structures seen in the Wt skin, as shown in FIGS. 3A-3D. In Wt control skin, the hair follicle lengthens during timepoints from P15 to P30 to form the intact Wt HF structure, as depicted in FIG. 3D. The contrast between the P2-induced truncated HF structure and Wt elongated HF structure is particularly evident in FIG. 3H, as compared to FIG. 3D. However, P2-induced mutant HF structures are also distinguishable from Wt HF structures at P30, P20, and P15, as evidenced by comparisons between FIGS. 3G and 3C, FIGS. 3F and 3B, and FIGS. 3E and 3A, respectively.

In the P4-induced mice, the HF structures exhibited abnormal appearance before the P25 time point (i.e., P15 and P20), as shown in FIGS. 3I and 3J. These abnormal, truncated HF structures are distinguishable morphologically from the normal HF structure in control Wt HFs, as shown in FIGS. 3A and 3B, respectively. In contrast, on and after the P25 time point, these P4-induced mutant mice exhibited a HF structure that became more normal in phenotype. In these P4 mice, new hair developed, and external hair grew at P25 and P30, as shown in FIGS. 3K and 3L respectively, in comparison with corresponding FIGS. 3C and 3D. These findings were consistent with the previously presented view, that in P4-induced mice, the inactivation of Bmpr1a occurred in the progenitor cells, rather than in the stem cells, resulting in only a temporary interruption of HF growth. The remaining (quiescent) stem cells with untargeted Bmpr1a genes then generated a completely new HF in the subsequent HF cycle, as shown in FIGS. 3I-3L. Thus, it was concluded that P4-induced mice exhibited fewer HF phenotypic abnormalities than P2-induced mice.

### Example 10

Following the detection and characterization of abnormal HF structures present in P2-induced mice in the foregoing examples, it was inquired whether tumorigenesis might be triggered in P2-induced mice as well. Sections of hair follicle tissue in the P2-induced mice at P90 and P180 time points were examined. The abnormal bulb-like structures discovered in P2-induced mice had morphological features typical of human matricomas, a hamartoma with multiple abortive HFs opening into a central cyst, as shown in FIGS. 3M, 3N, 3Q, and 3R. Within the cysts of some tumors, melanin deposits accumulated, which accounted for the black, speckled depositions, as shown in FIGS. 3P and 3T. Each abortive HF produced keratinous material but did not form a hair shaft, reflecting the inappropriate differentiation in these matricomass. Thus, P2-induced mice exhibited extensive phenotypic tumor-related abnormalities, manifested as a result of Bmpr1a gene deletion.

The tumorous HFs revealed no dermal papilla (DP) structures (data on file), as revealed by trichrome staining, which was used to distinguish dermal papilla from hair matrix (HM) cells. This finding suggested that in P2-induced Bmpr1a mutant mice, when the hair matrix cells no longer recognized the BMP signal, a fraction of these cells aberrantly formed solid tumors over time, such as matricomass, as shown in FIG. 3O. Tumor sections were analyzed by histological staining to reveal thousands of abortive HFs, as shown in FIGS. 3O and 3S.

Typical HF tumors with the features of matricomas are shown in FIGS. 3M-3T. Tumor at the P90 time point is shown in FIG. 3M, and tumor at P180 of the P2-induced Bmpr1a mutant mice is shown in FIG. 3N. Comparison of tumors at P90 and P180 depict that increases in cell number and tumor size occur with the passage of time.

Enlarged views of tumor sections, depicting occupation of HF regions by increasing numbers of tumor cells, are displayed in FIGS. 3Q and 3R. It is evident that the cyst in FIG. 3Q was surrounded by multiple “bulb”-like structures, indicative of tumorous morphology. A section of a representative solid tumor is shown in FIG. 3O, and a tumor close-up view is shown in FIG. 3S. Thousands of “bulb”-like structures, shown in FIG. 3Q, were estimated to exist in a typical solid HF tumor, as shown, for example, in FIGS. 3O and 3S. Melanin pigment deposition and accumulation in HF tumors is shown in FIG. 3P. The white arrow indicates the presence of the ubiquitous black melanin pigment. In addition, keratin, a fibrous epidermal protein, accumulated in the cyst area, as shown in FIG. 3T.

In conclusion, while BMP signaling controlled expansion and activation of normal Wt HF stem cells, the disruption of BMP signaling in hair matrix cells of Bmpr1a mutant mice caused HF abnormalities, often leading to subsequent matricomas formation. This rationale was supported by the observed occurrence of matricomas in P2-induced mutant mice.

### Example 11

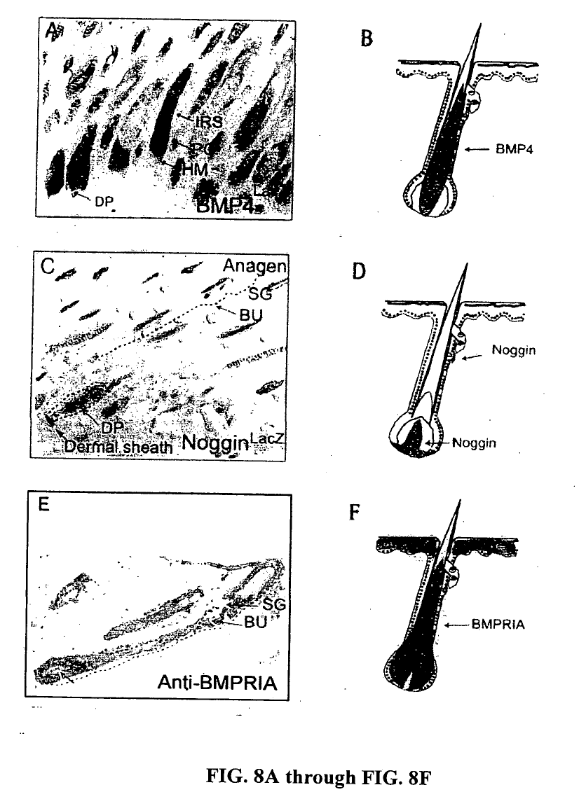

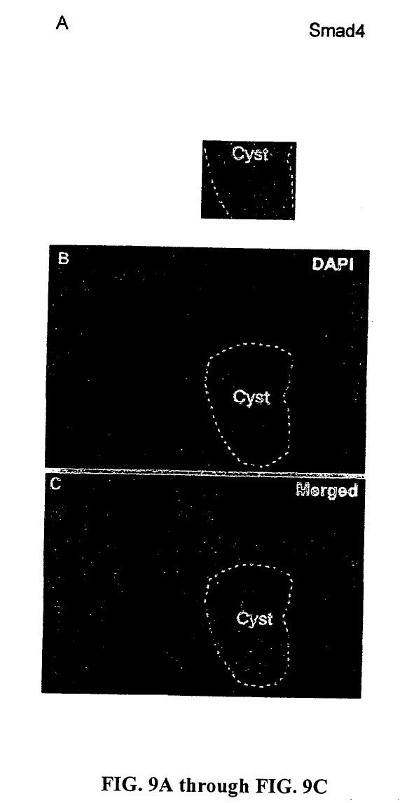

Analyses of hair follicle gene expression patterns were conducted for BMP4, Noggin, Bmpr1a, and ancillary gene expression markers (e.g., LacZ) in BMP4-LacZ and Noggin-LacZ knock-in mice, as shown in FIG. 8. Hair follicle sections of BMP4-LacZ and Noggin-LacZ knock-in mice were stained with (-galactosidase to determine LacZ expression (blue) which reflects BMP or Noggin mRNA expression in knock-in mouse tissue, as shown in FIGS. 8A-8D. BMP or Noggin mRNA expression is detected, rather than protein expression, because the tissue from BMP-LacZ or Noggin-LacZ knock-in mice produces (-galactosidase mRNA as a marker that permitted BMP or Noggin gene distribution analysis, respectively. Fast Red was used as a counterstain.

BMP4 was expressed widely in a variety of HF structures of BMP4-LacZ murine hair follicle tissue, including dermal papilla (DP), hair matrix (HM), precortex (PC), and inner root sheath (IRS) areas, as shown in FIG. 8A. Colorimetric visual examination of the distribution of BMP4 activity (blue) in hair follicle tissue revealed that a BMP4 activity or presence existed along the hair shaft. This BMP4 activity gradient is depicted diagramatically by the green coloration in the drawing of FIG. 8B.

Examination of the distribution of Noggin activity (blue) in HF tissue at anagen phase depicts that Noggin activity was highest in the dermal papilla (DP) and dermal sheath (DS) region along the bottom of the hair bulb of Noggin-LacZ mice, as shown in FIG. 8C. Noggin was also detected in the bulge (BU) area, where HFSCs were determined to reside. The Noggin distribution pattern (blue) is illustrated diagrammatically in the drawing of FIG. 8D, where Noggin is shown to be localized in the hair bulb (HB) and bulge (BU) regions.

In summary, it was determined that BMP4 activity was widespread and existed throughout HF structures, including dermal papilla (DP), hair matrix (HM), precortex (PC), and inner root sheath (IRS) areas. In contrast, Noggin activity was more restricted to hair bulb (HB) and bulge (BU) regions, where HFSCs resided. Based upon the differing distribution patterns of BMP and Noggin in hair follicle tissue in BMP4-LacZ and Noggin-LacZ mice respectively, the view was presented that BMP and Noggin were antagonistic, opposing signals. Further, these competitive signals may play a critical role in induction of HF proliferation, differentiation, and apoptosis within zones along the hair follicle.

### Example 12

Characterization of BMP and Noggin activity distributions in the hair follicle, as presented in example 11, suggested that a regulator molecule, such as Bmpr1a receptor, might be involved in the zonal regulation of HF proliferation, differentiation, and apoptosis. To investigate this hypothesis, anti-Bmpr1a antibody staining of the hair follicle was conducted. Bmpr1a was expressed throughout the entire HF region, including the bulge area, as shown in FIG. 8E. The contours of the hair follicle structure itself are readily apparent in this photo, as indicated by the overlying dotted lines. Arrows indicate bulge (Bu) and sebaceous gland (SG) areas, as well as the hair bulb region. The distribution of Bmpr1a along the HF appeared to be slightly more extensive than either BMP or Noggin activity distribution patterns, as shown previously in FIGS. 8A and 8C. The Bmpr1a distribution pattern, as diagrammatically illustrated in FIG. 8F, was approximately superimposable on a combination of BMP plus Noggin distribution patterns, as shown in FIGS. 8B and 8D.

These observations supported the view that HF cell development might be influenced by modulation of BMP4 and Noggin binding to the Bmpr1a receptors across the hair follicle structure.

### Example 13

As a continuation of the foregoing examples, demonstrating that Noggin-mediated modulation of BMP activity is involved in the regulation of HF development, Noggin distribution patterns in hair follicle cells in both anagen and catagen phases were examined using Noggin-LacZ knock-in mice, as shown in FIGS. 1B-H. Results supported the view that Noggin played a role in regulating BMP activity which, in turn, functioned to establish zones of self-renewal, proliferation, and differentiation in the hair follicle. High Noggin levels of expression in anagen resulted in HF cell activation and proliferation in the bulge and dermal papilla regions.

Procedurally, skin sections containing hair follicle cells in anagen and catagen phases were extracted from Noggin-LacZ mice and stained for the presence of (-galactosidase. Noggin expression in anagen phase HF cells was determined to be present in the bulge (Bu) region, as depicted by LacZ staining (blue) in FIG. 1B. The bulge's position was identified relative to its location adjacent to the arrector pili muscle (APM) beneath the sebaceous gland (SG), as shown more distinctly in the close-up view of FIG. 1C. As previously determined, HFSCs were found to reside in the bulge region. High Noggin expression levels were found in the late anagen phase of HF cells, as shown in FIG. 1B.

Thus, in early anagen, the high level of Noggin expression resulted in low BMP activity in the bulge (Bu) region, as illustrated diagrammatically in the left panel of FIG. 1J. A BMP densitometer trace, depicting focused BMP expression in the center region of the hair shaft, is shown in FIG. 1K, right panel. Higher expression of Noggin in the dermal papilla (DP) inhibited the activity of BMP in the hair matrix region. This Noggin expression in the dermal papilla led to decreased BMP activity.

In contrast, Noggin was down-regulated in both the dermal papilla (DP) and bulge (Bu) regions during the catagen phase (P25), as shown respectively in FIGS. 1D and 1E. In catagen, lack of blue staining indicated, at the black arrows, that Noggin levels were low in both dermal papilla and bulge areas. In catagen, lower levels of Noggin expression in hair matrix (HM) and bulge (Bu) regions led to increased BMP activity, as illustrated diagramatically in FIG. 1K. A BMP densitometer trace, depicting wide-ranged BMP expression across the hair shaft, including the hair matrix region (HM) is shown in FIG. 1J, right panel.

The relationship between BMP4 mRNA and Noggin mRNA distribution patterns across the length of the hair follicle in anagen phase is diagrammatically illustrated in FIG. 1I. The blue-colored regions, such as those in the dermal papilla and dermal sheath areas, depict Noggin activity. The green regions depict extensive BMP4 activity, extending through hair matrix, precortex, inner root sheath, hair shaft, outer root sheath, and bulge areas.

Taken together, histological staining patterns obtained support the view that Noggin levels vary along the HF axis and that an interacting activity gradient is established based on relative expression levels of Noggin and BMP. During the anagen phase, Noggin levels were increased both in HF cells of hair matrix (HM) and bulge (Bu) regions, where self-renewal and proliferation activity was also increased, as shown in FIG. 1J. Results indicated that decreased expression of Noggin in the bulge led to the maintenance of a quiescent stem cell (arrested stem cell); and, in addition, down-regulation of Noggin in the dermal papilla resulted in a significantly higher BMP activity in the hair matrix region. In contrast, in the catagen phase, Noggin levels decreased in hair matrix and bulge areas, with concomitant increase in BMP activity and decrease in proliferation, as shown in FIG. 1K. Thus, modulation of Noggin levels during anagen and catagen phases impacted the capabilities of hair follicle cells to undergo self-renewal and proliferation.

Similarly, graphical illustrations of relative expression levels for Noggin and BMP4 spanning across the length of the hair structure in anagen and catagen phases are shown in FIGS. 1J-1K, respectively. These illustrations summarize results observed in HF tissues of Noggin and BMP knock-in mice, as previously depicted in FIG. 8 and discussed in previous examples. In the anagen phase, as illustrated graphically in FIG. 1J, the following three observations were made: Low levels of BMP activity in the bulge corresponded to the self-renewal zone of HF stem cells. Relatively lower levels of BMP activity in the HM region allowed cells to undergo proliferation in a proliferation zone. Higher levels of BMP favored cell differentiation in a differentiation zone.

In conclusion, results supported the view that BMP-dependent signaling was required for dermal papilla maintenance, and Noggin expression in dermal papilla and bulge regions was essential for bulge stem cell activation. Expression of matrix signals required for dermal papilla maintenance was BMP-dependent, and BMP signaling through Bmpr1a provided a restriction signal for arrested stem cell activation. Noggin played an essential role in activation of arrested stem cells by overriding the BMP restriction signal. This conclusion is supported by findings that neutralization of BMP activity, through over-expression of Noggin, led to induction of the anagen phase, while targeted inactivation of Noggin caused significant retardation of HF induction. The initiation trigger for HFSC activation in the bulge depended upon the relative Noggin vs. BMP expression levels, regardless of the cellular source of the Noggin signal (i.e., dermal papilla or bulge).

### Example 14

Confirmatory evidence for Noggin/BMP regulatory control of HF cells, described in the previous examples, was subsequently found by investigating HF intracellular distribution patterns for Smad4, during anaphase, catagen, and telogen phases, as shown in FIGS. 1F-1H. Specifically, it was asked whether increased Noggin expression led to Smad4 cytoplasmic localization, resulting in subsequent HFSC activation. It was determined herein that Noggin binding to Bmpr1a receptor on HFSCs resulted in Smad4 cytoplasmic signaling in anaphase, leading to HFSC activation.

Noggin signaling and its potential induction of cellular expression of Smad4, the downstream transcriptional factor mediating BMP/TGFP signals, was examined by investigating the Smad4 marker staining distribution pattern in hair follicle (HF) tissue in mouse skin. In this procedure, skin sections derived from the Bmpr1a mutant mice were first stained with anti-Smad4 primary antibody. Next, secondary antibody was added that recognized the primary anti-Smad4 antibody (e.g., goat anti-mouse antibody) and was conjugated to the Alexa Fluor 647 marker (red) (Molecular Probes, Inc., Eugene, Oreg.). As previously, DAPI (light blue) was the counterstain used.

In Noggin-LacZ knock-in mutant mice, in which Noggin tissue activity levels were increased above Wt, cellular localization of Smad4 in HFs in early anagen, late anagen and telogen phases was examined, as depicted in FIGS. 1F-1H. Significantly, in the early anagen phase, high Noggin expression levels induced cytoplasmic localization of Smad4 signal (red) in the bulge, as shown in FIG. 1F. Smad4 was also detected in the cytoplasm of matrix cells in late anagen phase, as shown in FIG. 1G. In contrast, nuclear localization of Smad4 was found in HFSCs in the bulge region in telogen phase, as shown in FIG. 1H, and in the precortex cells in late anagen, as shown in FIG. 1G.

Smad4 staining results in Noggin mutant mice confirmed the view that Noggin regulated self-renewal and proliferation through cytoplasmic location of Smad4 during early anagen phase. Decrease of Noggin activity during late anagen phase apparently induced nuclear localization of Smad4 in the precortex cells, as shown in FIG. 1G. However, in telogen, down regulation of Noggin expression in the bulge induced Smad4 localization in the nucleus of the HFSCs, as shown in FIG. 1H. Similar Smad4 nuclear localization occurred during catagen (data on file).

Taken together with results from previous examples, results described herein support that Noggin inhibited BMP-mediated activity in HFSCs in anagen phase, resulting in increased HFSC proliferation and self-renewal. Cytoplasmic localization of Smad4 was determined to be an early indicator of Noggin-mediated HFSC activation, where Smad4 is the downstream transcriptional factor resulting from BMP/TGFβ signals. However, when Noggin was down-regulated during catagen and telogen phases, BMP activity increased, and Smad4 localized in the HFSC nucleus.

### Example 15

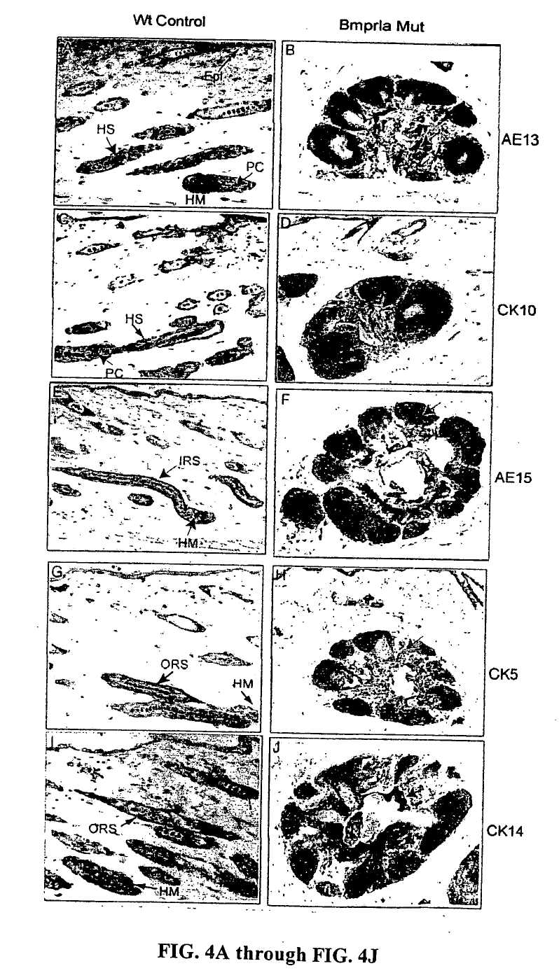

Differentiation impairment in the hair follicle (HF) tumorous tissue of the Bmpr1a mutant in comparison to the Wt mice was characterized by the presence, either increased or decreased, of differentiation markers in mutant tissue. Ventral skin sections derived from Wt control and the Bmpr1a mutant mice were each stained respectively for a variety of HF differentiation markers (AE13, AE15, CK5, CK10, and CK14), as shown in FIG. 4. Markers are proteins associated with discretely identified hair follicle structures, and these markers were detected utilizing immunofluorescent, immunoenzymatic, and histochemical staining methodologies.

These various differentiation markers were associated with different Wt hair follicle structures and permitted the characterization of Bmpr1a-induced mutational defects in hair follicle development. As such, CK 5, 10, and 14, and AE15 were found in Wt epidermis, with CK14 associated with epidermal progenitor cells. AE15 and CK 5 and 14 were found in the Wt outer root sheath (ORS), while AE15 was seen in the inner root sheath (IRS) structure. AE13 and CK10 were present in the hair shaft, while AE13 itself was also found in the precortex (PC) region. By examining distribution patterns of these differentiation markers, it was determined that Bmpr1a mutant mice possessed abnormal differentiation within their tumors and dysplastic HF structures.

The AE13 marker was detected in the hair shaft and precortex cells of Wt control HFs, as shown in FIG. 4A, and in lower numbers in the centers of cysts of dysplastic HF tumors in the abnormal HFs of Bmpr1a mutant mice, as shown in FIG. 4B. CK10 was detected in hair shaft and epidermis cells of Wt HFs, as shown in FIG. 4C, but barely detectable in the “bulb”-like structures in mutant HFs, as shown in FIG. 4D. HF tumors expressed relatively low numbers of cells bearing AE13 and CK10 markers in comparison to Wt tissue. This indicated that AE13 and CK10 markers for hair shaft, precortex, and epidermis cells were less expressed in tumorous tissue.

AE15, a keratin marker specific for both inner root sheath (IRS) and the epidermis of normal HFs, was detected in the IRS cells of Wt HFs, as shown in FIG. 4E. AE15 was also found in the subset of cells of the inner layer of each “bulb”-like structure of the tumorous mutant HFs, as shown in FIG. 4F. AE15-labeled cells appeared in increased numbers in tumorous tissue in comparison to Wt HF tissue. Thus, AE15, an IRS and epidermis-associated marker, was present in greater numbers of mutant tumor cells.

CK5 was detected in the outer root sheath (ORS) and the epidermis cells of Wt HFs, as shown in FIG. 4G, and in the cells of ORS-like and the cyst boundary in mutant mice, as shown in FIG. 4H. CK5 was detected in a subset of the outer layer of the “bulb”-like structure, adjacent to the AE15 positive cells, and in the boundary of the cyst, as shown in FIG. 4H. Since CK5 was present in relatively equal amounts in normal Wt and mutant tumor tissue, mutation apparently did not significantly impact CK5 marker distribution.

CK14 is a marker for epidermal progenitor cells and the outer root sheath (ORS) of the hair follicle. CK14 was detected in cells of the outer root sheath (ORS) and in the epidermis of Wt HFs, as shown in FIG. 4I. CK14 was also found in the cyst boundary of abnormal HFs of mutant mice, as shown in FIG. 4J. The CK14 distribution in the tumorous HF structure was similar to the CK5 distribution, and did not readily distinguish between Wt and tumor tissue.

Taken together, the foregoing results depicted the cumulative effect that blocking the BMP pathway in the hair follicle of the Bmpr I a mutant mice partially inhibited differentiation of the hair shaft, as indicated by the low number of cells expressing AE13 and CK10, as hair shaft markers, in comparison to Wt. This conclusion also was supported by the appearance of an increased number of cells in the mutant mouse expressed the inner root sheath (IRS) marker, AE15, in the tumorous HFs. Differentiation of the outer root sheath (ORS) seemed to be less affected by Bmpr1a mutation, as assessed by staining with CK5. These results suggested that in the normal HF, the presence of a BMP signal favored hair shaft fate differentiation rather than inner root sheath fate. This conclusion was consistent with nuclear-localization of Smad4, described previously, which also indicated that normal Wt HF cells responded to BMP signaling in the precortex region and not in the inner root sheath. Finally, it was evident that the HF structure was dramatically disrupted morphologically in mutant tumor regions. However, in spite of this HF structural disruption, each “bulb”-like structure in the mutant retained a certain degree of architectural organization, such as the relative position of inner and outer root sheath-like cells (i.e., IRS, ORS), and the deposition of keratin into the central cyst.

### Example 16

After characterization of differentiation impairment in Bmpr1a mutant mice in the previous example, examination of the distribution of proliferation markers permitted the investigation of expansions in the activated and transient amplifying HFSC populations relative to the arrested HFSC populations. Bmpr1a mutant and Wt hair follicle tissues were analyzed, as shown in FIG. 5. Hair follicle-containing skin sections derived from Wt and Bmpr1a mutant mice were stained with anti-BrdU, anti-Ki67, and anti-β1-integrin proliferation marker antibodies. These antibodies were used to determine the distribution of identifiable proliferating cell populations in HF areas including the bulge, the bottom region of HB, the base of “bulb”-like structures of HF tumors, and the cyst boundary of HF tumors. Staining by secondary antibody against the background of the foregoing primary antibodies alone was detected by AEC labeling (red), where the counterstain employed was hematoxylin (blue).

Arrested BrdU-LTR (BrdU Long-Term Retaining) cells (arrested stem cells) were found occasionally in the bulge of Wt HFs, as shown in FIG. 5A. The arrested stem cell population was significantly increased in the bulge area of abnormal mutant HFs, as shown in FIG. 5B. In Bmpr1a mutants, the arrested stem cell number was increased an average of 10-fold at P70. Arrested HFSCs, characterized as nonproliferating BrdU-LTR cells, were occasionally found in the Wt HF bulge areas, but these HFSCs were significantly increased in the bulge area of mutant HFs.

After localizing the arrested stem cells to the bulge region in Wt and mutant HF tissue, activated and transient amplifying HFSCs were subsequently examined. While the activated stage HFSCs and the transient amplifying stage HFSCs in Wt control mice were both identifiably pulse-labeled with BrdU, this initial labeling was short-lived and lost over time as these Wt cell subpopulations proliferated, as shown in FIGS. 5C and 5E. The activated HFSCs retained BrdU for at least 3 days, as shown in FIG. 5E, while the transient amplifying stem cells lost the BrdU label within that 3 day period (data not shown). This indicated that transient amplifying stem cells proliferated more rapidly than activated HFSCs. Only a few cells in the mutant cyst boundary were BrdU-labeled when analyzed at three hours, as shown in FIG. 5D. Transient amplifying stem cells were characterized as three hour post-BrdU pulse labeled cells, which were located in the bulge and the bottom region of the hair bulb (HB) in Wt HFs, as shown in FIG. 5C. These transient amplifying HFSCs were also found in the base of the “bulb”-like structures of tumorous HFs, as shown in FIG. 5D.

Whether migratory patterns of activated stem cell population in Bmpr1a mutant HFs was similar to that of Wt HFs was investigated. It was found that the activated stem cell population increased in number over Wt in Bmpr1a mutant HFs, as measured by the presence of elevated 3 day post-labeled BrdU cells, as shown in FIG. 5F, as compared to FIG. 5E. As such, more BrdU-labeled cells were detected in the bulge-like area of mutant HFs, along the path migrating down to each “bulb”-like structure, and in the cyst boundary. These three day post-labeled activated stem cells, shown in FIG. 5F, were observed migrating to the “bulb”-like structures in cysts of the Bmpr1a mutant, as assessed by examination of multiple HF sections. Significantly, the activated HFSCs in mutant mice were enriched in the center of the early stage of the tumorous HFs, which eventually transformed into the cyst structure observed in matricomass.

Activated HFSCs, or three day post-BrdU pulse labeled cells, were observed to be located in the Wt bulge, as shown in FIG. 5E. These post-BrdU pulse labeled cells were characterized as migrating cells which traveled to the hair matrix (HM) in the Wt control, as shown in FIG. 5E. Thus, it appeared that the normal Wt activated HFSC migratory pattern from bulge to hair matrix was similar to the aberrant mutant HFSC migratory pattern, where 3 day post-labeled activated HFSCs migrated from the bulge-like area to each “bulb”-like structure and to the cyst boundary.

Cyst boundary cells also generally retained BrdU label for 3 days, and traveled on migration pathways, similar to Wt activated HFSCs, as shown in FIG. 5F. Once activated, the HFSCs began S to divide and migrate down to the base of the hair matrix (HM) in Wt mice, where these HFSCs became transient amplifying (TA) stem cells. Cycling transient amplifying HFSCs were characterized by three hour post-BrdU pulse-labeled cells. Similar to the migratory pattern observed in Wt mice, HFSCs migrated to “bulb”-like structures in Bmpr1a mutants.

Transient amplifying stem cells are Ki67-positive (i.e., proliferating cells). As shown in FIG. 5G, proliferating cells were detected in the bottom region of each Wt hair bulb structure, specifically the hair matrix and dermal papilla. Transient amplifying stem cells were characterized as three hour post-BrdU pulse labeled cells, which were located in the bulge and the bottom region of the hair bulb (HB) in Wt HFs, as shown in FIG. 5C. These transient amplifying HFSCs were also found in the base of the “bulb”-like structures of tumorous HFs, as shown in FIG. 5D. The number of proliferating Ki67 positive HFSCs was also increased in mutants over Wt, as shown in FIG. 5H. The cyst indicated a significant population of proliferating cells. The Ki67 proliferation marker, highly expressed in rapid cycling transient amplifying HFSCs, was detected both in hair matrix cells of Wt HFs, and in the bottom region of “bulb”-like structures of tumorous mutant HFs.

Activated HFSCs and transient amplifying HFSCs were also distinguishable by their Ki67 staining properties, where Ki67 served as a proliferation marker. HFSCs in the activated stage were weakly stained with Ki67, as shown in FIG. 5G. This weak staining verified that activated HFSCs were 3 day BrdU cells. However, the more rapidly cycling transient amplifying HFSC population was strongly stained with Ki67. This strong Ki67 staining was consistent with loss of BrdU over 3 days as discussed previously. Ki67, as an indicator of the presence of dividing transient amplifying HFSCs, was detected in the hair matrix cells of Wt HFs, as shown in FIG. 5G. Ki67 was also found in the bottom region of each “bulb”-like structure of tumorous HFs, as shown in FIG. 5H, indicating these aberrant tumor cells were also proliferating. Like activated HFSCs of Wt tissue, cyst boundary cells were Ki67-negative (i.e., nonproliferating cells) as shown in FIG. 5H.

To confirm the migration patterns of stem cells in the Wt and mutant HF regions as described above, the expression distribution of β1-integrin was analyzed. β1-integrin is a HF stem cell marker for a receptor that mediates HF cell-cell interactions. β1-integrin was detected in both the bulge and bottom of the hair matrix (HM) areas in normal Wt HFs, as shown in FIG. 5I. In the tumorous HF, β1-integrin was detected in the cyst boundary and bottom of the “bulb”-like structure, as shown in FIG. 5J. These observations clearly indicated that the dynamic process of cell migration in the tumorous HF still resembled that observed in the normal HF. Taken together with previous results, β1-integrin results confirmed that the bottoms of both normal Wt hair matrix and abnormal “bulb”-like structures in mutants were enriched with transient amplifying (TA) stem cells, while the cyst boundary in HF tumors was enriched with activated HFSCs. β1-integrin was found in Wt HFs in the bulge and bottom region of the hair bulb, in mutant “bulb”-like structures and tumorous HF cyst boundaries. Results obtained confirmed that hair follicle cell migration patterns were similar in both normal and mutant tumor tissue.

Cumulatively taken together with results from previous examples, it was determined that Wt HFSCs progressed through three distinct stages: (1) arrested stage, (2) activated stage, and (3) transient amplifying (TA) stage, as defined by their cell cycle state. The Wt HFSCs located in the bulge area were usually quiescent or arrested, but HFSCs became activated in response to initiation signals in early anagen. Significantly, blockage of the BMP signal to the receptor in the Bmpr1a mutant mouse resulted in expansion in the HFSC population number as characterized by increased expression of BrdU, Ki67, and β1-integrin proliferation markers.

### Example 17

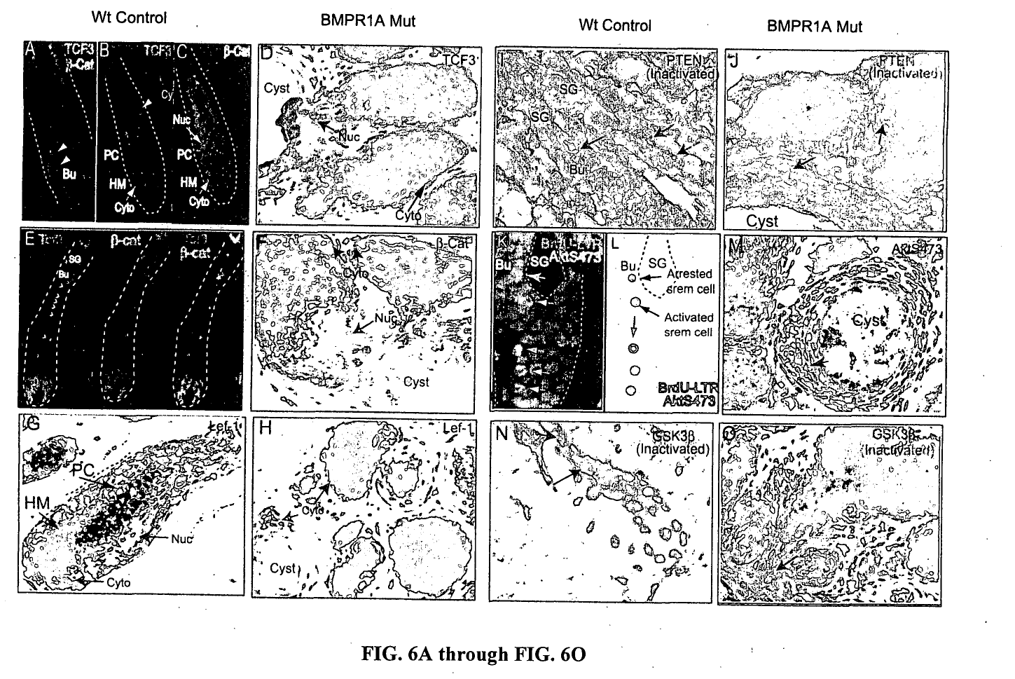

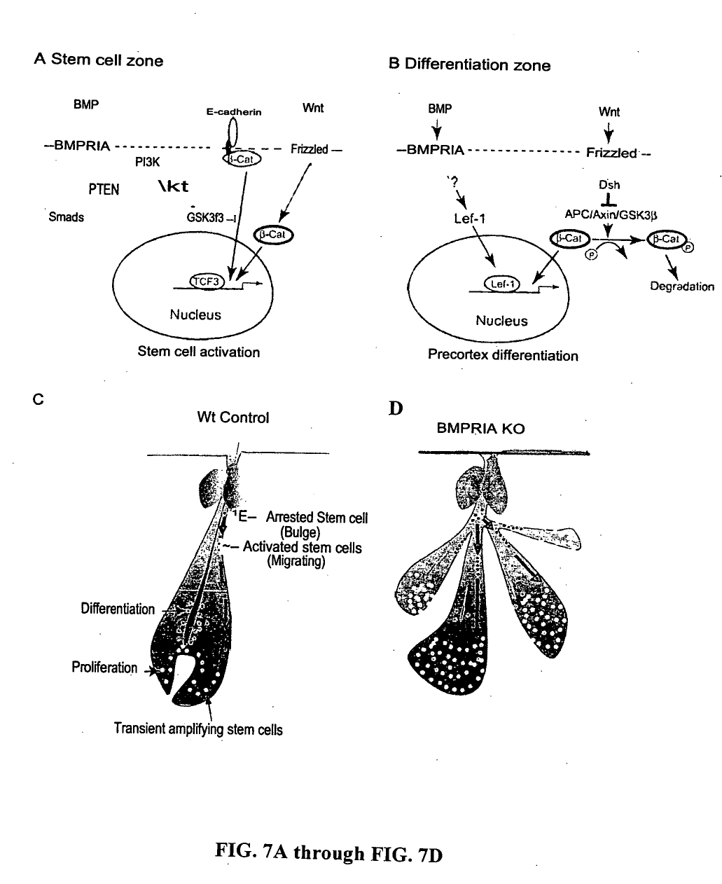

The presence of Wnt and BMP biochemical pathway markers was examined in HF tissue of Bmpr1a mutant mice and Wt control mice, to characterize activation and differentiation events in normal and aberrant mutant HFSC populations. The BMP pathway components include BMP, Bmpr1a, Smad, PTEN, PI3K, Akt, GSK3β, Tcf3, and Lef-1, as depicted in FIGS. 7A and 7B. The Wnt pathway includes Frizzled, Dsh, APC/Axin/GSK3β, and β-catenin. Distribution patterns for selected Wnt and BMP pathway components, specifically β-catenin, Tcf3, Lef-1, GSK3β, Akt, and PTEN markers, were examined in hair follicle tissue sections of Wt and mutant Bmpr1a mice using immunohistological staining methods, as shown in FIG. 6. BrdU co-staining was also examined. The Wt HF tissue marker distribution pattern depicted that the HF cell normally underwent progression from arrested HFSC to activated HFSC, migratory transient amplifying HFSC, and differentiating precortex cell stages. In contrast, in Bmpr1a mutant HF tissue, marker distributions revealed stunted maturation in tumors, characterized by the presence of activated HFSCs in the cyst boundaries, and an absence of markers associated with transient amplifying or differentiated precortex cells in tumorous regions.

In the normal Wt hair follicle (HF), cellular localization patterns for Tcf3, β-catenin, and Lef-1 dynamically changed in HF stem cell transitional stages from arrested through activation, proliferation, and differentiation stages. Arrested HFSCs in the bulge were characterized by nuclear localization of Tcf3, as shown in FIG. 6A. In contrast, Tcf3 was mainly localized in the cytoplasm of the Wt hair matrix (HM) cells, as shown in FIG. 6B, although Tcf3 was also expressed in other regions of the HFs. The Tcf3 distribution pattern in Wt HFs indicated that nuclear to cytoplasmic translocations of Tcf3 were associated with changes in HF stage from the arrested stage in the bulge (nuclear) to the activated and differentiated stages in the hair matrix (cytoplasm).

In the mutant HF, Tcf3 signals were detected in the nuclei of the cyst boundary cells, and the cytoplasm of cells in the “bulb”-like structures of tumorous HFs, as shown in FIG. 6D. In tumorous HFs, the presence of Tcf in nuclei of cyst boundary cells and cytoplasm of “bulb”-like structures, indicates potential existence of cells in mutant tissue bearing markers and phenotypic structures similar to those of Wt tissue-related arrested HFSCs and HM cells, respectively.

The presence of Tcf3 in the nuclei of arrested Wt HFSCs also indicated potential interaction with another stem cell regulator, β-catenin. β-catenin was studied to determine its presence in the nucleus of HFSCs. Results from costaining experiments revealed that β-catenin, which usually associates with E-cadherin in the membrane of the arrested stem cell, played a role in stem cell activation in the normal HF by translocating into the nucleus and forming a complex with Tcf3, as shown in FIG. 6A. β-catenin signals were detected in the following Wt HF tissues: membranes of HFSCs in the bulge, as depicted in FIGS. 6A and 6C; cytoplasm of the hair matrix (HM) cells, as shown in FIG. 6C; and nuclei of precortex cells, as shown in FIG. 6C. In mutant HF tissue, β-catenin was found in cytoplasm of multiple HF cells, nuclei of cells located in the cyst boundary, and cells migrating along the side of each “bulb”-like structure in tumorous HFs, as shown in FIG. 6F.

While analyses of individual β-catenin staining patterns provided useful data, it was necessary to analyze simultaneous Tcf3 and β-catenin co-staining distributions to determine potential interactions between these two regulatory molecules. As such, co-localization of β-catenin and Tcf3 was detected in the nuclei of downward-migrating, activated Wt HFSCs, as shown in FIGS. 6A and 6E. The HFSC migratory pattern is depicted by cells located at the white arrows in the left panel of FIG. 6E. When the activated Wt stem cell reached the bottom of the hair matrix (HM) region, it metamorphosized into a transient amplifying stem cell, as shown in FIG. 6E. At that time, the Tcf3 and β-catenin molecules translocated into the cytoplasm, as shown in FIGS. 6B and 6C.

In Bmpr1a mutant HF tumors, however, both Tcf3 and β-catenin were localized in the nuclei of the majority of cyst boundary cells, as shown in FIGS. 6D and 6F. This finding reinforced the deduction that these cells were identifiable activated HFSCs, as shown in the Wt HF photo of FIG. 6E. Thus, the population of the activated HFSCs was significantly expanded in tumors in comparison to Wt. Activated HFSCs in tumors indicated that Tcf3 and β-catenin in the nuclei of HFSCs induced increased stem cell activation, as diagramatically illustrated in FIG. 7A.

Both β-catenin and Lef-1 signals were required for Wt differentiation of precortex cells to hair shaft cells, and both of these markers co-existed simultaneously in the nuclei of precortex cells in normal HFs, as shown in FIGS. 6C and 6G. Lef-1 signals were detected in the nuclei of precortex cells in the Wt HFs, as shown in FIG. 6G, and in the cytoplasm of cells of the “bulb”-like structures of tumorous HFs, as shown in FIG. 6H. However, neither Lef-1 nor β-catenin was detected in the nuclei of cells in the tumorous “bulb”-like structures, as shown in FIGS. 6H and 6F, respectively. The switch from nuclear to cytoplasmic appearance of β-catenin and Lef-1 in mutant as compared to Wt tissue indicated that these regulatory molecules inhibited hair shaft differentiation in tumorous tissue. Aberrant HFSC expansion in Bmpr1a mutant tumors was consistent with this view.

Nuclear co-localization of β-catenin and Lef-1 coincided with nuclear localization of Smad4 in precortex cells of the normal HF (data not shown). This co-localization indicated mechanistic coordination between the BMP and Wnt signaling pathways for commitment to hair shaft lineage differentiation, as illustrated diagramatically in FIG. 7B. In accordance with previous observations, the BMP4 signal appeared required for expression of Lef-1. Furthermore, in the Bmpr1a mutant HF, the lack of nuclear localization of β-catenin and Lef-1, together with the loss of nuclear localization of Smad4, resulted in impaired differentiation of hair shaft cells. This finding further supported the synergistic roles of BMP and Wnt signals in commitment of these cells to hair shaft lineage.

Mechanistically, results supported the concept that interactions between the BMP pathway signals and the β-catenin/Wnt pathway signals occurred in discrete stem cell activation/proliferation zones and differentiation zones. Blocking the BMP pathway in the stem cell zone of Bmpr1a knockout mutants resulted in unchecked activation and proliferation of HFSCs, leading to continuously generated dysplastic HF structures, as illustrated diagrammatically in FIG. 7C. Within each abnormal “bulb”-like pre-neoplastic structure, as shown in FIG. 7C, right panel, hair shaft differentiation was impaired. Thus, disruption of BMP-Wnt pathway signals in the mutant mice led to aberrant hair follicle structures and tumorigenesis.

In summary, cellular localization patterns for Tcf3, β-catenin, and Lef-1 in normal HF tissue changed dynamically during transitions from stem cell activation to proliferation and differentiation. Marker studies established that the activated Wt HFSC migrated to the bottom of the hair matrix region, then became transformed into a transient amplifying stem cell. Subsequently, the Tcf3 and β-catenin molecules translocated into the cytoplasm of the Wt cell. The nuclear co-existence of both β-catenin and Lef-1 signals was required for normal differentiation of precortex cells to hair shaft cells. In contrast, in Bmpr1a mutant HF tumors, Tcf3 and β-catenin markers co-existed only in the nuclei of activated HFSCs in the cyst boundary, with impaired hair shaft differentiation leading to HF tumorigenesis.

### Example 18

Since previous examples documented that BMP signaling was important in HFSC activation and proliferation, the presence of downstream pathway components of BMP signaling was examined in activated HF cells. These downstream components included PTEN, Akt, and GSK-3 β.

BMP signaling downstream pathway components in hair follicle tissue were characterized by immunofluorescent and immunohistological methods. Antibodies specific for PTEN (26H9), phospho-PTEN (Ser/380/Thr382/383), Akt (5G3), phospho-Akt (Ser473), GSK-3 β, and phospho-GSK-3 β (Ser9) were obtained (Cell Signaling Technology, Inc., Beverly, Mass.). The secondary antibody was detected with AEC (red) or by fluorescence, and the cells were counter-stained with hematoxylin or DAPI (blue). Accordingly, immunofluorescent staining was performed by incubating with fluorophore-conjugated secondary antibodies (Molecular Probes, Invitrogen, Carlsbad, Calif.) after primary antibody incubation, and mounted with DAPI blue fluorescent counterstain (InnoGenex, CS-2010-06) or hematoxylin. Images were visualized by fluorescence microscopy (Zeiss, VWR Scientific, Swanee, Ga.).

Significantly, as predicted, activated Wt HFSCs contained the following three BMP-downstream markers: the inactivated form of PTEN, the activated form of Akt (Akt-S473), and the inactivated form of GSK3β (GSK3β-S9), as shown in FIGS. 6I, 6K, and 6N, respectively. Inactivated PTEN-P3 was detected in the activated HFSCs situated in the Wt bulge region and in downward-migrating stem cells as shown in FIG. 6I. Inactivated PTEN-P3 was also found in the cyst boundary cells, as shown in FIG. 6J.

Activated Wt HFSCs also exhibited detectable Akt-S473 red staining as these cells were beginning their migration from the bulge, as shown at the white arrows of FIG. 6K. In contrast, the arrested HFSC population was positive for BrdU-LTR, but not for Akt-S473, as shown by the green-stained cell at the top white arrow of FIG. 6K. As the arrested HFSCs transformed into activated HFSCs that divided and migrated downward, the BrdU-LTR staining intensity faded, as shown in FIG. 6K, and diagramatically shown in FIG. 6L.

The view was presented that Akt activation was involved in translocation of β-catenin from the cytoplasmic, E-cadherin-associated complex, into the nucleus through suppression of GSK3β activity, as diagrammatically illustrated in FIG. 7A. Moreover, Akt potentially appeared to provide a temporal protection for the activated stem cells since Akt possessed the ability to prevent cells from anoikis, a form of apoptosis initiated when cells detach from the extracellular matrix (data not shown).

In Bmpr1a mutant hair follicles, activated HFSCs, containing the triad of inactivated PTEN, activated Akt, and inactivated GSK3β, were located in the cyst boundary and in downward-migrating stem cells, as shown in FIGS. 6J, 6M, and 6O, respectively. These two cell types also exhibited nuclear-localization of β-catenin in mutants, as shown in FIG. 6F. The GSK3β-S9 marker exhibited a similar distribution pattern as that of PTEN-P3 in Wt and mutant mice, as shown in FIGS. 6N and 6O, respectively. These results indicated that the PI3K-Akt pathway was involved in the control of GSK3β activity in the arrested stem cells. Moreover, this GSK3β control was suppressed by BMP signaling through PTEN.

Cumulatively, results in the Wt stem cell zone, depicted that release of BMP binding to Bmpr1a receptor resulted in triggering of the PTEN/PI3K/Akt/GSK3 (biochemical cascade, as diagramatically illustrated in FIG. 7A. Activated HFSCs of early anagen possessed inactivated PTEN, activated Akt-S473, and inactivated GSK3 (Activated HFSCs exhibit BrdU-LTR staining properties, but staining intensity faded as these cells divided and migrated downward from the bulge towards the hair matrix (HM). Proliferation of activated HFSCs was preceded by β-catenin translocation into the nucleus where β-catenin/Tcf3 interaction occurred. The normal progression from arrested HFSCs in the bulge to activated HFSCs migrating down the Wt hair shaft to differentiating or proliferating HFSCs is diagrammatically presented in FIG. 7C. In the differentiation zone, located between the bulge and hair matrix regions, BMP and Wnt pathways interacted through Lef-1 and β-catenin molecules, respectively, as these two molecules translocated into the nucleus, as illustrated in FIG. 7B.

As in the Wt hair follicle, blocking the BMP signal in the Bmpr1a mutant hair follicles resulted in activation of the HFSC population through triggering of a cascade of PTEN-Akt-GSK3β interactions, ultimately leading to translocation of β-catenin into the nucleus. Blocking the BMP signal, indicated by loss of Smad4 nuclear-localization in activated stem cells, as shown in FIGS. 1F-1H, generated a cascade of PTEN-Akt-GSK3β activity. This cascade ultimately led to translocation of β-catenin into the nucleus, as shown in FIG. 6C, and as shown diagrammatically in FIG. 7A. Significantly, BMP pathway disruption also resulted in the dysplastic formation of HF tumors in the Bmpr1a mutant knockout animals, as illustrated diagramatically in FIG. 7D. Tumors were caused by aberrant BMP regulation in mutants, leading to nuclear localization of -catenin and resulting in significantly greater HFSC population proliferation and expansion over Wt.

### Example 19

Immunohistological staining methods utilizing antibodies directed against differentiation markers was performed on HF tissue derived from Wt and Bmpr1a mutant mice to obtain staining results described in subsequent examples. The procedure involved fixing mouse skin in formalin, blocking endogenous biotin, adding primary antibodies directed against differentiation antigens, incubating with secondary enzyme-labeled antibodies, and adding substrate to visualize the differential staining pattern characterizing hair follicle structural features.

First, mouse skin was collected and fixed overnight in zinc formalin (Richard-Allan Scientific, Wayne, N.J.) at room temperature, dehydrated, embedded in wax, and sectioned. After deparaffinization following standard procedures, epitope unmasking was accomplished using 10 mM citrate buffer (pH 6.0) in an electric pressure cooker (Biocare Medical, Walnut Creek, Calif.) at 120° C., 15 psi. The sections were rinsed with distilled water, followed by 3% hydrogen peroxide treatment at room temperature (RT) to block endogenous peroxidase.

Second, endogenous biotin was blocked on the mouse skin tissue, when applicable, using the Avidin/Biotin block kit (Vector Laboratories, Inc., Burlingame, Calif.). Non-specific antibody binding was blocked using a combination of 2% normal mouse serum and 10% normal goat serum in PBS for 30 minutes.

Third, the primary antibodies directed against hair follicle antigens were added to the avidin/biutin blocked mouse skin. These primary antibodies were as follows: mouse AE13 and AE15 monoclonal antibodies (gifts from Dr. T. T. Sun); mouse monoclonal antiCK-14 (Novocastra Laboratories Ltd., distributed by Vector Labs., Burlingame, Calif., NCL-LL002); monoclonal anti-CK10 (Sigma-Aldrich, St. Louis, Mo.); anti-CK5 serum (Covance Laboratories, Harowgate, UK LN#14430002); anti-β-catenin serum (Sigma, St. Louis, Mo., Product No. C2206); anti-β-catenin monoclonal antibody (8E6, anti-active-catenin, anti-ABC, Upstate Biotechnology, Lake Placid, N.Y.), goat anti-β-catenin serum (Santa Cruz Biotechnology, Inc., Santa Cruz, Calif.), anti-Tcf3 and anti-Lef1 monoclonal antibodies (Upstate Biotechnology, Lake Placid, N.Y., Cat. No. 05-512 and 05-602); anti-integrin β1 serum (Santa Cruz Biotechnology, Inc., Santa Cruz, Calif., #sc-8978); anti-Ki67 serum (DAKO, Carpenteria, Calif., #M7249); and anti-Bmpr1a serum (gift from Dr. P. Dijke, Netherlands), PTEN monoclonal antibody (26H9, Cell Signaling Technology, Beverly, Mass.), Cyclin D1 monoclonal antibody (DCS6, Cell Signaling Technology), rabbit anti-isll polyclonal antibody (Chemicon International, Inc., Temecula, Calif.), monoclonal anti-E-cadherin monoclonal antibody (Zymed Laboratories, Inc., South San FrancHFSCo, CA), P63 (Biocare Medical), anti-Phospho-PTEN (Cell Signaling Technology), anti-Phospho-AKT antibody (Ser473, Cell Signaling Technology), and anti-AKT antibody (Cell Signaling Technology). For mouse primary monoclonal antibodies, the DAKO ARK™ kit (DAKO, Carpinteria, Calif., #K3954) was used for biotinylation of the antibodies and subsequent streptavidin-HRP incubation.

Fourth, the secondary antibody against the primary antibodies (e.g., AE 13, 15, CK5, 10, 14) was detected by AEC staining (red) with immunoperoxidase enzyme label. For secondary anti-rabbit antiserum, DAKO Envision™+labeled polymer horseradish peroxidase (HRP) was used with HRP-anti-rabbit antiserum (DAKO, Carpenteria, Calif.). For rat anti-mouse serum, biotin-conjugated goat anti-rat secondary antibody was used followed by streptavidin-HRP incubation.

Finally, localization of the HRP enzyme activity in hair follicle tissue sections was identified using amino-ethyl carbozole substrate (AEC+substrate-Chromogen, DAKO, Carpenteria, Calif.; Sigma-Aldrich, St. Louis, Mo.—20 mg AEC tablets). The nuclear counterstain was employed hematoxylin (blue). AEC Substrate Stock Solution and AEC Working Solution were made according to Sigma protocols. Standard trichrome staining was performed using the Sigma manufacturer's procedure (#HT15, Sigma, St. Louis, Mo.).

### Example 20

BrdU pulse and/or long term labeling provided a method for determining whether HFSCs at an identified location in the hair follicle existed in an arrested or proliferating stage. Arrested HFSCs generally retained the pulse-labeled BrdU for a relatively long time (>2 months for BrdU-LTR cells), whereas proliferating HFSCs (activated and transient amplifying) typically lost their DNA-containing BrdU label gradually over time. As such, characterization of the BrdU labeling properties of cells permitted distinguishing between the first arrested stage and the subsequent activated and transient amplifying (TA) stages of HFSCs.

The BrdU Tissue Staining Kit (Zymed Laboratories, Inc., South San Francisco, Calif.), used for staining studies, contained a biotinylated anti-BrdU antibody used in conjunction with streptavidin-peroxidase enzyme. Nuclei, incorporating BrdU label, bound the specific antibody and peroxidase conjugate; and nuclei were then stained (dark brown) by the diaminobenzidine (DAB) chromogen, set against a hematoxylin (blue) counterstain.

To characterize hair follicle stem cells (HFSCs) for the presence of long-term retention of the BrdU label (BrdU-LTR cells), pre-excision Mx1-Cre-Lox Bmpr1a mutant pups were subcutaneously injected with BrdU (10 mg/kg body weight) twice a day for 7 days starting from the first day after birth. P2-induced pups were injected with PolyI:C, as described previously, to induce Cre-mediated DNA recombination. Skin was collected on day 70 after BrdU labeling. To identify the proliferating cells by BrdU pulse labeling, skin was collected 3 hours after a single intraperitoneal (I.P.) injection of BrdU (1 mg/mouse). Skin was processed as described above and sectioned. BrdU in situ staining was performed using the Zymed BrdU staining kit.

### Example 21

Murine stem cell virus (MSCV) (University of Missouri Animal Diagnostic Research Laboratory, Columbia, Mo.) can be utilized to transduce genes into recipient hematopoietic cells (HSCs) to induce self-renewal and proliferation in an HSC population in vitro. Transfection of plasmids containing these genes into HSC populations is followed by in vitro cultivation of HSC populations. Alternatively, MSCV transduction of HSC populations can be performed.

The dominant negative mutant (DN) form of PTEN (DN-PTEN) and activated Akt genes can be fused to an estrogene regulatory (ER) DNA fragment and subcloned into the MSCV vector. After subcloning, the MSCV-ER-PTEN and MSCV-ER-DN-activated Akt plasmids are transfected into separate HSC populations; and the transfected HSC cells are selected based on GFP expression, which is driven by an internal ribosomal entry site (IRES) sequence in a bicistronic vector. The GFP positive HSC cells are induced with an ER inducer, such as tamorxiphan, wherein the inserted DN-PTEN or activated Akt genes can be activated such that the HSCs undergo proliferation and self-renewal. After in vitro culture of the HSC population for 20 generations, HSC cells are tested for cell replication rate; telomere length; the presence of Lin−, Sca−1+and Kit+markers; and the nucleus to cytoplasm ratio. In addition, HSCs are transplanted into recipient mice to test whether donor stem cells can reconstitute differentiated lymphocyte and myeloid cell lineages of the hematopoietic system.

The method for tranducing murine HSCs with MSCV virus used is as follows: Murine BM HSCs are mobilized using G-CSF for 2-3 days prior to harvesting bone marrow. Isolated murine lineage-depleted (Lin-negative) cells were obtained by fluorescence cell sorting by standard techniques. See Park, I-K, et al. (2002). HSCs were cultured with 2 ml of QBSF medium with TSF (Tpo=10 ng/ml; SCF=100 ng/ml, FL=100 ng/ml) in a 6 well culture plate. A cell density of 10⁶ cells/ml was preferred. The next morning, 2 ml of Lin-negative cells can be centrifuged (500×g) and the supernatant decanted. 2 ml of Virals Supernatantor, containing the MSCV-ER-PTEN and MSCV-ER-activated Akt plasmids, and 2 ul Polybrene (8 μg/ml final concentration) can be added to the HSC cells, centrifuged, washed in QBSF medium with or without TSF, and incubated at 37° C. for three days. It generally takes 3-4 cell divisions to reach maximal transgene expression of the transfected HSC cells. The transfected HSCs are selected based on GFP expression and HSC markers (Lin-negative Sca−1+c−Kit+) by FACS cell sorting, which is driven by IRES (Internal Ribosome Entry Site) sequence.

The above methods should result in the following DN-PTEN-induced and activated Akt-induced activities in the HSC cell population: maintenance of telomerase activity important in increasing replication potential; inhibition of P53 activity, essential for overcoming cell senescence; increase in cell survival through suppression of cell apoptotic signals; increased proliferation and self-renewal functions resulting in expansion of the HSC population; and maintenance of the multipotential ability of the HSC population to differentiate into myeloid and lymphoid lineage cells upon transplantation into a severe combined immunodeficiency (SCID) mouse repopulating assay system.

Thus, compositions and methods have been shown for controlling hair follicle stem cell fate. It is apparent to those skilled in the art, however, that many changes, variations, modifications, and other uses and applications to the composition and method related to controlling stem cell fate are possible, and also such changes, variations, modifications, and other uses and applications that do not depart from the spirit and scope of the invention are deemed to be covered by the invention, which is limited only by the claims which follow.

### REFERENCES

Blessing, M., Nanney, L. B., King, L. E., Jones, C. M., and Hogan, B. L. (1993). Transgenic mice as a model to study the role of TGF-beta-related molecules in hair follicles. Genes Dev 7, 204-215.
Botchkarev, V. A. (2003). Bone morphogenetic proteins and their antagonists in skin and hair follicle biology. J Invest Dermatol 120, 36-47.
Botchkarev, V. A., Botchkareva, N. V., Nakamura, M., Huber, O., Funa, K., Lauster, R., Paus, R., and Gilchrest, B. A. (2001). Noggin is required for induction of the hair follicle growth phase in postnatal skin. FASEB J 15, 2205-2214.
Botchkarev, V. A., Botchkareva, N. V., Roth, W., Nakamura, M., Chen, L. H., Herzog, W., Lindner, G., McMahon, J. A., Peters, C., Lauster, R., et al. (1999). Noggin is a mesenchymally derived stimulator of hair-follicle induction. Nat Cell Biol 1, 158-164.
Bunting, K. D. Ann. N.Y. Acad. Sci. 125-141 (1999).
Cotsarelis, G., Sun, T. T., and Lavker, R. M. (1990). Label-retaining cells reside in the bulge area of pilosebaceous unit: implications for follicular stem cells, hair cycle, and skin carcinogenesis. Cell 61, 1329-1337.
Cross, D. A., Alessi, D. R., Cohen, P., Andjelkovich, M., and Hemmings, B. A. (1995). Inhibition of glycogen synthase kinase-3 by insulin mediated by protein kinase B. Nature 378, 785-789.
Dale, L., and Wardle, F. C. (1999). A gradient of BMP activity specifies dorsal-ventral fates in early Xenopus embryos. Semin Cell Dev Biol 10, 319-326.
DasGupta, R., and Fuchs, E. (1999). Multiple roles for activated LEF/TCF transcription complexes during hair follicle development and differentiation. Development 126, 4557-4568.
Ema, H. et al. J. Exp. Med. 192: 1281-1288 (2000).
Eng, C. (2001). To be or not to BMP. Nat Genet 28, 105-107.
Entchev, E. V., Schwabedissen, A., and Gonzalez-Gaitan, M. (2000). Gradient formation of the TGF-beta homolog Dpp. Cell 103, 981-991.
Fuchs, E., and Segre, J. A. (2000). Stem cells: a new lease on life. Cell 100, 143-155.
Fukumoto, S., Hsieh, C. M., Maemura, K., Layne, M. D., Yet, S. F., Lee, K. H., Matsui, T., Rosenzweig, A., Taylor, W. G., Rubin, J. S., et al. (2001). Akt participation in the Wnt signaling pathway through Dishevelled. J Biol Chem 276, 17479-17483.
Gallego, M. I., Beachy, P. A., Hennighausen, L., and Robinson, G. W. (2002). Differential requirements for shh in mammary tissue and hair follicle morphogenesis. Dev Biol 249, 131-139.
Gat, U., DasGupta, R., Degenstein, L., and Fuchs, E. (1998). De novo hair follicle morphogenesis and hair tumors in mice expressing a truncated beta-catenin in skin. Cell 95, 605-614.
Graff, J. M. (1997). Embryonic patterning: to BMP or not to BMP, that is the question. Cell 89, 171-174.
Groszer, M., Erickson, R., Scripture-Adams, D. D., Lesche, R., Trumpp, A., Zack, J. A., Komblum, H. I., Liu, X., and Wu, H. (2001). Negative regulation of neural stem/progenitor cell proliferation by the Pten tumor suppressor gene in vivo. Science 294, 2186-2189.
Hardy, M. H. (1992). The secret life of the hair follicle. Trends Genet 8, 55-61.
Headon, D. J., and Overbeek, P. A. (1999). Involvement of a novel Tnf receptor homologue in hair follicle induction. Nat Genet 22, 370-374.
Hogan, B. L. (1996). Bone morphogenetic proteins: multifunctional regulators of vertebrate development. Genes Dev 10, 1580-1594.
Huelsken, J., Vogel, R., Erdmann, B., Cotsarelis, G., and Birchmeier, W. (2001). beta-Catenin controls hair follicle morphogenesis and stem cell differentiation in the skin. Cell 105, 533-545.
Jones, P. H., Harper, S., and Watt, F. M. (1995). Stem cell patterning and fate in human epidermis. Cell 80, 83-93.
Khwaja, A., Rodriguez-Viciana, P., Wennstrom, S., Warne, P. H., and Downward, J. (1997). Matrix adhesion and Ras transformation both activate a phosphoinositide 3-OH kinase and protein kinase B/Akt cellular survival pathway. EMBO J 16, 2783-2793.
Kimura, T., Suzuki, A., Fujita, Y., Yomogida, K., Lomeli, H., Asada, N., Ikeuchi, M., Nagy, A., Mark, T. W., and Kakano, T. (2003). Conditional loss of PTEN leads to testicular teratoma and enhances embryonic germ cell production. Development 130, 1691-1700.
Kishimoto, J., Burgeson, R. E., and Morgan, B. A. (2000). Wnt signaling maintains the hair-inducing activity of the dermal papilla. Genes Dev 14, 1181-1185.
Kopan, R., Lee, J., Lin, M. H., Syder, A. J., Kesterson, J., Crutchfield, N., Li, C. R., Wu, W., Books, J., and Gordon, J. I. (2002). Genetic mosaic analysis indicates that the bulb region of coat hair follicles contains a resident population of several active multipotent epithelial lineage progenitors. Dev Biol 242, 44-57.
Kratochwil, K., Dull, M., Farinas, I., Galceran, J., and Grosschedl, R. (1996). Lef1 expression is activated by BMP-4 and regulates inductive tissue interactions in tooth and hair development. Genes Dev 10, 1382-1394.
Kuhn, R., Schwenk, F., Aguet, M., and Rajewsky, K. (1995). Inducible gene targeting in mice. Science 269, 1427-1429.
Kulessa, H., and Hogan, B. L. (2002). Generation of a LoxP flanked bmp4LoxP-lacZ allele marked by conditional lacZ expression. Genesis 32, 66-68.
Kulessa, H., Turk, G., and Hogan, B. L. (2000). Inhibition of Bmp signaling affects growth and differentiation in the anagen hair follicle. EMBO J 19, 6664-6674.
Larochelle, A. et al. Nat. Med 2: 1329-1337 (1996).
Lavker, R. M., Miller, S., Wilson, C., Cotsarelis, G., Wei, Z. G., Yang, J. S., and Sun, T. T. (1993). Hair follicle stem cells: their location, role in hair cycle, and involvement in skin tumor formation. J Invest Dermatol 101, 16S-26S.
Lim, D. A., Tramontin, A. D., Trevejo, J. M., Herrera, D. G., Garcia-Verdugo, J. M., and Alvarez-Buylla, A. (2000). Noggin antagonizes BMP signaling to create a niche for adult neurogenesis. Neuron 28, 713-726.
Lin, H. (2002). The stem-cell niche theory: lessons from flies. Nat Rev Genet 3, 931-940.
Lin, M. H., Leimeister, C., Gessler, M., and Kopan, R. (2000). Activation of the Notch pathway in the hair cortex leads to aberrant differentiation of the adjacent hair-shaft layers. Development 127, 2421-2432.
Lynch, M. H., O'Guin, W. M., Hardy, C., Mak, L., and Sun, T. T. (1986). Acidic and basic hair/nail (“hard”) keratins: their colocalization in upper cortical and cuticle cells of the human hair follicle and their relationship to “soft” keratins. J Cell Biol 103, 2593-2606.
Maehama, T., and Dixon, J. E. (1998). The tumor suppressor, PTEN/MMAC1, dephosphorylates the lipid second messenger, phosphatidylinositol 3,4,5-trisphosphate. J Biol Chem 273, 13375-13378.
McMahon, J. A., Takada, S., Zimmerman, L. B., Fan, C. M., Harland, R. M., and McMahon, A. P. (1998). Noggin-mediated antagonism of BMP signaling is required for growth and patterning of the neural tube and somite. Genes Dev 12, 1438-1452.
Merrill, B. J., Gat, U., DasGupta, R., and Fuchs, E. (2001). Tcf3 and Lef1 regulate lineage differentiation of multipotent stem cells in skin. Genes Dev 15, 1688-1705.
Mishina, Y., Hanks, M. C., Miura, S., Tallquist, M. D., and Behringer, R. R. (2002). Generation of Bmpr/Alk3 conditional knockout mice. Genesis 32, 69-72.
Mishina, Y., Suzuki, A., Ueno, N., and Behringer, R. R. (1995). Bmpr encodes a type I bone morphogenetic protein receptor that is essential for gastrulation during mouse embryogenesis. Genes Dev 9, 3027-3037.
Murphy, G. F., and Mihm, J. M. C. (1999). Skin. In Robbins Pathological Basis of Diseases, R. Cotran, V. Kumar, and T. Collins, eds. (London, W. B. Saunders Company), pp. 1170-1213.
Nishimura, E. K., Jordan, S. A., Oshima, H., Yoshida, H., Osawa, M., Moriyama, M., Jackson, I. J., Barrandon, Y., Miyachi, Y., and Nishikawa, S. (2002). Dominant role of the niche in melanocyte stem-cell fate determination. Nature 416, 854-860.
Nolta, J. A. et al. Blood 83: 3041-3051 (1994).
Novak, A., Guo, C., Yang, W., Nagy, A., and Lobe, C. G. (2000). Z/EG, a double reporter mouse line that expresses enhanced green fluorescent protein upon Cre-mediated excision. Genesis 28, 147-155.
Nusse, R., Samos, C. H., Brink, M., Willert, K., Cadigan, K. M., Wodarz, A., Fish, M., and Rulifson, E. (1997). Cell culture and whole animal approaches to understanding signaling by Wnt proteins in Drosophila. Cold Spring Harb Symp Quant Biol 62, 185-190.
O'Guin, W. M., Sun, T. T., and Manabe, M. (1992). Interaction of trichohyalin with intermediate filaments: three immunologically defined stages of trichohyalin maturation. J Invest Dermatol 98, 24-32.
Park, I-K, et al. Blood 99(2): 488-498 (2002).
Persad, S., Troussard, A. A., McPhee, T. R., Mulholland, D. J., and Dedhar, S. (2001). Tumor suppressor PTEN inhibits nuclear accumulation of beta-catenin and T cell/lymphoid enhancer factor 1-mediated transcriptional activation. J Cell Biol 153, 1161-1174.
Povelones, M., and Nusse, R. (2002). Wnt signaling sees spots. Nat Cell Biol 4, E249-250.
Reya, T., Morrison, S. J., Clarke, M. F., and Weissman, I. L. (2001). Stem cells, cancer, and cancer stem cells. Nature 414, 105-111.
Schofield, R. (1978). The relationship between the spleen colony-forming cell and the hamatopopietic stem cell. A hypothesis. Blood Cells 4, 7-25.
Sinha, S., and Fuchs, E. (2001). Identification and dissection of an enhancer controlling epithelial gene expression in skin. Proc Natl Acad Sci USA 98, 2455-2460.
Smith, A., and Graham, A. (2001). Restricting Bmp-4 mediated apoptosis in hindbrain neural crest. Dev Dyn 220, 276-283.
Spradling, A., Drummond-Barbosa, D., and Kai, T. (2001). Stem cells find their niche. Nature 414, 98-104.
Stiles, B., Gilman, V., Khanrenzon, N., Lesche, R., Li, A., Qiao, R., Liu, X., and Wu, H. (2002). Essential role of AKT-1/proteinkinase B alpha in PTEN-controlled tumorigenesis. Mol Cell Biol 22, 3842-3851.
Sun, T. T., Cotsarelis, G., and Lavker, R. M. (1991). Hair follicular stem cells: the bulge-activation hypothesis. J Invest Dermatol 96, 77S-78S.
Taylor, G., Lehrer, M. S., Jensen, P. J., Sun, T. T., and Lavker, R. M. (2000). Involvement of follicular stem cells in forming not only the follicle but also the epidermis. Cell 102, 451-461.
Teleman, A. A., and Cohen, S. M. (2000). Dpp gradient formation in the Drosophila wing imaginal dHFSC. Cell 103, 971-980.
van Genderen, C., Okamura, R. M., Farinas, I., Quo, R. G., Parslow, T. G., Bruhn, L., and Grosschedl, R. (1994). Development of several organs that require inductive epithelial-mesenchymal interactions is impaired in LEF-1-deficient mice. Genes Dev 8, 2691-2703.
Vazquez, F., Ramaswamy, S., Nakamura, N., and Sellers, W. R. (2000). Phosphorylation of the PTEN tail regulates protein stability and function. Mol Cell Biol 20, 5010-5018.
Vooijs, M., Jonkers, J., and Bems, A. (2001). A highly efficient ligand-regulated Cre recombinase mouse line depicts that LoxP recombination is position dependent. EMBO Rep 2, 292-297.
Waite, L. A., and Eng, C. (2003). BMP2 exposure results in decreased PTEN protein degradation and increased PTEN levels. Hum Mol Genet 12, 679-684.
Watt, F. M., and Hogan, B. L. (2000). Out of Eden: stem cells and their niches. Science 287, 1427-1430.
Weedon, D., and Strutton, G. (1981). Apoptosis as the mechanism of the involution of hair follicles in catagen transformation. Acta Derm Venereol 61, 335-339.
Weissman, I. L. (1994). Developmental switches in the immune system. Cell 76, 207-218.
Widelitz, R. B., Jiang, T. X., Noveen, A., Chen, C. W., and Chuong, C. M. (1996). FGF induces new feather buds from developing avian skin. J Invest Dermatol 107, 797-803.
Yamamoto, O., Hamada, T., Doi, Y., Sasaguri, Y., and Hashimoto, H. (2002). Immunohistochemical and ultrastructural observations of desmoplastic trichoepithelioma with a special reference to a morphological comparison with normal apocrine acrosyringeum. J Cutan Pathol 29, 15-26.
Yi, S. E., Daluiski, A., Pederson, R., Rosen, V., and Lyons, K. M. (2000). The type I BMP receptor BMPR1B is required for chondrogenesis in the mouse limb. Development 127, 621-630.
Yokouchi, Y., Sakiyama, L., Kameda, T., Iba, H., Suzuki, A., Ueno, N., and Kuroiwa, A. (1996). BMP-2/-4 mediate programmed cell death in chicken limb buds. Development 122, 3725-3734.
Zhang, J., Tan, X., Contag, C. H., Lu, Y., Guo, D., Harris, S. E., and Feng, J. Q. (2002). Dissection of promoter control modules that direct Bmp4 expression in the epithelium-derived components of hair follicles. Biochem Biophys Res Commun 293, 1412-1419.
Zhou, P., Byrne, C., Jacobs, J., and Fuchs, E. (1995). Lymphoid enhancer factor 1 directs hair follicle patterning and epithelial cell fate. Genes Dev 9, 700-713.

What is claimed is:

1. A mutant HFSC comprising an inactive Bmpr1a receptor polypeptide, wherein BMP binding to Bmpr1a receptor polypeptide is substantially inhibited.

2. The mutant HFSC of claim 1, wherein the inactive Bmpr1a receptor polypeptide comprises a truncated Bmpr1a receptor polypeptide.

3. The mutant HFSC of claim 1, wherein the Bmpr1a receptor polypeptide is substantially inactivated.

4. The mutant HFSC of claim 2, wherein the truncated Bmpr1a receptor polypeptide is SEQ. ID. NO. 5:

5. A mutant HFSC comprising a mutant Bmpr1a nucleic acid sequence encoding an inactive Bmpr1a receptor polypeptide.

6. The mutant HFSC of claim 5, wherein the mutant Bmpr1a nucleic acid sequence is SEQ. ID. NO. 2.

7. The mutant HFSC of claim 5, wherein the mutant Bmpr1a nucleic acid sequence comprises a mutation selected from the group consisting of frame shift, substitution, loss of function, knockout deletion, and conventional deletion mutations.

8. The mutant HFSC of claim 5, wherein the HFSC is a mutant cell selected from the group consisting of mutant Bmpr1a transgenic organism cells, virus-transfected Bmpr1a-mutagenized organism cells, and in vitro Bmpr1a-mutagenized cells.

9. The mutant HFSC of claim 5, wherein the inactive Bmpr1a receptor is a truncated Bmpr1a receptor polypeptide sequence.

10. A HFSC comprising an isolated antibody selected from the group consisting of anti-Bmpr1a antibody, anti-BMP antibody, and fragments thereof, wherein the antibody induces HFSC proliferation in vitro by inhibiting BMP binding to Bmpr1a receptor.

11. A HFSC comprising an isolated in vitro Noggin activator selected from the group of activators consisting of Noggin polypeptide, and fragments thereof, wherein the Noggin activator induces HFSC proliferation in vitro by inhibiting BMP binding to Bmpr1a receptor.

12. A HFSC comprising an isolated Bmpr1a antisense fragment, wherein the antisense fragment induces HFSC proliferation by inhibiting translation of Bmpr1a receptor polypeptide.

13. A mutant Bmpr1a hair follicle cell comprising an inactivated Bmpr1a cell receptor polypeptide encoded by a mutant Bmpr1a gene selected from the group consisting of knockout deletion, conventional deletion, substitution, loss of function, and frame shift mutations.

14. The mutant Bmpr1a hair follicle cell of claim 13, wherein the inactivated Bmpr1a receptor polypeptide is located in a hair follicle cell selected from the group consisting of stem, resting, self-renewing, proliferating, transient amplifying, differentiating, apoptotic, and tumor cells.

15. A mutant Bmpr1a nucleic acid sequence of SEQ. ID. NO. 2, wherein the sequence encodes an inactive Bmpr1a receptor polypeptide.

16. A Bmpr1a nucleic acid sequence of SEQ. ID. NO. 3.

17. An isolated mutant Bmpr1a nucleic acid sequence encoding an inactive Bmpr1a receptor polypeptide.

18. The isolated mutant Bmpr1a nucleic acid sequence of claim 17, wherein the isolated mutant Bmpr1a nucleic acid sequence comprises a mutation selected from the group consisting of frame shift, substitution, loss of function, knockout deletion, and conventional deletion mutations.

19. A truncated inactive Bmpr1a receptor polypeptide of SEQ. ID. NO. 5, wherein amino acid numbers 34 to 77 are deleted from the wild type Bmpr1 receptor polypeptide.

20. A Bmpr1a polypeptide sequence of SEQ. ID. NO. 6.

21. An isolated inactive Bmpr1a receptor polypeptide, wherein Bmpr1a binding to BMP is substantially inhibited.

22. A vector comprising:
(a) a promoter; and,
(b) a stem cell activator selected from the group consisting of antisense Bmpr1a, DN-PTEN, activated Akt, Noggin, and activated PI3K.

23. A hair follicle stem cell comprising the vector of claim 22.

24. The vector of claim 22, wherein the vector is selected from the group consisting of expression vectors, fusion vectors, gene therapy vectors, two-hybrid vectors, reverse two-hybrid vectors, sequencing vectors, and cloning vectors.

25. The vector of claim 22, wherein the promoter is selected from the group consisting of a viral promoter and a cellular promoter.

26. The vector of claim 22, wherein the vector comprises a selectable marker selected from the group consisting of an antibiotic resistance gene, a tRNA gene, an auxotrophic gene, a toxic gene, a phenotypic marker, a colorimetric marker, an antisense oligonucleotide, a restriction endonuclease, an enzyme cleavage site, a protein binding site, and an immunoglobulin binding site.

27. The vector of claim 22, wherein the vector is selected from the group consisting of prokaryotic and eukaryotic vectors.

28. The vector of claim 26, wherein the selectable marker is selected from the group consisting of LacZ, neo, Fc, DIG, myc, and FLAG.

29. The prokaryotic vector of claim 27, wherein the vector is selected from the group consisting of pET, pET28, pcDNA3.1/V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT.

30. The eukaryotic vector of claim 27, wherein the vector is selected from the group consisting of MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pCEP4, and pEBVHis vectors.

31. A prokaryotic organism comprising the vector of claim 27.

32. An eukaryotic non human organism comprising the vector of claim 29.

33. A Bmpr1a mutant non human organism comprising a cell expressing an inactive Bmpr1a polypeptide.

34. The Bmpr1a mutant organism of claim 33, wherein the organism is selected from the group consisting of a transgenic Bmpr1a^{fx/fx} knockout organism and a conventional Bmpr1a mutant organism.

35. The Bmpr1a mutant organism of claim 33, comprising a vector, wherein the vector possesses a Bmpr1a mutation.

36. The Bmpr1a mutant organism of claim 35, wherein the Bmpr1a mutation is selected from the group consisting of frame shift, knockout deletion, conventional deletion, substitution, loss of function, and point mutations.

37. A vector comprising:
(a) a promoter; and,
(b) a gene selected from the group consisting of PTEN, Akt, GSK-3, cyclin D1, Tert polymerase, PI3K, SMAD 158, P27, and mutant genes derived therefrom.

38. The vector of claim 37, wherein mutation in the mutant gene is selected from the group consisting of frame shift, deletion, loss of function, substitution, and point mutations.

39. The vector of claim 37, wherein the vector is selected from the group consisting of expression vectors, fusion vectors, gene therapy vectors, two-hybrid vectors, reverse two-hybrid vectors, sequencing vectors, and cloning vectors.

40. The vector of claim 37, wherein the vector is selected from the group of eukaryotic vectors consisting of MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pCEP4, and pEBVHis vectors.

41. A hair follicle stem cell comprising the vector of claim 40.

42. A host non human organism comprising the hair follicle stem cell of claim 41.

43. A Bmpr1a mutant non human organism, wherein a hair follicle cell comprises a Bmpr1a receptor polypeptide selected from the group consisting of inactive and truncated Bmpr1a receptor polypeptides.

44. A mutant Bmpr1a non human organism comprising an inactivated Bmpr1a cell receptor polypeptide encoded by a mutant Bmpr1a gene selected from the group consisting of knockout deletion, conventional deletion, substitution, loss of function, and frame shift mutations.

45. The mutant Bmpr1a organism of claim 43, wherein the inactivated Bmpr1a receptor polypeptide is located in a hair follicle cell selected from the group consisting of stem, resting, self-renewing, proliferating, transient amplifying, differentiating, apoptotic, and tumor cells.

46. The mutant organizm of claim 45, wherein the tumor cell is a matricomas cell.

47. The mutant organism of claim 45, wherein the hair follicle cell is selected from the group consisting of a bulge cell and a hair shaft cell.

48. A knockout mutant Mx1-Cre⁺Bmpr1a^{fx/fx} non human organism, comprising a Bmpr1a receptor that has been substantially inactivated.

49. The knockout mutant Mx1-Cre⁺Bmpr1a^{fx/fx} organism of claim 48, comprising a matricomas.

50. A matricomas comprising a Bmpr1a receptor selected from the group consisting of an inactive Bmpr1a receptor and a truncated Bmpr1a receptor.

51. An in vitro hair follicle stem cell cultivation system, wherein a stem cell population proliferates, comprising:
(a) an isolated hair follicle stem cell population comprising at least 10⁴ cells;
(b) a culture medium; and,
(c) an effective amount of isolated Noggin polypeptides operatively bound in vitro to Bmpr1a cell receptors, wherein Bmpr1a receptor binding to BMP is substantially inhibited.

52. The in vitro hair follicle stem cell cultivation system of claim 51, wherein the isolated Noggin polypeptides are truncated polypeptides.

53. An in vitro hair follicle stem cell cultivation system, wherein a hair follicle stem cell population proliferates, comprising:
(a) an isolated hair follicle stem cell population comprising at least 10⁴ cells;
(b) a culture medium; and,
(c) antibodies selected from the group consisting of anti-Bmpr1a antibodies, anti-BMP antibodies, and fragments thereof.

54. An in vitro mutant Bmpr1a HFSC cultivation system, wherein a mutant HFSC population proliferates, comprising:
(a) an isolated mutant Bmpr1a HFSC population of at least 10⁴ cells comprising inactive Bmpr1a cell receptors encoded by nonfunctional mutant Bmpr1a genes, wherein mutation was induced in vitro; and,
(b) a culture medium.

55. The in vitro mutant HFSC cultivation system of claim 54, comprising feeder cells.

56. The in vitro mutant HFSC cultivation system of claim 54, wherein cells of the mutant HFSC population comprise Bmpr1a gene mutations selected from the group consisting of frame shift, substitution, loss of function, and deletion mutations.

57. An in vitro HFSC cultivation system, wherein an activated HFSC population proliferates, comprising:
(a) an isolated HSFC population comprising at least 10⁴ cells;
(b) a culture medium;
(c) an isolated stem cell activator, wherein the activator is inserted in at least one stem cell in the population and is selected from the group consisting of mutant Bmpr1a receptor polypeptides, mutant Bmpr1a receptor nucleic acid sequences, wild type Bmpr1a receptor antisense sequences, and fragments thereof; and,
(d) an activator insertion device.

58. The in vitro HFSC cultivation system of claim 57, wherein the activator insertion device is selected from the group consisting of injection, electroporation, transfection, vector, particle encapsulation, and liposome encapsulation devices.

59. The in vitro HFSC cultivation system of claim 57, comprising feeder cells.

60. An in vitro HFSC cultivation system comprising:
(a) an isolated HFSC population comprising at least 104 cells;
(b) Bmpr1a antisense oligonucleotides, wherein the Bmpr1a antisense oligonucleotides hybridize with Bmpr1a mRNA sequences in cells of the HFSC population to prevent Bmpr1a mRNA translation;
(c) an oligonucleotide insertion device, wherein the oligonucleotide insertion device is selected from the group consisting of injection, electroporation, transfection, vector, particle encapsulation, and liposome encapsulation devices; and,
(d) a culture medium.

61. A method for forming a pre-excision Mx1-Cre-Lox Bmpr1a^{fx/fx} knockout mutant organism for use in studying a hair follicle stem cell, comprising:
(a) isolating a Bmpr1a gene;
(b) forming a modified Bmpr1a gene, wherein the modified Bmpr1 gene comprises recombination sites;
(c) forming a Bmpr1a vector by insertion of the modified Bmpr1a gene into a vector;
(d) transfecting an embryonic stem cell with the Bmpr1a vector to form a Bmpr1a embryonic stem cell;
(e) inserting the Bmpr1a embryonic stem cell into a host uterus, wherein a Bmpr1a^{fx/fx} organism is formed; and,
(f) crossing the Bmpr1a^{fx/fx} organism with an Mx1-Cre organism to form a hybrid Mx1-Cre-Lox Bmpr1a^{fx/fx} organism.

62. The method of claim 61, wherein Bmpr1a vector formation comprises:
(a) inserting Lox recombination sites flanking Exon 2 of the Bmpr1a gene; and,
(b) inserting marker sites into the vector's genomic sequence.

63. A modified Bmpr1a nucleic acid sequence comprising a Bmpr1a nucleic acid sequence and two recombination sites, wherein the recombination sites flank a region of the Bmpr1a sequence.

64. The modified sequence of claim 63, wherein the recombination site is selected from the group consisting of a Lox site and an FRT site.

65. A post-excision mutant Bmpr1a sequence derived from the modified sequence of claim 63, wherein an activator induced recombination at the recombination sites to excise the flanked Bmpr1a sequence region.

66. The mutant sequence of claim 65, wherein the activator is selected from the group consisting of Cre recombinase and Flp recombinase.

67. The mutant sequence of claim 65, wherein the excised flanked Bmpr1a sequence region is Exon 2.

68. A method for forming a post-excision Mx1-Cre⁺Bmpr1a^{fx/fx} knockout mutant organism for use in studying a hair follicle cell, comprising:
(a) forming a hybrid pre-excision Mx1-Cre-Lox Bmpr1a^{fx/fx} mutant organism by the method of claim 61; and,
(b) administering a recombination activator to the hybrid pre-excision Mx1-Cre Bmpr1a^{fx/fx} organism, wherein Cre-mediated Lox site-directed Bmpr1a gene recombination is induced to yield an inactive Bmpr1a receptor.

69. The method of claim 68, comprising selecting the post-excision Bmpr1a^{fx/fx} knockout mutant organism.

70. The method of claim 68, wherein the hair follicle cell comprises an inactive Bmpr1a receptor polypeptide.

71. The method of claim 68, wherein the hair follicle cell is selected from the group consisting of HFSC, resting, self-renewing, proliferating, transient amplifying, differentiating, and apoptotic cells.

72. A method for obtaining a mutant phenotypic change in a hair follicle cell in vivo, wherein the phenotypic change is selected from the group consisting of increased HFSC population number, hair loss, and matricomas, comprising:
(a) isolating a Bmpr1a^{fx/fx} gene in a wild type Bmpr1a organism;
(b) forming a modified Bmpr1a gene, wherein the modified Bmpr1 gene comprises Lox recombination sites and marker sites;
(c) forming a Bmpr1a vector by insertion of the modified Bmpr1a gene into a vector;
(d) transfecting an embryonic stem cell with the Bmpr1a vector to form a Bmpr1a embryonic stem cell;
(e) inserting the Bmpr1a embryonic stem cell into a host uterus, wherein a Bmpr1a^{fx/fx} organism is formed;
(f) crossing the Bmpr1a^{fx/fx} organism with an Mx1-Cre organism to form a hybrid Mx1-Cre-Lox Bmpr1a^{fx/fx} organism; and,
(g) injecting a recombination activator into the hybrid Mx1-Cre-Lox Bmpr1a^{fx/fx} organism, wherein recombination results in expression of inactive Bmpr1a cell receptors.

73. A method for forming a post-excision Mx1-Cre⁺Bmpr1a^{fx/fx} Z/EG knockout mutant organism for use in studying a hair follicle cell comprising:
(a) making a pre-excision Mx1-Cre Lox Bmpr1a^{fx/fx} knockout mutant organism by the method of claim 61;
(b) crossing the Mx1-Cre Lox Bmpr1a^{fx/fx} organism with a Z/EG organism, wherein a pre-excision hybrid Mx1-Cre Lox Bmpr1a^{fx/fx} Z/EG organism is formed; and,
(c) administering a recombination activator to the hybrid Mx1-Cre Lox Bmpr1a^{fx/fx} Z/EG organism, wherein Cre-mediated Lox site-directed intracellular Bmpr1a gene recombination is induced.

74. A method for increasing a mutant HFSC population number in vitro comprising:
(a) isolating a Bmpr1a mutant HFSC population comprising at least 10⁴ cells; and,
(b) cultivating the HFSC population in vitro.

75. The method of claim 74, comprising placing the isolated mutant HFSC population in operative contact with feeder cells in vitro.

76. A method for increasing HFSC population number in vitro comprising:
(a) isolating a wild type HFSC population comprising at least 10⁴ cells;
(b) forming antibodies selected from the group consisting of anti-Bmpr1a, anti-BMP antibodies, and fragments thereof; and,
(c) placing the antibodies in operative contact with the HSFC population, wherein the antibodies inhibit BMP interaction with Bmpr1a cell receptors.

77. Isolated antibodies selected from the group consisting of anti-Bmpr1a, anti-BMP antibodies and fragments thereof, wherein the antibodies inhibit BMP interaction with Bmpr1a cell receptors.

78. A method for increasing HFSC population number in vitro comprising:
(a) isolating a wild type HFSC population comprising at least 10⁴ cells;
(b) forming Bmpr1a antisense oligonucleotide sequences;
(c) isolating the Bmpr1a antisense oligonucleotide sequences;
(d) administering the isolated Bmpr1a antisense oligonucleotide sequences into the HFSC population in vitro using an insertion device, wherein the oligonucleotide sequences operatively hybridize with Bmpr1a mRNA sequences to prevent intracellular translation of Bmpr1a polypeptides; and,
(e) cultivating the HFSC population in vitro.

79. The method of claim 78, wherein the insertion device for the administration of the isolated antisense oligonucleotides into the HFSC population is selected from the group consisting of injection, transfection, particle encapsulation, liposome-encapsulation, particle delivery, and electroporation devices.

80. The method of claim 78, wherein the forming of the isolated Bmpr1a antisense oligonucleotide sequence is a method selected from the group consisting of nucleic acid synthesis and nucleic acid cleavage.

81. The method of claim 78, comprising amplifying the isolated Bmpr1a antisense oligonucleotides.

82. An isolated Bmpr1a antisense oligonucleotide sequence, wherein the antisense oligonucleotide sequence operatively hybridizes with a Bmpr1a mRNA sequence to inhibit intracellular translation of a Bmpr1a polypeptide.

83. A kit for detecting marker polypeptides associated with hair follicle lineage commitment, the kit comprising:
(a) a container; and
(b) at least two labeled antibodies selected from the group of antibodies to BMP, Noggin, PTEN, P-PTEN, β-catenin, tert, PI3K, Akt, GSK3 β, and TCF3.

84. A kit for detecting mutant BMP pathway signaling in hair follicle tissue, the kit comprising,
(a) a container;
(b) a mutant Wt hair follicle tissue; and
(c) at least two labeled antibodies selected from the group of antibodies to BMP, Noggin, PTEN, P-PTEN, β-catenin, tert, PI3K, Akt, GSK3 β, and TCF3.

85. A kit for detecting mutant Wnt pathway signaling in hair follicle tissue, the kit comprising,
(a) a container;
(b) a mutant Wt hair follicle tissue; and
(c) at least two labeled antibodies selected from the group of antibodies to BMP, Noggin, PTEN, P-PTEN, β-catenin, tert, PI3K, Akt, GSK3 β, and TCF3.

86. The kit of claim 83, wherein the kit further comprises a control Wt hair follicle tissue.

87. The kit of claim 86, wherein the label is selected from the group consisting of fluorescent, phosphorescent, luminescent, radioactive, and chromogenic.

88. A kit for detecting mutant Bmpr1a nucleic acid sequences in hair follicle tissue, the kit comprising:
(a) a container;
(b) at least one nucleic acid sequence probe, wherein the probe hybridizes to a mutant Bmpr1a sequence region; and
(c) a hair follicle tissue selected from the group consisting of Bmpr1a mutant and Wt tissue.

89. A Western Blot kit for detecting a mutant Bmpr1a polypeptide sequence in hair follicle tissue, the kit comprising;
(a) a container;
(b) Bmpr1a polypeptide standards; and
(c) At least one primary antibody selected from the group consisting of antibodies to Wt Bmpr1a and mutant Bmpr1a polypeptides; and
(d) At least one secondary antibody that is labeled, wherein the binding of labeled secondary antibody to the primary antibody permits detection of the mutant Bmpr1a polypeptide in the hair follicle tissue.

90. A marker for identifying HFSC self-renewal, the marker comprising a mutant Bmpr1a polypeptide, wherein the polypeptide has a loss of receptor function.

91. A marker for identifying HFSC differentiation, the marker selected from the group consisting of Bmp1a, BMP, PTEN, P-PTEN, AKT, and P-AKT.

92. An isolated hair follicle stem cell population characterized as being Bmpr1a⁻.

93. An isolated hair follicle stem cell population wherein BMP4 is overexpressed.

94. A method to promote HFSC expansion, the method comprising preventing the binding of BMP to Bmpr1a.

95. A method to promote epidermal stem cell expansion, the method comprising overexpressing BMP.


SEQUENCE LISTING (17 sequences)

SEQ ID NO: 1 - DNA, 2056 bp, Mus musculus
cgaattcctcgcgccgtgggaggggcggcccggcccacccccacgccccgcccgggagggacggggggagagagagcgcggcgacgggtatctgggtcaaagctgttcggagaaattggaactacagttttatctagccacatctctgagaattctgaagaaagcagcaggtgaaagtcattgccaagtgattttgttctgtaaggaagcctccctcattcacttacaccagtgagacagcaggaccagtcattcaaagggccgtgtacaggacgcgtgcgaatcagacaatgactcagctatacacttacatcagattactgggagcctgtctgttcatcatttctcatgttcaagggcagaatctagatagtatgctccatggcactggtatgaaatcagacttggaccagaagaagccagaaaatggagtgactttagcaccagaggataccttgcctttcttaaagtgctattgctcaggacactgcccagatgatgctattaataacacatgcataactaatggccattgctttgccattatagaagaagatgatcagggagaaaccacattaacttctgggtgtatgaagtatgaaggctctgattttcaatgcaaggattcaccgaaagcccagctacgcaggacaatagaatgttgtcggaccaatttgtgcaaccagtatttgcagcctacactgccccctgttgttataggtccgttctttgatggcagcatccgatggctggttgtgctcatttccatggctgtctgtatagttgctatgatcatcttctccagctgcttttgctataagcattattgtaagagtatctcaagcaggggtcgttacaaccgtgatttggaacaggatgaagcatttattccagtaggagaatcattgaaagacctgattgaccagtcccaaagctctgggagtggatctggattgcctttattggttcagcgaactattgccaaacagattcagatggttcggcaggttggtaaaggccgctatggagaagtatggatgggtaaatggcgtggtgaaaaagtggctgtcaaagtgttttttaccactgaagaagctagctggtttagagaaacagaaatctaccagacggtgttaatgcgtcatgaaaatatacttggttttatagctgcagacattaaaggcactggttcctggactcagctgtatttgattactgattaccatgaaaatggatctctctatgacttcctgaaatgtgccacactagacaccagagccctactcaagttagcttattctgctgcttgtggtctgtgccacctccacacagaaatttatggtacccaagggaagcctgcaattgctcatcgagacctgaagagcaaaaacatccttattaagaaaaatggaagttgctgtattgctgacctgggcctagctgttaaattcaacagtgatacaaatgaagttgacatacccttgaataccagggtgggcaccaagcggtacatggctccagaagtgctggatgaaagcctgaataaaaaccatttccagccctacatcatggctgacatctatagctttggtttgatcatttgggaaatggctcgtcgttgtattacaggaggaatcgtggaggaatatcaattaccatattacaacatggtgcccagtgacccatcctatgaggacatgcgtgaggttgtgtgtgtgaaacgcttgcggccaatcgtgtctaaccgctggaacagcgatgaagtaagttggagccaagtccctgtaaagtgatgagtgagtgccgagttactctgtgctcaccacactctgtttgcatttatttctctttagtgtcttcgagcagttttgaagctaatgtcagaatgttgggcccataatccagcctccagactcacagctttgagaatcaagaagacacttgcaaaaatggttgaatcccaggatgtaaagatttgacaattaaacaattttgagggagaatttagactgcaagaacttcttcacccaaggaaggaattcctgcaggcccgggg

SEQ ID NO: 2 - DNA, 1891 bp, Mus musculus
cgaattcctcgcgccgtgggaggggcggcccggcccacccccacgccccgcccgggagggacggggggagagagagcgcggcgacgggtatctgggtcaaagctgttcggagaaattggaactacagttttatctagccacatctctgagaattctgaagaaagcagcaggtgaaagtcattgccaagtgattttgttctgtaaggaagcctccctcattcacttacaccagtgagacagcaggaccagtcattcaaagggccgtgtacaggacgcgtgcgaatcagacaatgactcagctatacacttacatcagattactgggagcctgtctgttcatcatttctcatgttcaaactaatggccattgctttgccattatagaagaagatgatcagggagaaaccacattaacttctgggtgtatgaagtatgaaggctctgattttcaatgcaaggattcaccgaaagcccagctacgcaggacaatagaatgttgtcggaccaatttgtgcaaccagtatttgcagcctacactgccccctgttgttataggtccgttctttgatggcagcatccgatggctggttgtgctcatttccatggctgtctgtatagttgctatgatcatcttctccagctgcttttgctataagcattattgtaagagtatctcaagcaggggtcgttacaaccgtgatttggaacaggatgaagcatttattccagtaggagaatcattgaaagacctgattgaccagtcccaaagctctgggagtggatctggattgcctttattggttcagcgaactattgccaaacagattcagatggttcggcaggttggtaaaggccgctatggagaagtatggatgggtaaatggcgtggtgaaaaagtggctgtcaaagtgttttttaccactgaagaagctagctggtttagagaaacagaaatctaccagacggtgttaatgcgtcatgaaaatatacttggttttatagctgcagacattaaaggcactggttcctggactcagctgtatttgattactgattaccatgaaaatggatctctctatgacttcctgaaatgtgccacactagacaccagagccctactcaagttagcttattctgctgcttgtggtctgtgccacctccacacagaaatttatggtacccaagggaagcctgcaattgctcatcgagacctgaagagcaaaaacatccttattaagaaaaatggaagttgctgtattgctgacctgggcctagctgttaaattcaacagtgatacaaatgaagttgacatacccttgaataccagggtgggcaccaagcggtacatggctccagaagtgctggatgaaagcctgaataaaaaccatttccagccctacatcatggctgacatctatagctttggtttgatcatttgggaaatggctcgtcgttgtattacaggaggaatcgtggaggaatatcaattaccatattacaacatggtgcccagtgacccatcctatgaggacatgcgtgaggttgtgtgtgtgaaacgcttgcggccaatcgtgtctaaccgctggaacagcgatgaagtaagttggagccaagtccctgtaaagtgatgagtgagtgccgagttactctgtgctcaccacactctgtttgcatttatttctctttagtgtcttcgagcagttttgaagctaatgtcagaatgttgggcccataatccagcctccagactcacagctttgagaatcaagaagacacttgcaaaaatggttgaatcccaggatgtaaagatttgacaattaaacaattttgagggagaatttagactgcaagaacttcttcacccaaggaaggaattcctgcaggcccgggg

SEQ ID NO: 3 - DNA, 165 bp, Mus Musulus
gggcagaatctagatagtatgctccatggcactggtatgaaatcagacttggaccagaagaagccagaaaatggagtgactttagcaccagaggataccttgcctttcttaaagtgctattgctcaggacactgcccagatgatgctattaataacacatgcata

SEQ ID NO: 4 - protein, 500 aa, Mus musculus
MTQLYTYIRLLGACLFIISHVQGQNLDSMLHGTGMKSDLDQKKPENGVTLAPEDTLPFLKCYCSGHCPDDAINNTCITNGHCFAIIEEDDQGETTLTSGCMKYEGSDFQCKDSPKAQLRRTIECCRTNLCNQYLQPTLPPVVIGPFFDGSIRWLVVLISMAVCIVAMIIFSSCFCYKHYCKSISSRGRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDIPLNTRVGTKRYMAPEVLDESLNKNHFQPYIMADIYSFGLIIWEMARRCITGGIVEEYQLPYYNMVPSDPSYEDMREVVCVKRLRPIVSNRWNSDEVSWSQVPVK

SEQ ID NO: 5 - protein, 456 aa, Mus musculus
MTQLYTYIRLLGACLFIISHVQGQNLDSMLHGTTNGHCFAIIEEDDQGETTLTSGCMKYEGSDFQCKDSPKAQLRRTIECCRTNLCNQYLQPTLPPVVIGPFFDGSIRWLVVLISMAVCIVAMIIFSSCFCYKHYCKSISSRGRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDIPLNTRVGTKRYMAPEVLDESLNKNHFQPYIMADIYSFGLIIWEMARRCITGGIVEEYQLPYYNMVPSDPSYEDMREVVCVKRLRPIVSNRWNSDEVSWSQVPVK

SEQ ID NO: 6 - protein, 44 aa, Mus musculus
GMKSDLDQKKPENGVTLAPEDTLPFLKCYCSGHCPDDAINNTCI

SEQ ID NO: 7 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 8 - DNA, 3631 bp, Homo sapiens
gcggccgctgcagagattggaatccgcctgccgggcttggcgaaggagaagggaggaggcaggagcgaggagggaggagggccaagggcgggcaggaaggcttaggctcggcgcgtccgtccgcgcgcggcgaagatcgcacggcccgatcgaggggcgaccgggtcggggccgctgcacgccaagggcgaaggccgattcgggccccacttcgccccggcggctcgccgcgcccacccgctccgcgccgagggctggaggatgcgttccctggggtccggacttatgaaaatatgcatcagtttaatactgtcttggaattcatgagatggaagcataggtcaaagctgtttggagaaaatcagaagtacagttttatctagccacatcttggaggagtcgtaagaaagcagtgggagttgaagtcattgtcaagtgcttgcgatcttttacaagaaaatctcactgaatgatagtcatttaaattggtgaagtagcaagaccaattattaaaggtgacagtacacaggaaacattacaattgaacaatgcctcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagacattcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcacccactcctttacaaccatactttatatatgtacatacattcatactgtagaaaccagctcatgtgtacctcatatcccatccttaagagaagaaatgttataaagtagaactaaatataaattttcagaattaatgcattcaaagtaatatatcaaatccaggactttgttaacttcaggtaaaaacttcattagggtaatatcatctcaattttttcaaatgaaaggattctctaattagaaatttatatgtcagagctgttataaatttatcaactgtcaaatatgttctggacagctaaatcatttgagatttttggttttttgatttctattccctaacttgtgaagacaatgaaaaatcaggcagaaatatttagtatctagtcagtatctgtagctacactgtataactgttcttcaataaaatggttcatattttatagaaaaaaaaaaaaaaaa

SEQ ID NO: 9 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 10 - DNA, 1279 bp, Homo sapiens
cagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaaaaaaaaaa

SEQ ID NO: 11 - protein, 232 aa, Mus musculus
MERCPSLGVTLYALVVVLGLRAAPAGGQHYLHIRPAPSDNLPLVDLIEHPDPIFDPKEKDLNETLLRSLLGGHYDPGFMATSPPEDRPGGGGGPAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 12 - DNA, 699 bp, Mus musculus
atggagcgctgccccagcctgggggtcaccctctacgccctggtggtggtcctggggctgcgggcagcaccagccggcggccagcactatctacacatccgcccagcacccagcgacaacctgcccttggtggacctcatcgaacatccagaccctatctttgaccctaaggagaaggatctgaacgagacgctgctgcgctcgctgctcgggggccactacgacccgggctttatggccacttcgcccccagaggaccgacccggagggggcgggggaccggctggaggtgccgaggacctggcggagctggaccagctgctgcggcagcggccgtcgggggccatgccgagcgagatcaaagggctggagttctccgagggcttggcccaaggcaagaaacagcgcctgagcaagaagctgaggaggaagttacagatgtggctgtggtcacagaccttctgcccggtgctgtacgcgtggaatgacctaggcagccgcttttggccacgctacgtgaaggtgggcagctgcttcagcaagcgctcctgctctgtgcccgagggcatggtgtgtaagccatccaagtctgtgcacctcacggtgctgcggtggcgctgtcagcggcgcgggggtcagcgctgcggctggattcccatccagtaccccatcatttccgagtgtaagtgttcctgctag

SEQ ID NO: 13 - DNA, 21 bp, Artificial
  Primer
gcagctgctgctgcagcctcc

SEQ ID NO: 14 - DNA, 21 bp, Artificial
  Primer
tggctacaatttgtctcatgc

SEQ ID NO: 15 - DNA, 21 bp, Artificial
  Primer
ggtttggatcttaaccttagg

SEQ ID NO: 16 - DNA, 20 bp, Artificial
  Primer
tacctggaaaatgcttctgt

SEQ ID NO: 17 - DNA, 20 bp, Artificial
  Primer
tgatctccggtattgaaact